(19) United States
(12) Patent Application Publication
(10) Pub. No.: US 2021/0206864 A1
(43) Pub. Date: Jul. 8, 2021

(54) ANTI-OX40 ANTAGONISTIC ANTIBODIES AND DOSAGE FOR THE TREATMENT OF OX40-MEDIATED DISORDERS

(71) Applicant: ICHNOS SCIENCES SA, La Chaux-de-Fonds (CH)

(72) Inventors: Jonathan BACK, La Chaux-de-Fonds (CH); Sachin DUBEY, La Chaux-de-Fonds (CH)

(21) Appl. No.: 17/058,728

(22) PCT Filed: May 29, 2019
(86) PCT No.: PCT/EP2019/064028

§ 371 Date: Nov. 25, 2020

(30) Foreign Application Priority Data
May 31, 2018 (EP) 18175396.3
Feb. 22, 2019 (EP) 19158921.7

(51) Int. Cl. C07K 16/28; A61P 37/08

(52) U.S. Cl. CPC C07K 16/2878; A61K 2039/505; A61P 37/08

(57) ABSTRACT

The present invention relates to an anti-OX40 antagonist antibody for use in the treatment or prevention of OX40-mediated disorders.

### TECHNICAL FIELD

The present invention relates to an anti-OX40 antagonist antibody for use in the treatment or prevention of OX40-mediated disorders.

### BACKGROUND

OX40 (CD134) is a member of the tumor necrosis factor (TNF) receptor gene family. OX40 is predominantly expressed on activated T cells including CD4 and CD8 T cells; T-helper type 1, type 2, and type 17 cells; and forkhead box P3 positive/CD4+ regulatory T cells. The interaction between OX40 and its ligand OX40L (CD252) plays an important role in antigen-specific T cell expansion and survival. OX40 is expressed predominantly on T cells early after antigen activation. In contrast, OX40L is expressed mainly on activated antigen presenting cells and endothelial cells during inflammation. Ligation of OX40 by OX40L leads to enhanced T cell survival and proliferation and drives beneficial inflammatory processes as well as pathological autoimmune diseases.

It has been suggested that OX40 antagonism would be useful in reducing harmful autoimmune responses mediated by activated T cells and especially by memory T cells. This can be achieved by targeting the OX40/OX40L interaction with blocking antibodies against either OX40L or OX40. In this regard, blocking the OX40/OX40L pathway was shown to be protective in several animal models of human disease such as asthma, inflammatory bowel disease, transplant rejection, autoimmune diabetes, graft versus host disease (GvHD), arthritis, and experimental autoimmune encephalomyelitis, validating OX40 as a highly attractive pathway to antagonize in autoimmune diseases (Croft, 2010). The tissue-specific nature of these autoimmune responses and their characteristic chronicity and relapse/remitting patterns suggest that activated memory T cells and not recently activated T cells are driving the disease process. In vivo blockade of OX40 ligand inhibits thymic stromal lymphopoietin driven atopic inflammation (Seshasayee et al, 2007; Webb et al, 2016).

GBR830 (CAS Registry Number 2126777-87-3) is a humanized, immunoglobulin G1 (IgG1) antibody specific for OX40. Blocking the binding of OX40 to its ligand OX40L consequently reduces the longevity and severity of the inappropriate immune responses and thus gives GBR830 the potential to treat T cell pathology related autoimmune diseases.

Treatments of patients suffering of an OX40-mediated disease or prevention of such disease requires the development of proper administration strategies and dosages.

### SUMMARY OF THE INVENTION

The present invention relates to an anti-OX40 antagonist antibody for use in the treatment or prevention of a OX40-mediated disorder.

More specifically, the present invention relates to an anti-OX40 antagonist antibody for use in the treatment of an OX40-mediated disorder, wherein said antibody is suitable for intravenous administration at least a single dose of at least 300 mg; or wherein said antibody is suitable for subcutaneous administration at least a single dose of 50 mg.

In one aspect, the present invention relates to an anti-OX40 antagonist antibody for use in the treatment of an OX40-mediated disorder that is suitable for intravenous administration
(a) at a single dose comprised between about 10 mg/kg of a subject body weight and about 50 mg/kg of a subject body weight; or
(b) at a single dose equal to or less than 3 g; or
(c) at multiple doses of about 1 mg/kg of a subject body weight to about 30 mg/kg of a subject body weight administrated for at least two consecutive weeks at least once a week.

In another aspect, the present invention relates to an anti-OX40 antagonist antibody for use in the treatment of an OX40-mediated disorder that is suitable for subcutaneous administration
(a) at a single dose comprised between about 50 mg and about 1 g; or
(b) at a loading dose comprised between about 50 mg and about 1.5 g on Day 1, followed by at least one maintenance dose comprised between about 20 mg and about 1 g, starting on a day comprised between Day 10 and Day 40.

In one aspect, the disclosed antibody is suitable for intravenous administration of a single dose of:
(a) about 20 mg/kg of a subject body weight and following administration, pharmacokinetics parameters of said antibody comprise Cmax comprised between about 400 mcg/mL and about 800 mcg/mL and t1/2 comprised between about 200 hours and about 500 hours; or
(b) about 40 mg/kg of a subject body weight and following administration, pharmacokinetics parameters of said antibody comprise Cₘₐₓ comprised between about 900 mcg/mL and about 1300 mcg/mL and t1/2 comprised between about 300 hours and about 600 hours; or
(c) about 600 mg, wherein following administration, pharmacokinetics parameters of said antibody comprise Cₘₐₓ comprised between about 100 μg/mL and about 300 μg/mL and t1/2 comprised between about 200 hours and about 500 hours.
(d)

In a further aspect, the disclosed antibody is suitable for intravenous administration at a multiple dose of:
(a) about 10 mg/kg of a subject body weight for at least 6 consecutive weeks at least once a week, and following administration, pharmacokinetics parameters of said antibody comprise Cₘₐₓ comprised between about 200 mcg/mL and about 400 mcg/mL at week 1, comprised between about 400 mcg/mL and about 700 mcg/mL at week 4, and comprised between about 500 mcg/mL and about 800 mcg/mL at week 6 and t1/2 comprised between about 200 hours and about 500 hours at week 6; or
(b) about 20 mg/kg of a subject body weight, for at least 6 consecutive weeks at least once a week, and following administration, pharmacokinetics parameters of said antibody comprise Cmax comprised between about 400 mcg/mL and about 700 mcg/mL at week 1, comprised between about 900 mcg/mL and about 1300 mcg/mL at week 4, and comprised between about 1000 mcg/mL and about 1400 mcg/mL at week 6 and t1/2 comprised between about 300 hours and about 600 hours at week 6.

In another aspect, the disclosed antibody is suitable for subcutaneous administration at a single dose of:
(a) about 600 mg, and following administration, pharmacokinetics parameters of said antibody comprise Cₘₐₓ comprised between about 30 μg/mL and about 90 μg/mL and t1/2 comprised between about 200 hours and about 500 hours; or
(b) about 75 mg, and following administration, pharmacokinetics parameters of said antibody comprise Cmax comprised between about 2 μg/mL and about 18 μg/mL and t1/2 comprised between about 100 hours and about 400 hours.

In another aspect, the disclosed antibody is suitable for subcutaneous administration
(a) at a loading dose comprised between about 300 mg and about 1 g on Day 1, followed by at least one maintenance dose comprised between about 100 mg and about 600 mg, starting on a day comprised between Day 10 and Day 20, or
(b) at a loading dose comprised between about 300 mg and about 1 g on Day 1, followed by at least one maintenance dose comprised between about 100 mg and about 600 mg, starting on a day comprised between Day 20 and Day 40, or
(c) at a loading dose comprised between about 50 mg and about 300 mg on Day 1, followed by at least one maintenance dose comprised between about 20 mg and about 150 mg, starting on a day comprised between Day 20 and Day 40.

In particular, the maintenance dose is administrated every n days thereafter, wherein n is comprised between 10 days and 20 days; or comprised between 20 days and 40 days.

In a more particular aspect, the antibody of the present invention is suitable for subcutaneous administration
(a) at loading dose of about 600 mg on Day 1, followed by a maintenance dose of about 300 mg starting at Day 15 every 2 weeks; or
(b) at loading dose of about 600 mg on Day 1, followed by a maintenance dose of about 300 mg starting at Day 29 every 4 weeks; or
(c) at loading dose of about 150 mg on Day 1, followed by a maintenance dose of about 75 mg starting at Day 29 every 4 weeks.

In a further aspect, the antibody of the present invention is suitable for subcutaneous administration to a subject wherein the subject has at least one characteristic selected from the group comprising:
(a) having AD involvement of 3.0% body surface area (BSA) at screening and baseline.
(b) having EASI score equal to or greater than about 12 at screening or equal to or greater than about 16 at baseline;
(c) having IGA score equal to or greater than about 3 at screening and at baseline;
(d) having Baseline Pruritus Numerical Rating Scale (NRS) score for maximum itch intensity equal to or greater than about 3 over the previous 24 hours.

According to the present invention, the OX40-mediate disorder is selected from the group comprising infections (viral, bacterial, fungal and parasitic, endotoxic shock associated with infection, arthritis, rheumatoid arthritis, asthma, bronchitis, influenza, respiratory syncytial virus, pneumonia, COPD, idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coehac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, wherein atopic dermatitis is mild, or mild-to-moderate, or moderate, or moderate-to-severe, or severe, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

In particular, OX40-mediated disorder selected from the group comprising atopic dermatitis wherein atopic dermatitis is mild, or mild-to-moderate, or moderate, or moderate-to-severe, or severe, rheumatoid arthritis, autoimmune uveitis, multiple sclerosis, lupus (such as systemic lupus erythematosus), ulcerative colitis, scleroderma and graft-versus-host disease (GVHD) and hidradenitis.

In a more specific aspect, the OX40-mediate disorder moderate-to-severe atopic dermatitis.

I certain embodiment, the antibody of the present invention is identified by the CAS Registry Number: 2126777-87-3.

The present invention also relates to a stable pharmaceutical formulation comprising the disclosed antibody.

The term “human OX40” as used herein includes variants, isoforms, and species homologs of human OX40. Accordingly, antibodies of this disclosure may, in certain cases, cross-react with OX40 from species other than human. In certain embodiments, the antibodies may be completely specific for one or more human OX40 proteins and may not exhibit species or other types of non-human cross-reactivity. The complete amino acid sequence of an exemplary human OX40 has Swiss-Prot accession number P43489. OX40 is also known as CD134, TNFRSF4, ACT35 or TXGP1 L. Human OX40 is designated GeneID: 7293 by Entrez Gene, and HGNC: 1 1918 by HGNC. OX40 has also been designated CD 134 (cluster of differentiation 134). OX40 can be encoded by the gene designated TNFRSF4/OX40. The use of “human OX40” herein encompasses all known or as yet undiscovered alleles and polymorphic forms of human OX40. The terms “human OX40”, “OX40” or “OX40 Receptor” are used herein equivalently and mean “human OX40” if not otherwise specifically indicated.

The use of “human OX40” herein encompasses all known or as yet undiscovered alleles and polymorphic forms of human OX40. The terms “human OX40”, “OX40” or “OX40 Receptor” are used herein equivalently and mean “human OX40” if not otherwise specifically indicated.

The term “OX40 ligand” or “OX40L” are used herein equivalently and include OX40 ligand, specifically human OX40 ligand. OX40L is a member of the TNF superfamily and is also known as gp34 or CD252. OX40L has also been designated CD252 (cluster of differentiation 252) and has the sequence database accession number P23510 (Swiss-Prot) or Q6FGS4 (Uniprot). OX40L is expressed on the surface of activated B cells, T cells, dendritic cells and endothelial cells.

The term “antibody or fragment thereof that binds to human OX40” as used herein includes antibodies or a fragment thereof that binds to human OX40 e.g. human OX40 in isolated form, with an affinity (KD) of 500 nM or less, preferably 200 nM or less, more preferably 150 nM or less, more preferably 120 nM or less, even more preferably 110 nM or less. The term “antibody or fragment thereof that binds to human OX40” includes antibodies or antigenic binding fragments thereof.

The terms “antagonistic antibody” or “antagonist antibody” are used herein equivalently and include an antibody that is capable of inhibiting and/or neutralising the biological signaling activity of OX40, for example by blocking binding or substantially reducing binding of OX40 to OX40 ligand and thus inhibiting or reducing the signalisation pathway triggered by OX40 and/or inhibiting or reducing an OX40-mediated cell response like lymphocyte proliferation, cytokine expression, or lymphocyte survival. The term “antibody” as referred to herein includes whole antibodies and any antigen binding fragments or single chains thereof.

An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The term “heavy chain variable framework region” as referred herein may comprise one or more (e.g., one, two, three and/or four) heavy chain framework region sequences (e.g., framework 1 (FW1), framework 2 (FW2), framework 3 (FW3) and/or framework 4 (FW4)). Preferably the heavy chain variable region framework comprises FW1, FW2 and/or FW3, more preferably FW1, FW2 and FW3. The term “light chain variable framework region” as referred herein may comprise one or more (e.g., one, two, three and/or four) light chain framework region sequences (e.g., framework 1 (FW1), framework 2 (FW2), framework 3 (FW3) and/or framework 4 (FW4)). Preferably the light chain variable region framework comprises FW1, FW2 and/or FW3, more preferably FW1, FW2 and FW3.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the First component (C1q) of the classical complement system.

Antibodies are grouped into classes, also referred to as isotypes, as determined genetically by the constant region. Human constant light chains are classified as kappa (CK) and lambda (CX) light chains. Heavy chains are classified as mu (μ), delta (δ), gamma (γ), alpha (a), or epsilon (ε), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgG1 (IGHG1), IgG2 (IGHG2), IgG3 (IGHG3), IgG4 (IGHG4), IgA1 (IGHA1), IgA2 (IGHA2), IgM (IGHM), IgD (IGHD), and IgE (IGHE). The so-called human immunoglobulin pseudo-gamma IGHGP gene represents an additional human immunoglobulin heavy constant region gene which has been sequenced but does not encode a protein due to an altered switch region (Bensmana M et al., (1988) Nucleic Acids Res. 16(7): 3108). In spite of having an altered switch region, the human immunoglobulin pseudo-gamma IGHGP gene has open reading frames for all heavy constant domains (CH1-CH3) and hinge. All open reading frames for its heavy constant domains encode protein domains which align well with all human immunoglobulin constant domains with the predicted structural features. This additional pseudo-gamma isotype is referred herein as IgGP or IGHGP. Other pseudo immunoglobulin genes have been reported such as the human immunoglobulin heavy constant domain epsilon PI and P2 pseudo-genes (IGHEP1 and IGHEP2). The IgG class is the most commonly used for therapeutic purposes. In humans this class comprises subclasses IgG1, IgG2, IgG3 and IgG4. In mice this class comprises subclasses IgG1, IgG2a, IgG2b, IgG2c and IgG3.

Provided by the present invention is an anti-OX40 antagonist antibody for use in the treatment of an OX40-mediated disorder. Also provided by the present invention is a method for treating an OX40 mediated disorder.

The present invention relates to an anti-OX40 antagonist antibody for use in the treatment of subjects suffering of an OX40-mediated disorders. Also provided by the present disclosure is a method for treating an OX40 mediated disorder by administering to a subject a therapeutically effective amount of the disclosed anti-OX40 antagonist antibody.

The present invention relates to an anti-OX40 antagonist antibody for use in the treatment of patients suffering of an OX40-mediated disorders. Also provided by the present disclosure is a method for treating an OX40 mediated disorder by administering to a patient a therapeutically effective amount of the disclosed anti-OX40 antagonist antibody.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Preferably the subject is human.

A “patient” for the purposes of the present invention includes both humans and other animals, preferably mammals and most preferably humans. Thus the antibodies of the present invention have both human therapy and veterinary applications. The term “treatment” or “treating” in the present invention is meant to include therapeutic treatment, as well as prophylactic, or suppressive measures for a disease or disorder. Thus, for example, successful administration of an antibody prior to onset of the disease results in treatment of the disease. As another example, successful administration of an antibody after clinical manifestation of the disease to combat the symptoms of the disease comprises treatment of the disease.

“Treatment” and “treating” also encompasses administration of an antibody after the appearance of the disease in order to eradicate the disease. Successful administration of an antibody after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

The antibody or of the present invention can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, for example, intranasally, orally, vaginally, rectally, sublingually or topically. In a preferred aspect of the present invention the anti-OX40 antagonist antibody is administered intravenously.

The antibody of the present invention can be administered at a single or multiple doses. The term “dose” as used in the present invention, indicates an amount of drug substance administered per body weight of a subject or a total dose administered to a subject irrespective to their body weight.

To study the time course of drug absorption, distribution, metabolism, and excretion to enhance the efficacy of the drug and decrease its toxicity, collectively known as pharmacokinetics (PK) studies, PK parameters can be investigated. Non limiting examples of PK parameters include: maximum observed serum concentration (Cmax), average plasma drug concentration (Cavg), trough plasma concentration (Ctrough), last measurable plasma concentration (Clast), area under the plasma concentration-time curve at time t (AUCt), e.g. AUC168 being the area under the concentration-time curve (time 0 to time 168 hours) area under the serum concentration time curve from time 0 to time of the last measurable concentration (AUC0-last), area under the plasma concentration-time curve from time zero to infinity (AUC0-inf) time of maximum observed serum concentration (Tmax), time of last observed serum concentration (Tlast), apparent terminal elimination half-life (t½), total clearance (CL), apparent volume of distribution associated with the terminal phase (Vz), volume of distribution at steady state (Vss), accumulation ratio (Rac). In particular, provided by the present invention is an anti-OX40 antagonist antibody for use in the treatment of an OX40-mediated disorder, wherein said anti-OX40 antibody is administered to a patient in need thereof intravenously or subcutaneously. Also provided by the present invention is a method for treating an OX40 mediated disorder, wherein said anti-OX40 antibody is administered to a patient in need thereof intravenously or subcutaneously.

PK parameter can be derived from the drug concentration in the serum. Serum concentrations of a drug, such as the antibody of the present invention, can be quantified using an enzyme-linked immunosorbent assay (ELISA) method. The ELISA method to quantify the concentration of the antibody of the present application comprises the following steps: (a) coting a plate surface with a capture protein, such as hOX40-His; (b) incubating the resulting coated plate with the antibody of the present invention; (c) applying a washing step to remove unbound antibody; (d) add a detecting antibody, such as goat anti-human IgG Fcγ fragment conjugated with horseradish peroxidase; (e) adding tetramethylbenzidine; (f) detect absorbance at 450 nm; (g) generation of a standard curve, i.e. by using a four parameter regression model with 1/γ2; wherein the calibration curve range is between about 6250 ng/ml to about 97.7 ng/ml in undiluted serum or between 25000 ng/ml to 390 ng/ml in undiluted serum.

In particular the present invention relates to an anti-OX40 antagonist antibody for use in the treatment of an OX40-mediated disorder, wherein said antibody is suitable for intravenous administration at least a single dose of at least 300 mg; or wherein said antibody is suitable for subcutaneous administration at least a single dose of 50 mg.

More in particular the present invention relates to an anti-OX40 antagonist antibody for use in the treatment of OX40-mediated disorders, wherein said anti-OX40 antibody is suitable for intravenous administration at a single dose comprised between about 10 mg/kg of a subject body weight and about 50 mg/kg of a subject body weight, or at a single dose equal to or less than 3 g; or at multiple doses of about 1 mg/kg of a subject body weight to about 30 mg/kg of a subject body weight administrated for at least two consecutive weeks at least once a week; or wherein said antibody is suitable for subcutaneous administration at a single dose comprised between about 50 mg and about 1 g; or at loading dose comprised between about 50 mg and about 1.5 g on Day 1, followed by at least one maintenance dose comprised between about 20 mg and about 1 g, starting on a day comprised between Day 10 and Day 40. The present disclosure also relates to a method for treating an OX40 mediated disorder by administering to a patient in need thereof the anti-OX40 antibody of the present invention intravenously at a single dose comprised between about 10 mg/kg of a subject body weight and about 50 mg/kg of a subject body weight, or at a single dose equal to or less than 3 g; or at multiple doses of about 1 mg/kg of a subject body weight of a subject body weight to about 30 mg/kg of a subject body weight of a subject body weight administrated for at least two consecutive weeks at least once a week; or subcutaneously at a single dose comprised between about 50 mg and about 1 g; or at loading dose comprised between about 50 mg and about 1.5 g on Day 1, followed by at least one maintenance dose comprised between about 20 mg and about 1 g, starting on a day comprised between Day 10 and Day 40.

In one embodiment, the antibody of the present invention is administrated intravenously at a dose comprised between about 1 mg/kg of a subject body weight and about 100 mg/kg of a subject body weight, e.g. comprised between about 3 mg/kg of a subject body weight of a subject body weight of a subject body weight and about 80 mg/kg of a subject body weight, between about 5 mg/kg of a subject body weight and about 50 mg/kg of a subject body weight, between about 10 mg/kg of a subject body weight and about 40 mg/kg of a subject body weight. In a particular embodiment the antibody of the present invention is administrated at a dose of at least about 1 mg/kg of a subject body weight, at least about 5 mg/kg of a subject body weight, at least about 10 mg/kg of a subject body weight, or at least about 15 mg/kg of a subject body weight, at least about 20 mg/kg of a subject body weight, at least about 25 mg/kg of a subject body weight, at least about 30 mg/kg of a subject body weight, at least about 35 mg/kg of a subject body weight, at least about 40 mg/kg of a subject body weight, at least about 45 mg/kg of a subject body weight, at least about 50 mg/kg of a subject body weight. In a more particular embodiment, the antibody of the present invention is administrated at a dose selected from the group comprising about 1 mg/kg of a subject body weight, about 5 mg/kg of a subject body weight, about 10 mg/kg of a subject body weight, about 15 mg/kg of a subject body weight, about 20 mg/kg of a subject body weight, about 25 mg/kg of a subject body weight, about 30 mg/kg of a subject body weight, about 35 mg/kg of a subject body weight, about 40 mg/kg of a subject body weight, about 45 mg/kg of a subject body weight, about 50 mg/kg of a subject body weight, about 60 mg/kg of a subject body weight, about 70 mg/kg of a subject body weight, about 80 mg/kg of a subject body weight, about 90 mg/kg of a subject body weight, about 100 mg/kg of a subject body weight. The present invention also includes administration doses at any intermediate value of the above said values.

In one embodiment, the antibody of the present invention is administrated intravenously at a dose equal to or less than 4 g, e.g. equal to or less than 3 g, equal to or less than 2 g, equal to or less than 1 g. The present invention also includes administration doses at any intermediate value of the above said values.

In one embodiment, the antibody of the present invention is administrated intravenously at a single dose. In another embodiment, the antibody of the present invention is administrated intravenously at multiple doses. In particular, the antibody administrated at multiple doses is administrated once a week for at least two weeks; preferably for at least 4 weeks, more preferably for 6 weeks.

In certain embodiment, when the antibody of the present invention is administered intravenously at a single dose of about 20 mg/kg of a subject body weight, pharmacokinetics parameters may have the following values:

Cmax is comprised between about 300 mcg/mL and about 900 mcg/mL, e.g. comprised between about 400 mcg/mL and about 800 mcg/mL, comprised between about 449 mcg/mL and about 753 mcg/mL. In particular Cmax is selected from the group comprising about 300 mcg/mL, about 350 mcg/mL, about 400 mcg/mL, about 450 mcg/mL, about 500 mcg/mL, about 550 mcg/mL, about 570 mcg/mL, about 600 mcg/mL, about 650 mcg/mL, about 700 mcg/mL, about 750 mcg/mL, about 800 mcg/mL, about 850 mcg/mL, about 900 mcg/mL. The present invention also includes Cmax at any intermediate value of the above said values;

Tmax is comprised between about 30 min and about 5 hours, e.g. comprised between about 1 hour and about 4. In particular tmax is selected from the group comprising about 30 min, about 1 hour, about 1.5 hour, about 2 hours, about 3 hour, about 4 hours, about 5 hours. The present invention also includes tmax at any intermediate value of the above said values;

AUC 0-168 is comprised between about 30000 mcg·h/mL and about 70000 mcg·h/mL, e.g. comprised between about 40000 mcg·h/mL and about 60000 mcg·h/mL, comprised between about 44000 mcg·h/mL and about 57000 mcg·h/mL, comprised between about 44350 mcg·h/mL and about 56530 mcg·h/mL. In particular AUC 0-168 is selected from the group comprising about 30000 mcg·h/mL, about 35000 mcg·h/mL, about 40000 mcg·h/mL, about 45000 mcg·h/mL, about 50000 mcg·h/mL, about 51000 mcg·h/mL, about 55000 mcg·h/mL, about 60000 mcg·h/mL, about 65000 mcg·h/mL, about 70000 mcg·h/mL. The present invention also includes AUC 0-168 at any intermediate value of the above said values;

AUC 0-last is comprised between about 120000 mcg·h/mL and about 190000 mcg·h/mL, e.g. comprised between about 130000 mcg·h/mL and about 180000 mcg·h/mL, comprised between about 130400 mcg·h/mL and about 178800 mcg·h/mL. In particular AUC 0-last is selected from the group comprising about 120000 mcg·h/mL, about 130000 mcg·h/mL, about 135000 mcg·h/mL, about 140000 mcg·h/mL, about 145000 mcg·h/mL, about 150000 mcg·h/mL, about 155000 mcg·h/mL, about 160000 mcg·h/mL, about 165000 mcg·h/mL, about 170000 mcg·h/mL, about 175000 mcg·h/mL, about 180000 mcg·h/mL. The present invention also includes AUC 0-last at any intermediate value of the above said values;

AUC 0-infinity is comprised between about 120000 mcg·h/mL and about 200000 mcg·h/mL, e.g. comprised between about 130000 mcg·h/mL and about 195000 mcg·h/mL, comprised between about 134100 mcg·h/mL and about 190800 mcg·h/mL. In particular AUC 0-infinity is selected from the group comprising about 120000 mcg·h/mL, about 130000 mcg·h/mL, about 35000 mcg·h/mL, about 140000 mcg·h/mL, about 145000 mcg·h/mL, about 150000 mcg·h/mL, about 155000 mcg·h/mL, about 160000 mcg·h/mL, about 165000 mcg·h/mL, about 170000 mcg·h/mL, about 175000 mcg·h/mL, about 180000 mcg·h/mL, about 185000 mcg·h/mL, about 190000 mcg·h/mL, about 195000 mcg·h/mL. The present invention also includes AUC 0-infinity at any intermediate value of the above said values;

T1/2 is comprised between about 200 hours and about 500 hours, e.g. comprised between about 250 hour and about 500 hours, comprised between about 297 hour and about 462 hours. In particular t1/2 is selected from the group comprising about 200 hour, about 250 hour, about 300 hours, about 350 hours, about 370 hours, about 400 hours, about 450 hours, about 500 hours. The present invention also includes t1/2 at any intermediate value of the above said values;

CL is comprised between about 5 mL/h and about 15 mL/h, e.g. comprised between about 7 mL/h and about 14 mL/h, comprised between about 8.34 mL/h and about 13.7 mL/h. In particular CL is selected from the group comprising about 5 mL/h, about 8 mL/h, about 10 mL/h, about 12 mL/h, about 15 mL/h. The present invention also includes CL at any intermediate value of the above said values;

Vz is comprised between about 2 L and about 10 L, e.g. comprised between about 3 hour and about 9 L, comprised between about 4 L and about 7 L, comprised between about 4.17 L and about 6.58 L. In particular Vz is selected from the group comprising about 2 L, about 3 L, about 4 L, about 5 L, about 6 L, about 7 L, about 8 L, about 9 L, about 10 L. The present invention also includes Vz at any intermediate value of the above said values;

Vss is comprised between about 2 L and about 10 L, e.g. comprised between about 3 hour and about 9 L, comprised between about 3 hour and about 7 L, comprised between about 3.34 L and about 5 L. In particular Vss is selected from the group comprising about 2 L, about 3 L, about 4 L, about 5 L, about 6 L, about 7 L, about 8 L, about 9 L, about 10 L. The present invention also includes Vss at any intermediate value of the above said values;

In another embodiment, when the antibody of the present invention is administered at a single dose of about 40 mg/kg of a subject body weight, pharmacokinetics parameters may have the following values:

Cmax is comprised between about 800 mcg/mL and about 1400 mcg/mL, e.g. comprised between about 900 mcg/mL and about 1300 mcg/mL, comprised between about 977 mcg/mL and about 1290 mcg/mL. In particular Cmax is selected from the group comprising about 800 mcg/mL, about 850 mcg/mL, about 900 mcg/mL, about 950 mcg/mL, about 1000 mcg/mL, about 1100 mcg/mL, about 1200 mcg/mL, about 1300 mcg/mL, about 1400 mcg/mL. The present invention also includes Cmax at any intermediate value of the above said values;

Tmax is comprised between about 30 min and about 3 hours, e.g. comprised between about 1 hour and about 2. In particular tmax is selected from the group comprising about 30 min, about 1 hour, about 1.5 hour, about 2 hours, about 3 hour. The present invention also includes tmax at any intermediate value of the above said values;

AUC 0-168 is comprised between about 80000 mcg·h/mL and about 130000 mcg·h/mL, e.g. comprised between about 90000 mcg·h/mL and about 125000 mcg·h/mL, comprised between about 90980 mcg·h/mL and about 120100 mcg·h/mL. In particular AUC 0-168 is selected from the group comprising about 80000 mcg·h/mL, about 85000 mcg·h/mL, about 90000 mcg·h/mL, about 95000 mcg·h/mL, about 100000 mcg·h/mL, about 105000 mcg·h/mL, about 110000 mcg·h/mL, about 115000 mcg·h/mL, about 120000 mcg·h/mL, about 125000 mcg·h/mL, about 130000 mcg·h/mL. The present invention also includes AUC 0-168 at any intermediate value of the above said values;

AUC 0-last is comprised between about 150000 mcg·h/mL and about 500000 mcg·h/mL, e.g. comprised between about 200000 mcg·h/mL and about 450000 mcg·h/mL, comprised between about 247300 mcg·h/mL and about 410200 mcg·h/mL. In particular AUC 0-last is selected from the group comprising about 150000 mcg·h/mL, about 200000 mcg·h/mL, about 250000 mcg·h/mL, about 300000 mcg·h/mL, about 350000 mcg·h/mL, about 400000 mcg·h/mL, about 450000 mcg·h/mL, about 500000 mcg·h/mL. The present invention also includes AUC 0-last at any intermediate value of the above said values;

AUC 0-infinity is comprised between about 150000 mcg·h/mL and about 550000 mcg·h/mL, e.g. comprised between about 200000 mcg·h/mL and about 500000 mcg·h/mL, comprised between about 257800 mcg·h/mL and about 456300 mcg·h/mL. In particular AUC 0-infinity is selected from the group comprising about 150000 mcg·h/mL, about 200000 mcg·h/mL, about 250000 mcg·h/mL, about 300000 mcg·h/mL, about 350000 mcg·h/mL, about 400000 mcg·h/mL, about 450000 mcg·h/mL, about 500000 mcg·h/mL, about 550000 mcg·h/mL. The present invention also includes AUC 0-infinity at any intermediate value of the above said values;

T1/2 is comprised between about 200 hours and about 700 hours, e.g. comprised between about 300 hours and about 600 hours, comprised between about 350 hour and about 527 hours. In particular t1/2 is selected from the group comprising about 200 hour, about 250 hour, about 300 hours, about 350 hours, about 370 hours, about 400 hours, about 450 hours, about 500 hours, about 550 hours, about 600 hours, about 650 hours, about 700 hours. The present invention also includes t1/2 at any intermediate value of the above said values;

CL is comprised between about 5 mL/h and about 15 mL/h, e.g. comprised between about 7 mL/h and about 14 mL/h, comprised between about 6 mL/h and about 12 mL/h. In particular CL is selected from the group comprising about 5 mL/h, about 8 mL/h, about 10 mL/h, about 12 mL/h, about 15 mL/h.

The present invention also includes CL at any intermediate value of the above said values; Vz is comprised between about 2 L and about 10 L, e.g. comprised between about 3 L and about 9 L, comprised between about 4 L and about 7 L, comprised between about 4.17 L and about 6.58 L. In particular Vz is selected from the group comprising about 2 L, about 3 L, about 4 L, about 5 L, about 6 L, about 7 L, about 8 L, about 9 L, about 10 L. The present invention also includes Vz at any intermediate value of the above said values;

Vss is comprised between about 2 L and about 10 L, e.g. comprised between about 3 L and about 9 L, comprised between about 3 L and about 7 L, comprised between about 3.34 L and about 5 L. In particular Vss is selected from the group comprising about 2 L, about 3 L, about 4 L, about 5 L, about 6 L, about 7 L, about 8 L, about 9 L, about 10 L. The present invention also includes Vss at any intermediate value of the above said values;

In another embodiment, when the antibody of the present invention is administered at a single dose of about 600 mg, pharmacokinetics parameters may have the following values:

Cmax is comprised between about 50 μg/mL and about 400 μg/mL, e.g. comprised between about 100 μg/mL and about 300 μg/mL, between about 120 μg/mL and about 250 μg/mL, comprised between about 145 μg/mL and about 238 μg/mL. In particular Cₘₐₓ is selected from the group comprising about 50 μg/mL, about 100 μg/mL, about 150 μg/mL, about 190 μg/mL, about 200 μg/mL, about 250 μg/mL, about 300 μg/mL, about 350 μg/mL, about 400 μg/mL. The present invention also includes Cmax at any intermediate value of the above said values;

Tmax is comprised between about 0.5 hours and about 10 hours, e.g. comprised between about 0.5 hours and about 8 hours, comprised between about 1 hour and about 6 hours. In particular Tmax is selected from the group comprising about 0.5 hours, about 1 hour, about 2 hours, about 5 hours, about 6 hours, about 8 hours, about 10 hours. The present invention also includes Tmax at any intermediate value of the above said values;

AUC 0-168 is comprised between about 13000 μg*h/mL and about 22000 μg*h/mL, e.g. comprised between about 14000 μg*h/mL and about 21500 μg*h/mL, comprised between about 14600 μg*h/mL and about 21000 μg*h/mL. In particular AUC 0-168 is selected from the group comprising about 13000 μg*h/mL, about 14000 μg*h/mL, about 15000 μg*h/mL, about 17000 μg*h/mL, about 17000 μg*h/mL, about 18000 μg*h/mL, about 19000 μg*h/mL, about 20000 μg*h/mL, about 21000 μg*h/mL, about 22000 μg*h/mL. The present invention also includes AUC 0-168 at any intermediate value of the above said values;

AUC 0-last is comprised between about 45000 μg*h/mL and about 75000 μg*h/mL, e.g. comprised between about 46000 μg*h/mL and about 73000 μg*h/mL, comprised between about 46100 μg*h/mL and about 72400 μg*h/mL. In particular AUC 0-last is selected from the group comprising about 45000 μg*h/mL, about 50000 μg*h/mL, about 55000 μg*h/mL, about 60000 μg*h/mL, about 65000 μg*h/mL, about 70000 μg*h/mL, about 75000 μg*h/mL. The present invention also includes AUC 0-last at any intermediate value of the above said values;

AUC 0-infinity is comprised between about 45000 μg*h/mL and about 80000 μg*h/mL, e.g. comprised between about 47000 μg*h/mL and about 78000 μg*h/mL, comprised between about 48000 μg*h/mL and about 77000 μg*h/mL, comprised between about 48200 μg*h/mL and about 77400 μg*h/mL. In particular AUC 0-infinity is selected from the group comprising about 45000 μg*h/mL, about 50000 μg*h/mL, about 55000 μg*h/mL, about 60000 μg*h/mL, about 65000 μg*h/mL, about 70000 μg*h/mL, about 75000 μg*h/mL, about 80000 μg*h/mL. The present invention also includes AUC 0-infinity at any intermediate value of the above said values;

CL is comprised between about 5 mL/h and about 15 mL/h, e.g. comprised between about 6 mL/h and about 14 mL/h, comprised between about 7 mL/h and about 13 mL/h, comprised between about 7.5 mL/h and about 12.5 mL/h. In particular CL is selected from the group comprising about 5 mL/h, about 7 mL/h, about 10 mL/h, about 12 mL/h, about 15 mL/h. The present invention also includes CL at any intermediate value of the above said values;

Vz is comprised between about 2 L and about 8 L, e.g. comprised between about 3 L and about 7.5 L, comprised between about 4 L and about 7 L. In particular Vz is selected from the group comprising about 2 L, 4 L, 5 L, 6 L, 8L. The present invention also includes Vz at any intermediate value of the above said values;

Vss is comprised between about 2 L and about 8 L, e.g. comprised between about 3 L and about 7.5 L, comprised between about 4 L and about 7 L. In particular Vss is selected from the group comprising about 2 L, 4 L, 5 L, 6 L, 8L. The present invention also includes Vss at any intermediate value of the above said values;

t1/2 is comprised between about 150 hours and about 500 hours, e.g. comprised between about 200 hours and about 450 hours, comprised between about 280 hour and about 430 hours. In particular t1/2 is selected from the group comprising about 150 hours, about 200 hour, about 250 hours, about 300 hours, 350 about, 400 hours, about 450 hours, about 500 hours. The present invention also includes t1/2 at any intermediate value of the above said values;

In certain embodiment, when the antibody of the present invention is administered intravenously at a multiple dose of about 10 mg/kg of a subject body weight, for at least 6 consecutive weeks at least once a week, pharmacokinetics parameters may have the following values:

Cmax is comprised between about 200 mcg/mL and about 800 mcg/mL. In particular Cmax is comprised between about 200 mcg/mL and about 400 mcg/mL at week 1, e.g. comprised between about 249 mcg/mL and about 314 mcg/mL at week 1; Cmax is comprised between about 400 mcg/mL and about 700 mcg/mL at week 4, e.g. comprised between about 482 mcg/mL and about 623 mcg/mL at week 4; Cmax is comprised between about 500 mcg/mL and about 800 mcg/mL at week 6, e.g. comprised between about 601 mcg/mL and about 682 mcg/mL at week 6. More in particular, Cmax is selected from the group comprising about 200 mcg/mL, about 250 mcg/mL, about 300 mcg/mL, about 350 mcg/mL, about 400 mcg/mL, about 450 mcg/mL, about 500 mcg/mL, about 550 mcg/mL, about 600 mcg/mL, about 650 mcg/mL, about 700 mcg/mL, about 750 mcg/mL, about 800 mcg/mL. The present invention also includes Cmax at any intermediate value of the above said values;

Tmax is comprised between about 30 min and about 5 hours. In particular, tmax is comprised between about 1 hour and about 4.5 hours, at week 1, 4 and 6. In particular tmax is selected from the group comprising about 30 min, about 1 hour, about 1.5 hour, about 2 hours, about 3 hours, about 4 hours, about 5 hours. The present invention also includes tmax at any intermediate value of the above said values.

AUC 0-168 is comprised between about 20000 mcg·h/mL and about 90000 mcg·h/mL. In particular AUC 0-168 is comprised between about 20000 mcg·h/mL and about 30000 mcg·h/mL at week 1, e.g. AUC 0-168 is comprised between about 22940 mcg·h/mL and about 28480 mcg·h/mL at week 1; AUC 0-168 is comprised between about 50000 mcg·h/mL and about 65000 mcg·h/mL at week 4, e.g. comprised between about 56040 mcg·h/mL and about 61080 mcg·h/mL at week 4; AUC 0-168 is comprised between about 60000 mcg·h/mL and about 90000 mcg·h/mL at week 6, e.g. comprised between about 69110 mcg·h/mL and about 84630 mcg·h/mL at week 6. More in particular AUC 0-168 is selected from the group comprising about 20000 mcg·h/mL, about 25000 mcg·h/mL, about 30000 mcg·h/mL, about 35000 mcg·h/mL, about 40000 mcg·h/mL, about 45000 mcg·h/mL, about 50000 mcg·h/mL, about 55000 mcg·h/mL, about 60000 mcg·h/mL, about 65000 mcg·h/mL, about 70000 mcg·h/mL, about 75000 mcg·h/mL, about 80000 mcg·h/mL, about 85000 mcg·h/mL, about 90000 mcg·h/mL. The present invention also includes AUC 0-168 at any intermediate value of the above said values;

T1/2 is comprised between about 200 hours and about 500 hours, e.g. comprised between about 300 hours and about 450 hours, comprised between about 334 hour and about 445 hours at week 6. In particular t1/2 is selected from the group comprising about 200 hour, about 250 hour, about 300 hours, about 350 hours, about 370 hours, about 400 hours, about 450 hours, about 500 hours. The present invention also includes t1/2 at any intermediate value of the above said values;

CLss is comprised between about 5 mL/h and about 20 mL/h. In particular CLss is comprised between about 7 mL/h and about 20 mL/h at week 4 e.g. CLss is comprised between about 11.8 mL/h and about 17.9 mL/h at week 4; CLss is comprised between about 5 mL/h and about 15 mL/h at week 6, e.g. comprised between about 9.35 mL/h and about 12.2 mL/h at week 6. More in particular CLss is selected from the group comprising about 5 mL/h, about 8 mL/h, about 10 mL/h, about 12 mL/h, about 15 mL/h. The present invention also includes CL at any intermediate value of the above said values;

Vz is comprised between about 3 L and about 9 L, e.g. comprised between about 4 L and about 7 L, comprised between about 5.57 L and about 6 L at week 6. In particular Vz is selected from the group comprising about 3 L, about 4 L, about 5 L, about 6 L, about 7 L, about 8 L, about 9 L. The present invention also includes Vz at any intermediate value of the above said values;

Rac is comprised between about 1 mcg·h/mL and about 5 mcg·h/mL. In particular Rac is comprised between about 1 mcg·h/mL and about 3 mcg·h/mL at week 4 e.g. Rac is comprised between about 2.10 mcg·h/mL and about 2.60 mcg·h/mL at week 4; Rac is comprised between about 2 mcg·h/mL and about 4 mcg·h/mL at week 6, e.g. comprised between about 2.40 mcg·h/mL and about 3.40 mcg·h/mL at week 6. More in particular Rac is selected from the group comprising about 1 mcg·h/mL, about 1.5 mcg·h/mL, about 2 mcg·h/mL, about 2.5 mcg·h/mL, about 3 mcg·h/mL, about 3.5 mcg·h/mL, about 4 mcg·h/mL, about 4.5 mcg·h/mL, about 5 mcg·h/mL. The present invention also includes CL at any intermediate value of the above said values;

Cavg is comprised between about 200 mcg/mL and about 600 mcg/mL. In particular Cavg is comprised between about 300 mcg/mL and about 400 mcg/mL at week 4 e.g. comprised between about 334 mcg/mL and about 364 mcg/mL at week 4; Cavg is comprised between about 400 mcg/mL and about 550 mcg/mL at week 6, e.g. comprised between about 411 mcg/mL and about 504 mcg/mL at week 6. More in particular Cavg is selected from the group comprising about 200 mcg/mL, about 250 mcg/mL, about 300 mcg/mL, about 350 mcg/mL, about 400 mcg/mL, about 450 mcg/mL, about 500 mcg/mL, about 550 mcg/mL, about 600 mcg/mL. The present invention also includes CL at any intermediate value of the above said values;

C trough is comprised between about 100 mcg/mL and about 400 mcg/mL. In particular C trough is comprised between about 200 mcg/mL and about 300 mcg/mL at week 4 e.g. comprised between about 229 mcg/mL and about 283 mcg/mL at week 4; C trough is comprised between about 250 mcg/mL and about 450 mcg/mL at week 6, e.g. comprised between about 307 mcg/mL and about 391 mcg/mL at week 6. More in particular C trough is selected from the group comprising about 100 mcg/mL, about 150 mcg/mL, 200 mcg/mL, about 250 mcg/mL, about 300 mcg/mL, about 350 mcg/mL, about 400 mcg/mL. The present invention also includes CL at any intermediate value of the above said values;

In certain embodiment, when the antibody of the present invention is administered intravenously at multiple doses of about 20 mg/kg of a subject body weight, for at least 6 consecutive weeks at least once a week, pharmacokinetics parameters may have the following values:

Cmax is comprised between about 300 mcg/mL and about 1500 mcg/mL. In particular Cmax is comprised between about 400 mcg/mL and about 700 mcg/mL at week 1, e.g. comprised between about 474 mcg/mL and about 619 mcg/mL at week 1; Cmax is comprised between about 900 mcg/mL and about 1300 mcg/mL at week 4, e.g. comprised between about 965 mcg/mL and about 1220 mcg/mL at week 4; Cmax is comprised between about 1000 mcg/mL and about 1400 mcg/mL at week 6, e.g. comprised between about 1020 mcg/mL and about 1340 mcg/mL at week 6. More in particular, Cmax is selected from the group comprising about 300 mcg/mL, about 350 mcg/mL, about 400 mcg/mL, about 450 mcg/mL, about 500 mcg/mL, about 550 mcg/mL, about 600 mcg/mL, about 650 mcg/mL, about 700 mcg/mL, about 750 mcg/mL, about 800 mcg/mL, about 850 mcg/mL, about 900 mcg/mL, about 950 mcg/mL, about 1000 mcg/mL, about 1100 mcg/mL, about 1200 mcg/mL, about 1300 mcg/mL, about 1400 mcg/mL, about 1500 mcg/mL. The present invention also includes Cmax at any intermediate value of the above said values;

Tmax is comprised between about 30 min and about 12 hours. In particular, tmax is comprised between about 30 min and about 5 hours at week 1 e.g. comprised between about 1.53 hour and about 4.02 hours at week 1; tmax is comprised between about 30 min and about 10 hours at week 4 e.g. comprised between about 1.00 hour and about 8 hours at week 4; tmax is comprised between about 30 min and about 3 hours at week 6 e.g. comprised between about 1.00 hour and about 1.52 hours at week 6. More in particular tmax is selected from the group comprising about 30 min, about 1 hour, about 1.5 hour, about 2 hours, about 3 hours, about 4 hours, about 5 hours, about 6 hours, about 7 hours, about 8 hours, about 9 hours, about 10 hours. The present invention also includes tmax at any intermediate value of the above said values;

AUC 0-168 is comprised between about 40000 mcg·h/mL and about 180000 mcg·h/mL. In particular AUC 0-168 is comprised between about 45000 mcg·h/mL and about 65000 mcg·h/mL at week 1, e.g. AUC 0-168 is comprised between about 50310 mcg·h/mL and about 61000 mcg·h/mL at week 1; AUC 0-168 is comprised between about 120000 mcg·h/mL and about 150000 mcg·h/mL at week 4, e.g. comprised between about 123800 mcg·h/mL and about 145000 mcg·h/mL at week 4; AUC 0-168 is comprised between about 14000 mcg·h/mL and about 180000 mcg·h/mL at week 6, e.g. comprised between about 143000 mcg·h/mL and about 171800 mcg·h/mL at week 6. More in particular AUC 0-168 is selected from the group comprising about 40000 mcg·h/mL, about 45000 mcg·h/mL, about 50000 mcg·h/mL, about 55000 mcg·h/mL, about 60000 mcg·h/mL, about 65000 mcg·h/mL, about 70000 mcg·h/mL, about 75000 mcg·h/mL, about 80000 mcg·h/mL, about 85000 mcg·h/mL, about 90000 mcg·h/mL, about 100000 mcg·h/mL, about 110000 mcg·h/mL, about 120000 mcg·h/mL, about 130000 mcg·h/mL, about 140000 mcg·h/mL, about 150000 mcg·h/mL, about 160000 mcg·h/mL, about 170000 mcg·h/mL, about 180000 mcg·h/mL. The present invention also includes AUC 0-168 at any intermediate value of the above said values;

T1/2 is comprised between about 200 hours and about 600 hours, e.g. comprised between about 300 hours and about 550 hours, comprised between about 385 hour and about 544 hours at week 6. In particular t1/2 is selected from the group comprising about 200 hour, about 250 hour, about 300 hours, about 350 hours, about 370 hours, about 400 hours, about 450 hours, about 500 hours, about 550 hours, about 600 hours. The present invention also includes t1/2 at any intermediate value of the above said values;

CLss is comprised between about 5 mL/h and about 20 mL/h. In particular CLss is comprised between about 6 mL/h and about 18 mL/h at week 4 e.g. CLss is comprised between about 7.94 mL/h and about 15.9 mL/h at week 4; CLss is comprised between about 5 mL/h and about 15 mL/h at week 6, e.g. comprised between about 6.86 mL/h and about 13.8 mL/h at week 6. More in particular CLss is selected from the group comprising about 5 mL/h, about 8 mL/h, about 10 mL/h, about 12 mL/h, about 15 mL/h. The present invention also includes CL at any intermediate value of the above said values;

Vz is comprised between about 2 L and about 12 L, e.g. comprised between about 3 L and about 10 L, comprised between about 4.20 L and about 8.85 L at week 6. In particular Vz is selected from the group comprising about 2 L, about 3 L, about 4 L, about 5 L, about 6 L, about 7 L, about 8 L, about 9 L, about 10 L, about 11 L, about 12 L. The present invention also includes Vz at any intermediate value of the above said values;

Rac is comprised between about 1 mcg·h/mL and about 5 mcg·h/mL. In particular Rac is comprised between about 1 mcg·h/mL and about 3 mcg·h/mL at week 4 e.g. Rac is comprised between about 2.20 mcg·h/mL and about 2.60 mcg·h/mL at week 4; Rac is comprised between about 2 mcg·h/mL and about 4 mcg·h/mL at week 6, e.g. comprised between about 2.70 mcg·h/mL and about 3.00 mcg·h/mL at week 6. More in particular Rac is selected from the group comprising about 1 mcg·h/mL, about 1.5 mcg·h/mL, about 2 mcg·h/mL, about 2.5 mcg·h/mL, about 3 mcg·h/mL, about 3.5 mcg·h/mL, about 4 mcg·h/mL, about 4.5 mcg·h/mL, about 5 mcg·h/mL. The present invention also includes CL at any intermediate value of the above said values;

Cavg is comprised between about 600 mcg/mL and about 1200 mcg/mL. In particular Cavg is comprised between about 700 mcg/mL and about 900 mcg/mL at week 4 e.g. comprised between about 737 mcg/mL and about 863 mcg/mL at week 4; Cavg is comprised between about 800 mcg/mL and about 1100 mcg/mL at week 6, e.g. comprised between about 851 mcg/mL and about 1023 mcg/mL at week 6. More in particular Cavg is selected from the group comprising about 600 mcg/mL, about 700 mcg/mL, about 750 mcg/mL, about 800 mcg/mL, about 850 mcg/mL, about 900 mcg/mL, about 950 mcg/mL, about 1000 mcg/mL, about 1050 mcg/mL, about 1100 mcg/mL, about 1200 mcg/mL. The present invention also includes CL at any intermediate value of the above said values;

C trough is comprised between about 400 mcg/mL and about 1100 mcg/mL. In particular C trough is comprised between about 500 mcg/mL and about 800 mcg/mL at week 4 e.g. comprised between about 568 mcg/mL and about 748 mcg/mL at week 4; C trough is comprised between about 600 mcg/mL and about 1000 mcg/mL at week 6, e.g. comprised between about 657 mcg/mL and about 936 mcg/mL at week 6. More in particular C trough is selected from the group comprising about 400 mcg/mL, about 450 mcg/mL, 300 mcg/mL, about 350 mcg/mL, about 400 mcg/mL, about 450 mcg/mL, about 500 mcg/mL, about 550 mcg/mL, 600 mcg/mL, about 650 mcg/mL, about 700 mcg/mL, about 750 mcg/mL, about 800 mcg/mL, about 850 mcg/mL, 900 mcg/mL, about 950 mcg/mL, about 1000 mcg/mL, about 1050 mcg/mL, about 1100 mcg/mL. The present invention also includes CL at any intermediate value of the above said values;

In another embodiment, the antibody of the present invention is administrated subcutaneously at a single dose comprised between about 20 mg and about 1.5 g, e.g. comprised between about 50 mg and about 1 g, between about 60 mg and about 800 mg, between about 70 mg and about 600 mg. In a particular embodiment, the antibody of the present invention is administrated intravenously at a dose equal to or less than 1.5 g. In a more particular embodiment, the antibody of the present invention is administrated at a dose selected from the group comprising about 20 mg, about 50 mg, about 75 mg, about 100 mg, about 200 mg, about 300 mg, about 400 mg, about 500 mg, about 600 mg, about 700 mg, about 800 mg, about 1 g, about 1.5 g. The present invention also includes administration doses at any intermediate value of the above said values.

In certain embodiments, when the antibody of the present invention is administered subcutaneously at a single dose of about 600 mg, pharmacokinetics parameters may have the following values:

Cmax is comprised between about 20 μg/mL and about 100 μg/mL, e.g. comprised between about 25 μg/mL and about 95 μg/mL, comprised between about 30 μg/mL and about 90 μg/mL. In particular Cmax is selected from the group comprising about 20 μg/mL, about 30 μg/mL, about 50 μg/mL, about 70 μg/mL, about 90 μg/mL, about 100 μg/mL. The present invention also includes Cmax at any intermediate value of the above said values;

Tmax is comprised between about 20 hours and about 250 hours, e.g. comprised between about 30 hours and about 220 hours, comprised between about 40 hours and about 200 hours. In particular Tmax is selected from the group comprising about 20 hours, about 40 hours, about 70 hours, about 100 hours, about 120 hours, about 150 hours, about 180 hours, about 200 hours, about 220 hours, about 250 hours. The present invention also includes Tmax at any intermediate value of the above said values;

AUC 0-168 is comprised between about 3000 μg*h/mL and about 14000 μg*h/mL, e.g. comprised between about 3500 μg*h/mL and about 13500 μg*h/mL, comprised between about 3700 μg*h/mL and about 13000 μg*h/mL. In particular AUC 0-168 is selected from the group comprising about 3000 μg*h/mL, about 5000 μg*h/mL, about 7000 μg*h/mL, about 10000 μg*h/mL, about 10500 μg*h/mL, about 12000 μg*h/mL, about 13000 μg*h/mL, about 14000 μg*h/mL. The present invention also includes AUC 0-168 at any intermediate value of the above said values;

AUC 0-last is comprised between about 17000 μg*h/mL and about 56000 μg*h/mL, e.g. comprised between about 18000 μg*h/mL and about 55000 μg*h/mL, comprised between about 19000 μg*h/mL and about 54700 μg*h/mL. In particular AUC 0-last is selected from the group comprising about 17000 μg*h/mL, about 20000 μg*h/mL, about 30000 μg*h/mL, about 40000 μg*h/mL, about 50000 μg*h/mL, about 60000 μg*h/mL. The present invention also includes AUC 0-last at any intermediate value of the above said values;

AUC 0-infinity is comprised between about 17000 μg*h/mL and about 60000 μg*h/mL, e.g. comprised between about 18000 μg*h/mL and about 58000 μg*h/mL, comprised between about 19500 μg*h/mL and about 57500 μg*h/mL. In particular AUC 0-infinity is selected from the group comprising about 17000 μg*h/mL, about 30000 μg*h/mL, about 40000 μg*h/mL, about 50000 μg*h/mL, about 60000 μg*h/mL. The present invention also includes AUC 0-infinity at any intermediate value of the above said values;

CL is comprised between about 7 mL/h and about 40 mL/h, e.g. comprised between about 8 mL/h and about 35 mL/h, comprised between about 10 mL/h and about 33 mL/h, comprised between about 10.5 mL/h and about 30.5 mL/h. In particular CL is selected from the group comprising, about 7 mL/h, about 15 mL/h, about 30 mL/h, about 40 mL/h. The present invention also includes CL at any intermediate value of the above said values;

Vz is comprised between about 3 L and about 15 L, e.g. comprised between about 5 L and about 14 L. In particular Vz is selected from the group comprising about 3 L, 5 L, 7 L, 10 L, 12 L, 14 L, 15 L. The present invention also includes Vz at any intermediate value of the above said values;

t1/2 is comprised between about 100 hours and about 600 hours, e.g. comprised between about 200 hours and about 500 hours, comprised between about 250 hour and about 450 hours. In particular t1/2 is selected from the group comprising about 100 hours, 150 hours, about 200 hour, about 250 hours, about 300 hours, 350 about, 400 hours, about 450 hours, about 500 hours, about 550 hours, about 600 hours. The present invention also includes t1/2 at any intermediate value of the above said values;

In certain embodiments, when the antibody of the present invention is administered subcutaneously at a single dose of about 75 mg, pharmacokinetics parameters may have the following values:

Cmax is comprised between about 1 μg/mL and about 20 μg/mL, e.g. comprised between about 2 μg/mL and about 18 μg/mL. In particular Cmax is selected from the group comprising about 1 μg/mL, about 2 μg/mL, about 5 μg/mL, about 7 μg/mL, about 10 μg/mL, about 12 μg/mL, about 15 μg/mL, about 17 μg/mL, about 20 μg/mL. The present invention also includes Cmax at any intermediate value of the above said values;

Tmax is comprised between about 20 hours and about 250 hours, e.g. comprised between about 30 hours and about 220 hours, comprised between about 40 hours and about 200 hours. In particular Tmax is selected from the group comprising about 20 hours, about 40 hours, about 70 hours, about 100 hours, about 120 hours, about 150 hours, about 180 hours, about 200 hours, about 220 hours, about 250 hours. The present invention also includes Tmax at any intermediate value of the above said values;

AUC 0-168 is comprised between about 300 μg*h/mL and about 2500 μg*h/mL, e.g. comprised between about 350 μg*h/mL and about 2400 μg*h/mL, comprised between about 380 μg*h/mL and about 2350 μg*h/mL. In particular AUC 0-168 is selected from the group comprising about 300 μg*h/mL, about 500 μg*h/mL, about 700 μg*h/mL, about 1000 μg*h/mL, about 1300 μg*h/mL, about 1500 μg*h/mL, about 1700 μg*h/mL, about 2000 μg*h/mL, about 2300 μg*h/mL, about 2500 μg*h/mL. The present invention also includes AUC 0-168 at any intermediate value of the above said values;

AUC 0-last is comprised between about 1300 μg*h/mL and about 10000 μg*h/mL, e.g. comprised between about 1500 μg*h/mL and about 9000 μg*h/mL. In particular AUC 0-last is selected from the group comprising about 1000 μg*h/mL, about 2000 μg*h/mL, about 3000 μg*h/mL, about 4000 μg*h/mL, about 5000 μg*h/mL, about 6000 μg*h/mL, about 7000 μg*h/mL, about 8000 μg*h/mL, about 9000 μg*h/mL, about 10000 μg*h/mL. The present invention also includes AUC 0-last at any intermediate value of the above said values;

AUC 0-infinity is comprised between about 1500 μg*h/mL and about 10000 μg*h/mL, e.g. comprised between about 1600 μg*h/mL and about 9000 μg*h/mL. In particular AUC 0-infinity is selected from the group comprising about 1000 μg*h/mL, about 2000 μg*h/mL, about 3000 μg*h/mL, about 4000 μg*h/mL, about 5000 μg*h/mL, about 6000 μg*h/mL, about 7000 μg*h/mL, about 8000 μg*h/mL, about 9000 μg*h/mL, about 10000 μg*h/mL. The present invention also includes AUC 0-infinity at any intermediate value of the above said values;

CL is comprised between about 5 mL/h and about 60 mL/h, e.g. comprised between about 8 mL/h and about 50 mL/h. In particular CL is selected from the group comprising about 5 mL/h, about 10 mL/h, about 15 mL/h, about 20 mL/h, about 30 mL/h, about 40 mL/h, about 50 mL/h, about 60 mL/h. The present invention also includes CL at any intermediate value of the above said values;

Vz is comprised between about 1 L and about 30 L, e.g. comprised between about 2 L and about 20 L. In particular Vz is selected from the group comprising about 1 L, 5 L, 10 L, 20 L, 30 L. The present invention also includes Vz at any intermediate value of the above said values;

t1/2 is comprised between about 100 hours and about 400 hours, e.g. comprised between about 150 hours and about 360 hours. In particular t1/2 is selected from the group comprising about 100 hours, about 150 hour, about 200 hours, about 250 hours, 300 about, 350 hours, about 400 hours. The present invention also includes t1/2 at any intermediate value of the above said values.

In a further embodiment of the present invention, the disclosed antibody is administered subcutaneously at loading dose comprised between about 50 mg and about 2 g on Day 1, followed by at least one maintenance dose comprised between about 20 mg and about 1 g, starting on a day comprised between Day 10 and Day 40.

In another embodiment, the antibody of the present invention is administered subcutaneously at a dose comprised between about 50 mg and about 2 g and/or at a dose comprised between about 20 mg and about 1 g.

According to one aspect of the present invention, the antibody of the present invention is administered subcutaneously the loading dose comprised between about 50 mg and about 2 g, or between about 100 mg and about 1.5 g, or between about 150 mg and about 1.2 g, or between about 150 mg and about 600 g. More specifically the loading dose is at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 450 mg, or at least 500 mg, or at least 550 mg, or at least 600 mg, or at least 650 mg, or at least 700 mg, or at least 750 mg, or at least 800 mg, or at least 850 mg, or at least 900 mg, or at least 950 mg, or at least 1 g, or at least 1.2 g, or at least 1.5 g. Even more specifically the loading dose is selected from the group comprising about 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 1.2 g, 1.5 g and 2g. The present invention also includes loading doses at any intermediate value between the above stated doses.

According to another aspect of the present invention, the maintenance dose is comprised between about 20 mg and about 1 g, or between about 50 mg and about 800 mg, or between about 70 mg and about 600 mg, or between about 70 mg and about 300 mg. More specifically the loading dose is at least 20 mg, or at least 30 mg, or at least 40 mg, or at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 450 mg, or at least 500 mg, or at least 550 mg, or at least 600 mg, or at least 700 mg, or at least 750 mg, or at least 800 mg, or at least 850 mg, or at least 900 mg, or at least 950 mg, or at least 1 g. Even more specifically the loading dose is selected from the group comprising about 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g. The present invention also includes loading and maintenance doses at intervals of 1, 5, and 10 mg between the above stated doses. The present invention also includes loading doses at any intermediate value between the above stated doses.

According to one aspect of the present invention, the loading dose is administered at Day 1.

According to another aspect of the present invention, the maintenance dose is administered starting on a day subsequent to Day 1. In certain embodiments, the maintenance dose is administered starting on a day comprised between about Day 2 and about Day 90. In a more preferred embodiment the maintenance dose is administered starting on a day comprised between about Day 10 and about Day 40. In particular the maintenance dose is administered starting on a day selected from the group comprising Day 2, Day 8, Day 15, Day 22, Day 29, Day 36, Day 43, Day 50, Day 57, Day 64, Day 71, Day 78, Day 85, Day 92. In a specific embodiment the maintenance dose is administered starting on Day 15, or on Day 29. The present invention also includes that the maintenance dose is administered starting on 1, 2, 3, 4, 5, 6, 7, 8, 9, 10 day(s) subsequent to the above stated starting days.

According to another aspect of the present invention, the maintenance dose is administered every n days after the starting day, wherein n is comprised between about 1 day and about 90 days. More preferably n is comprised between about 10 day and about 40 days. In particular n is at least 1 day, at least 7 days, at least 14 days, at least 21 days, at least 28 days, at least 35 days, at least 42 days, at least 49 days, at least 56 days, at least 63 days, at least 70 days, at least 77 days, at least 84 days, at least 91 days. More specifically, n is selected from the group comprising 1 day, 7 days, 14 days, 21 days, 28 days, 35 days, 42 days, 49 days, 56 days, 63 days, 70 days, 77 days, 84 days, 91 days. In a preferred embodiment n is selected from the group comprising 15 days and 30 days. The present invention also includes that n at intervals of 1, 2, 3, 4, 5, 6, 7, 8, 9, 10 day(s) subsequent to the above stated n days.

In particular, the present invention provides an anti-OX40 antagonist antibody for use in the treatment of OX40-mediated disorders, wherein said antibody is administrated
(i) at loading dose comprised between about 300 mg and about 1 g on Day 1, followed by at least one maintenance dose comprised between about 100 mg and about 600 mg, starting on a day comprised between Day 10 and Day 20
(ii) at loading dose comprised between about 300 mg and about 1 g on Day 1, followed by at least one maintenance dose comprised between about 100 mg and about 600 mg, starting on a day comprised between Day 20 and Day 40
(iii) at loading dose comprised between about 50 mg and about 300 mg on Day 1, followed by at least one maintenance dose comprised between about 20 mg and about 150 mg, starting on a day comprised between Day 20 and Day 40
(iv) at loading dose comprised between about 800 mg and about 1.5 g on Day 1, followed by at least one maintenance dose comprised between about 300 mg and about 800 mg, starting on a day comprised between Day 10 and Day 20

In a particular aspect, the maintenance dose is administrated every n days after the loading dose, wherein n is: comprised between 10 days and 20 days; or comprised between 20 days and 40 days.

More specifically, the present invention provides an anti-OX40 antagonist antibody for use in the treatment of OX40-mediated disorders, wherein said antibody is administrated
(i) at loading dose of about 600 mg on Day 1, followed by at least one maintenance a dose of about 300 mg; or
(ii) at loading dose of about 600 mg on Day 1, followed by at least one maintenance a dose of about 300 mg; or
(iii) at loading dose of about 150 mg on Day 1, followed by at least one maintenance a dose of about 75 mg.
(iv) at loading dose of about 1.2 g on Day 1, followed by at least one maintenance a dose of about 600 mg.

Even more specifically, the present invention provides an anti-OX40 antagonist antibody for use in the treatment of OX40-mediated disorders, wherein said antibody is administrated
(i) at loading dose of about 600 mg on Day 1, followed by a maintenance dose of about 300 mg starting at Day 15 every 2 weeks; or
(ii) at loading dose of about 600 mg on Day 1, followed by a maintenance dose of about 300 mg starting at Day 29 every 4 weeks; or
(iii) at loading dose of about 150 mg on Day 1, followed by a maintenance dose of about 75 mg starting at Day 29 every 4 weeks.
(iv) at loading dose of about 1.2 g on Day 1, followed by a maintenance dose of about 600 mg starting at Day 15 every 2 weeks; or

Also provided by the present disclosure is a method for treating an OX40 mediated disorder by administering to a patient
(i) at loading dose comprised between about 300 mg and about 1 g on Day 1, followed by at least one maintenance dose comprised between about 100 mg and about 600 mg, starting on a day comprised between Day 10 and Day 20
(ii) at loading dose comprised between about 300 mg and about 1 g on Day 1, followed by at least one maintenance dose comprised between about 100 mg and about 600 mg, starting on a day comprised between Day 20 and Day 40
(iii) at loading dose comprised between about 50 mg and about 300 mg on Day 1, followed by at least one maintenance dose comprised between about 20 mg and about 150 mg, starting on a day comprised between Day 20 and Day 40
(iv) at loading dose comprised between about 800 mg and about 1.5 g on Day 1, followed by at least one maintenance dose comprised between about 300 mg and about 800 mg, starting on a day comprised between Day 10 and Day 20

wherein the maintenance dose is administrated every n days after the loading dose, wherein n is comprised between 10 days and 20 days; or comprised between 20 days and 40 days.

More specifically, also provided by the present disclosure is a method for treating an OX40 mediated disorder by administering to a patient
(i) at loading dose of about 600 mg on Day 1, followed by at least one maintenance a dose of about 300 mg; or
(ii) at loading dose of about 600 mg on Day 1, followed by at least one maintenance a dose of about 300 mg; or
(iii) at loading dose of about 150 mg on Day 1, followed by at least one maintenance a dose of about 75 mg.
(iv) at loading dose of about 1.2 g on Day 1, followed by at least one maintenance a dose of about 600 mg;

Even more specifically, the present disclosure also provides a method for treating an OX40 mediated disorder by administering to a patient
(i) at loading dose of about 600 mg on Day 1, followed by a maintenance dose of about 300 mg starting at Day 15 every 2 weeks; or
(ii) at loading dose of about 600 mg on Day 1, followed by a maintenance dose of about 300 mg starting at Day 29 every 4 weeks; or
(iii) at loading dose of about 150 mg on Day 1, followed by a maintenance dose of about 75 mg starting at Day 29 every 4 weeks.
(iv) at loading dose of about 1.2 g on Day 1, followed by a maintenance dose of about 600 mg starting at Day 15 every 2 weeks.

In a specific embodiment, each subject receives a dose on Day 1 (2 injections) and every 2 weeks (q2w) (1 injection per occasion) starting from Day 15 through Week 14, according to the following treatment assignment below:
Group 1: Dose of 600 mg GBR 830 (2 SC injections each containing 300 mg in a 2 mL volume) on Day 1, followed by q2w dosing of 300 mg GBR 830 (1 SC injection containing 300 mg in a 2 mL volume), starting at Day 15 (Week 2).
Group 2: Dose of 600 mg GBR 830 (2 SC injections each containing 300 mg in a 2 mL volume) on Day 1, followed by dosing every 4 weeks (q4w) of 300 mg GBR 830 (1 SC injection containing 300 mg in a 2 mL volume) starting at Day 29. In order to maintain blinding, placebo (1 SC injection of 2 mL) is administered q4w starting at Day 15 (Week 2).
Group 3: Dose of 150 mg GBR 830 (2 SC injections each containing 75 mg in a 2 mL volume) on Day 1, followed by q4w dosing of 75 mg GBR 830 (1 SC injection containing 75 mg in a 2 mL volume) starting at Day 29. In order to maintain blinding, placebo (1 SC injection of 2 mL) is administered q4w starting at Day 15 (Week 2).

In another particular embodiment, each subject receives a dose on Day 1 (2 injections) and every 2 weeks (q2w) (1 injection per occasion) starting from Day 15 through Week 14, according to the following treatment assignment below:
Group 1: Subcutaneous (SC) administration of GBR830 as a loading dose of 600 mg (2×2 ml injections of a 150 mg/mL formulation) on day 1, followed by maintenance dose of 300 mg (1×2 injection of 150 mg/mL formulation) every 2 weeks.
Group 2: Subcutaneous (SC) administration of GBR830 as a loading dose of 600 mg (2×2 ml injections of a 150 mg/mL formulation) on day 1, followed by maintenance dose of 300 mg (1×2 injection of 150 mg/mL formulation) every 4 weeks. In order to maintain blinding, placebo (1 SC injection of 2 mL) is administrated q4w starting at Day 15 (Week 2)
Group 3: Subcutaneous (SC) administration of GBR830 as a loading dose of 150 mg (2×2 ml injections of a 37.5 mg/mL formulation) on day 1, followed by maintenance dose of 75 mg (1×2 injection of 37.5 mg/mL formulation) every 4 weeks. In order to maintain blinding, placebo (1 SC injection of 2 mL) is administrated q4w starting at Day 15 (Week 2)

As used herein, the term “OX40-mediated disorder” includes conditions such as allergy, asthma, COPD, rheumatoid arthritis, psoriasis and diseases associated with autoimmunity and inflammation. In particular, according to the present invention, exemplary OX40 mediated disorders include infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

Other exemplary OX40 mediated disorder include infections (viral, bacterial, fungal and parasitic), endotoxic shock associated with infection, arthritis, rheumatoid arthritis, asthma, bronchitis, influenza, respiratory syncytial virus, pneumonia, chronic obstructive pulmonary disease (COPD), idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

In accordance to a preferred aspect of the present invention, the anti-OX40 antagonist antibody is used for the treatment or prevention of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

The present invention also provides a method for treating an OX40-mediated disorder, wherein the OX40-mediated disorder is selected from the group comprising atopic dermatitis, rheumatoid arthritis, autoimmune uveitis, multiple sclerosis, lupus (such as systemic lupus erythematosus) and graft-versus-host disease (GVHD), scleroderma, hidradenitis, and ulcerative colitis.

In one embodiment of the present invention, the anti-OX40 antagonist antibody is GBR 830 (CAS Registry Number 2126777-87-3).

In a more specific embodiment of the present invention, the OX40-mediate disorder is atopic dermatitis, wherein atopic dermatitis is mild, or mild-to-moderate, or moderate, or moderate-to-severe, or severe. In an even more specific embodiment, OX40-mediate disorder is moderate-to-severe atopic dermatitis.

Atopic dermatitis, Atopic dermatitis” (AD), as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present invention encompasses methods to treat patients with mild, moderate-to-severe or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 10% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art.

The present invention provides materials and methods for improving one or more Atopic dermatitis efficacy parameter(s) in a subject. Examples of “AD related efficacy parameters” include: (a) Scoring of Atopic Dermatitis-SCORAD (b) Investigators Global Assessment (IGA); (c) Pruritus Numerical rating scale (NRS) (d) Dermatology Life Quality Index-DLQI (e) Body Surface Area (BSA); (f) Eczema Area and Severity Index (EASI); (h) and trans-epidermal water loss (TEWL). An “improvement in an AD-related efficacy parameters” means a decrease from baseline of one or more of IGA, BSA, EASI, SCORAD, TEWL, DLQI or NRS.

Investigators Global Assessment (IGA): the IGA is an assessment scale used in clinical studies to determine severity of AD and clinical response to treatment based on a 5-point scale ranging from 0 (clear) to 4 (severe/very severe):

0=Clear: No inflammatory signs of atopic dermatitis

1=Almost clear: Just perceptible erythema, and just perceptible papulation/infiltration. Barely perceptible erythema and/or minimal lesion elevation (papulation/infiltration)

2=Mild disease: Mild erythema and mild papulation/infiltration. Visibly detectable, light pink erythema and very slight elevation (papulation/infiltration)

3=Moderate disease: Moderate erythema and moderate papulation/infiltration. Dull red, clearly distinguishable erythema; clearly perceptible elevation (papulation/infiltration), but not extensive

4=Severe disease: Severe erythema and severe papulation/infiltration. Deep/dark red erythema; marked and extensive elevation (papulation/infiltration)

The Eczema Area and Severity Index (EASI) is a validated measure used in clinical practice and clinical studies to assess the severity and extent of AD. Four AD disease characteristics will be assessed for severity by the investigator or designee on a scale of “0” (absent) through “3” (severe). In addition, the area of AD involvement will be assessed as a percentage by body area of head/neck, trunk (including genital area), upper limbs, and lower limbs (including buttocks), and converted to a score of 0 to 6 (Hanifin et al, 2001).

The SCORing Atopic Dermatitis Assessment (SCORAD) is a validated tool used in clinical research and clinical practice that was developed to standardize the evaluation of the extent and intensity of AD. The extent of AD is assessed as a percentage of each defined body area and reported as the sum of all areas, with a maximum score of 100% (assigned as “A” in the overall SCORAD calculation). The intensity of 6 specific symptoms of AD is assessed using the following scale: absence (0), mild (1), moderate (2), or severe (3), (for a maximum of 18 total points, assigned as “B” in the overall SCORAD calculation). Subjective assessment of pruritus and sleeplessness is recorded for each symptom by the subject or relative on a visual analogue scale (VAS), where 0 is no pruritus (or sleeploss) and 10 is the worst imaginable pruritus (or sleeploss), with a maximum possible score of 20. This parameter is assigned as “C” in the overall SCORAD calculation. The SCORAD is calculated as: A/5+7B/2+C (Kunz et al, 1997).

For the Pruritus Numerical Rating Scale (Pruritus NRS), subjects will respond to the following question, “On a scale of 0-10, with 0 being no itch and 10 being the worst itch imaginable, how would you rate your worst degree of itch during the previous 24 hours?” The NRS will be assessed and recorded by the subject once per day, in the morning ideally at the same time, and reviewed by study staff at each clinic visit. Baseline Pruritus NRS average score for maximum itch intensity will be determined based on the average of daily NRS scores for maximum itch intensity (the daily score ranges from 0 to 10) during the 7 days immediately preceding) randomization. A minimum of 3 daily scores out of the 7 days is required to calculate the baseline average score. For subjects who do not have at least 4 daily scores reported during the 7 days immediately preceding the planned randomization date, randomization should be postponed until this requirement is met, but without exceeding the 28-day maximum duration for screening).

The Dermatology Life Quality Index (DLQI) is a subject-administered, 10-question, validated, quality-of-life questionnaire that covers 6 domains including symptoms and feelings, daily activities, leisure, work and school, personal relationships, and treatment. Response categories include “a little,” “a lot,” and “very much” with corresponding scores of 1, 2, and 3, respectively; “not at all”, “not relevant” responses are scored as “0.” Totals range from 0 to 30 (i.e., from less to more impairment) and a 5-point change from baseline is considered clinically relevant (Finlay and Khan, 1994; Basra et al, 2008).

For the Global Individual Signs Score (GISS), individual components of the AD lesions (erythema, infiltration/papulation, excoriations, and lichenification) will be rated globally (i.e., each assessed for the whole body, not by anatomical region) on a 4-point scale (from 0=none to 3=severe) using the EASI severity grading criteria.

Body surface area (BSA) affected by AD will be assessed for each section of the body (the possible highest score for each region is: head and neck [9%], anterior trunk [18%], back [18%], upper limbs [18%], lower limbs [36%], and genitals [1%]) and will be reported as a percentage of all major body sections combined.

The Hospital Anxiety Depression Scale (HADS) is an instrument for screening anxiety and depression in non-psychiatric populations; repeated administration also provides information about changes to a patient's emotional state (Zigmond and Snaith, 1983; Herrmann, 1997). The HADS consists of 14 items, 7 each for anxiety and depression symptoms; possible scores range from 0 to 21 for each subscale. The following cut-off scores are recommended for both subscales: 7 to 8 for possible presence, 10 to 11 for probable presence, and 14 to 15 for severe anxiety or depression.

The Patient-Oriented Eczema Measure (POEM) is a 7-item, validated questionnaire used in clinical practice and clinical trials to assess disease symptoms in children and adults (Charman et al, 2004). The format is a response to 7 items (dryness, itching, flaking, cracking, sleep loss, bleeding, and weeping) based on frequency during the past week (i.e., 0=no days, 1=1 to 2 days, 2=3 to 4 days, 3=5 to 6 days, and 4=all days) with a scoring system of 0 to 28; the total score reflects disease-related morbidity.

The EuroQol-5D (EQ-5D) is a standardized measure of health status developed by the EuroQOL Group in order to provide a simple, generic measure of health for clinical and economic appraisal. The EQ-5D consists of 2 parts: the descriptive system and the EQ visual analogue scale (EQVAS). The EQ-5D descriptive system comprises the following 5 dimensions: mobility, self-care, usual activities, pain/discomfort, and anxiety/depression. Each dimension has 3 levels of perceived problems: “no problems” (level 1), “some problems” (level 2), “extreme problems” (level 3). The VAS scale is a 100-point scale with endpoints ranging from 100-“best imaginable health state” to 0—“worst imaginable health state”.

The Asthma Control Questionnaire-5 (ACQ-5) is a 5-question version of the Juniper ACQ is a validated questionnaire to evaluate asthma control. The questionnaire will be administered only to the subset of subjects with a medical history of asthma.

The Sino-nasal Outcome Test (SNOT-22) is a validated questionnaire to assess the impact of chronic rhinosinusitis on quality of life (QOL). The questionnaire will be administered only to the subset of subjects with chronic inflammatory conditions of the nasal mucosa and/or paranasal sinuses (e.g., chronic rhinitis/rhinosinusitis, nasal polyps, allergic rhinitis).

Patient Global Assessment of Disease: subjects will rate their overall wellbeing based on a 5-point Likert scale from poor to excellent. Subjects will be asked: “Considering all the ways in which your eczema affects you, indicate how well you are doing.” Response choices are: “Poor”; “Fair”; “Good”; “Very Good”; “Excellent.”

Patient Global Assessment of Treatment: subjects will rate their satisfaction with the study treatment based on a 5-point Likert scale from poor to excellent. Subjects will be asked: “How would you rate the way your eczema responded to the study medication?” Response choices are: “Poor”; “Fair”; “Good”; “Very Good”; “Excellent”.

Atopic dermatitis biomarker parameters. The present invention also includes methods involving the use, quantification, and analysis of Atopic dermatitis biomarker parameters. As used herein, the term “Atopic dermatitis biomarker parameters” means any biological response, cell type, parameter, protein, polypeptide, enzyme, enzyme activity, metabolite, nucleic acid, carbohydrate, or other biomolecule which is present or detectable in an AD patient at a level or amount that is different from (e.g., greater than or less than) the level or amount of the marker present or detectable in a non-AD patient. In some embodiments, the term “Atopic dermatitis biomarker parameters” includes a biomarker associated with Type 2 helper T-cell Th2)-driven inflammation. In order to evaluate for the drug effect or how much of the disease profile has been reversed by treatment as measured changes in the AD transcriptome using gene arrays consisting of differentially expressed genes between lesional and non lesional AD skin as defined by fold changes (typically a fold change of more than 2). The AD disease phenotype is the integration of cellular and molecular markers that define the epidermal pathology (hyperplasia, differentiation abnormalities), and Th2, and Th22 immune activation. The changes or reversal of these immune and barrier defects will be assessed by IHC and RT-PCR.

Other exemplary AD-associated biomarkers include a panel of Th1, Th2, Th22, Th17/Th22 cytokines and chemokines e.g., K16, Ki67, IFNγ, CXCL10, IL-31, IL-4, IL-13, CCL11, CCL17, TSLPR, IL-23p19, IL-8, and S100As, Serum Thymus and activation-regulated chemokine (TARC/CCL17), eotaxin-3, total Immunoglobulin E (IgE), Thymus and activation-regulated chemokine is a chemokine, shown to be strongly associated with disease severity in AD, and may be involved in pathogenesis of the disease. Baseline TARC levels will be assessed for potential predictive value for treatment response. Eotaxin-3 (CCL26), Eotaxin-3 is a chemokine, shown to be associated with disease severity in AD, and may be involved in pathogenesis of the disease. Baseline eotaxin-3 levels will be assessed for potential predictive value for treatment response. Post-treatment samples will be evaluated for effects of anti OX40 antagonist antibody on eotaxin-3. Total Immunoglobulin E (IgE), Patients with AD often have elevated IgE. Total IgE levels have been found to modestly correlate with AD severity and may be involved in the pathogenesis of the disease. Changes in total IgE reflects not only on AD, but atopy in general. Baseline IgE levels will be assessed for potential predictive value for treatment response. Trans-epidermal water loss (TEWL). Transepidermal water loss is a skin barrier function test that measures perspiration or water loss through the skin. This procedure involves the non-invasive application of a probe on the surface of the skin on the arm or leg. Affected and non-affected areas of skin will be tested.

The present invention also relates to a stable pharmaceutical formulation comprising the disclosed antibody. In one embodiment of the present invention, the stable pharmaceutical formulation comprising the disclosed antibody for IV infusion comprises about 10 mg/ml GBR830, about 15 mM Histidine, about 150 mM NaCl, and about 0.01% Tween 80 and has a pH of about 6.25. In another embodiment, the stable pharmaceutical formulation comprising the disclosed antibody for SC administration comprises from about 150 to about 250 mg of GBR 830, about 160 mg of sucrose, about 3.1 mg of histidine, and about 0.4 mg of polysorbate 80, and is designed to deliver about 150 mg of GBR 830 in about 1.0 mL injection after reconstitution with about 1.1 mL of sterile water for injection.

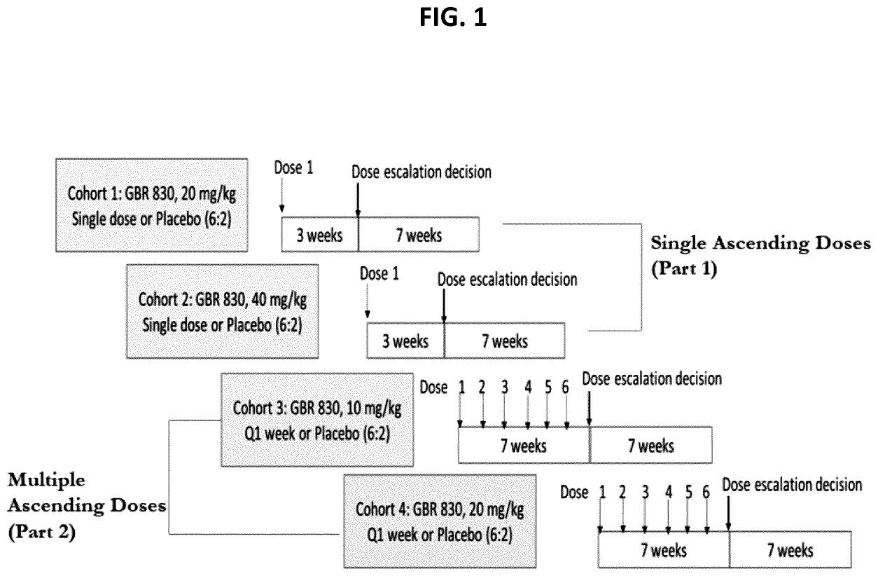

FIG. 1. Study design. Q1: once per interval (week)

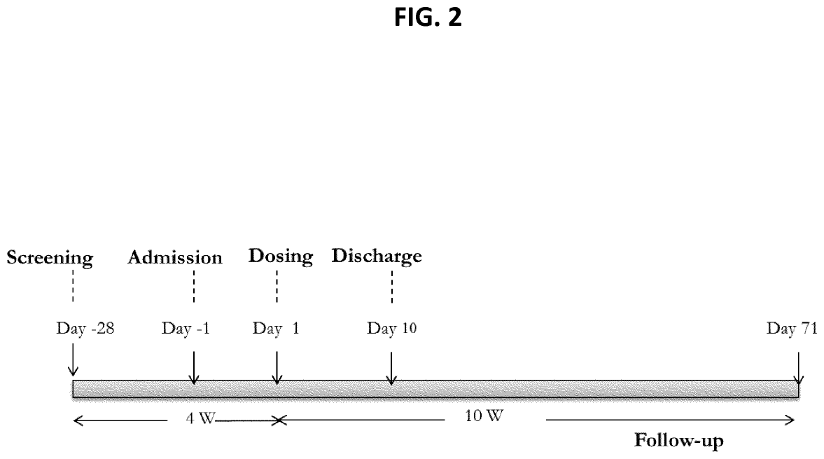

FIG. 2. Study design schematic

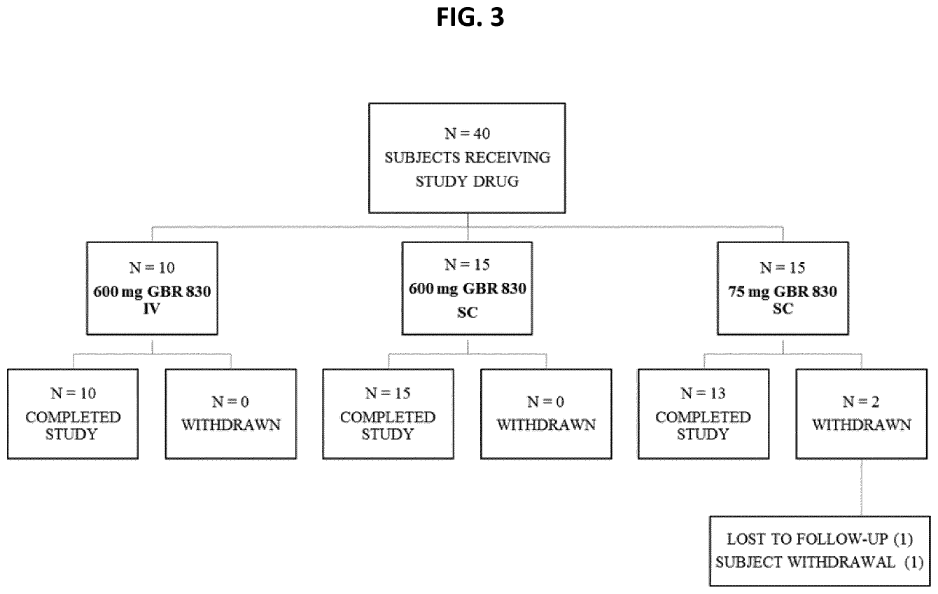

FIG. 3. Disposition of Subjects

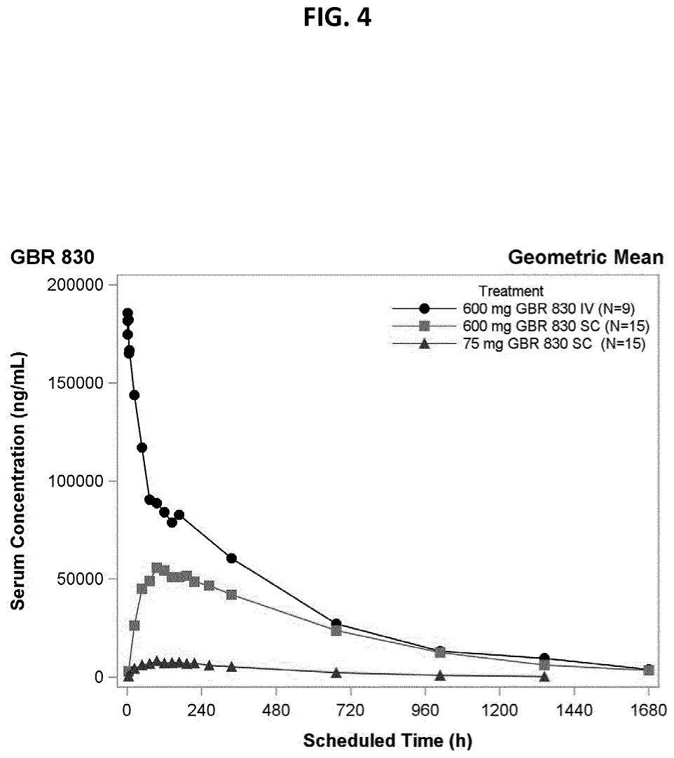

FIG. 4. Geometric Mean PK Serum Concentration-Time Profiles (Linear Scale; PKAS)

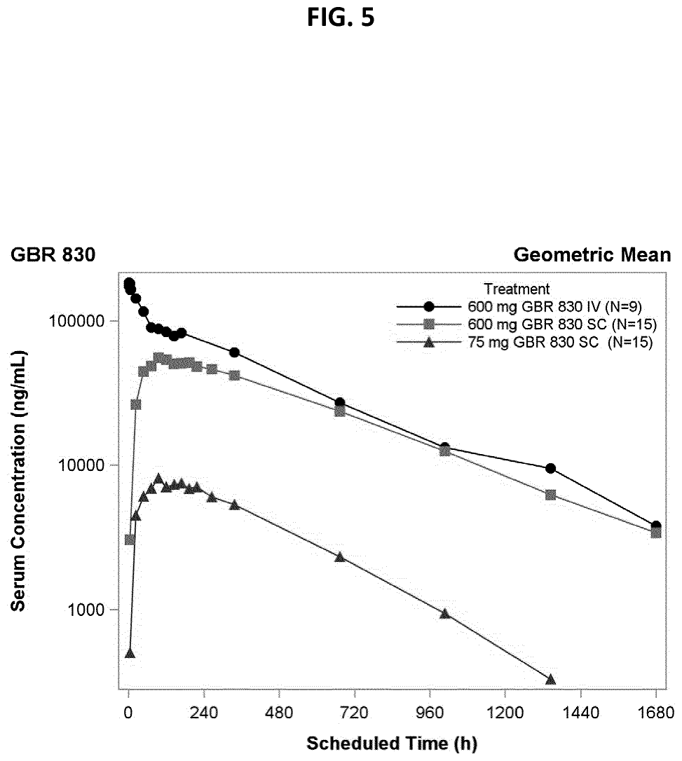

FIG. 5. Geometric Mean PK Serum Concentration-Time Profiles (Semi-Logarithmic Scale: PKAS)

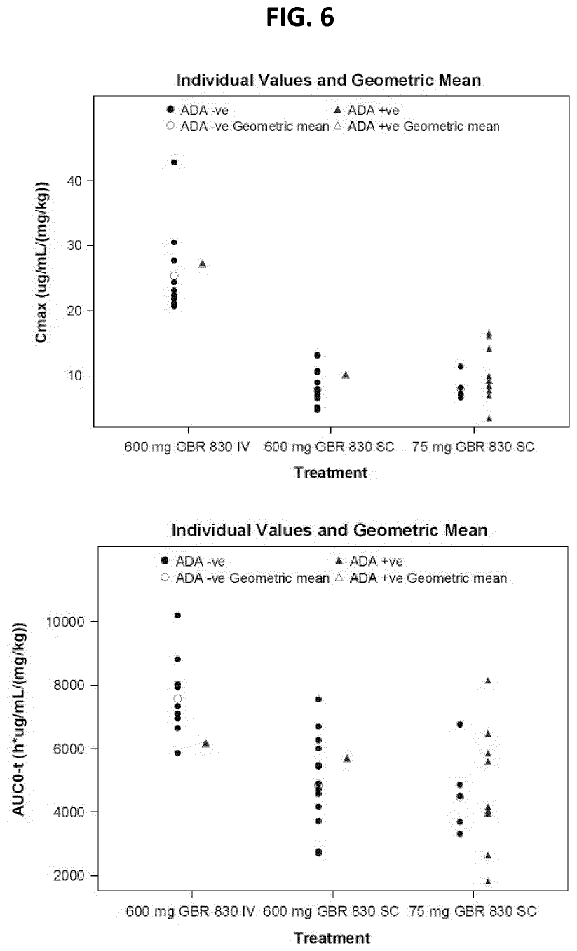

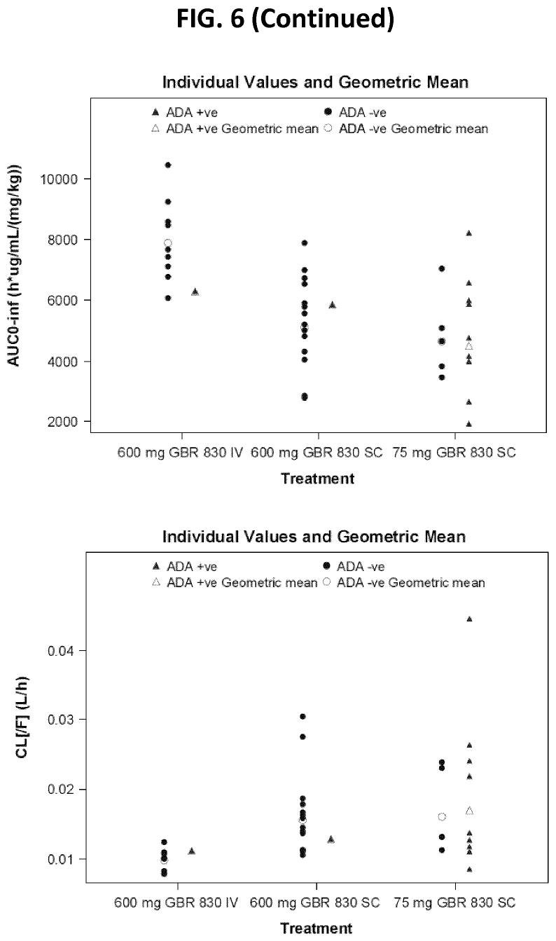

FIG. 6. Impact of ADA on the PK Parameters of GBR 830

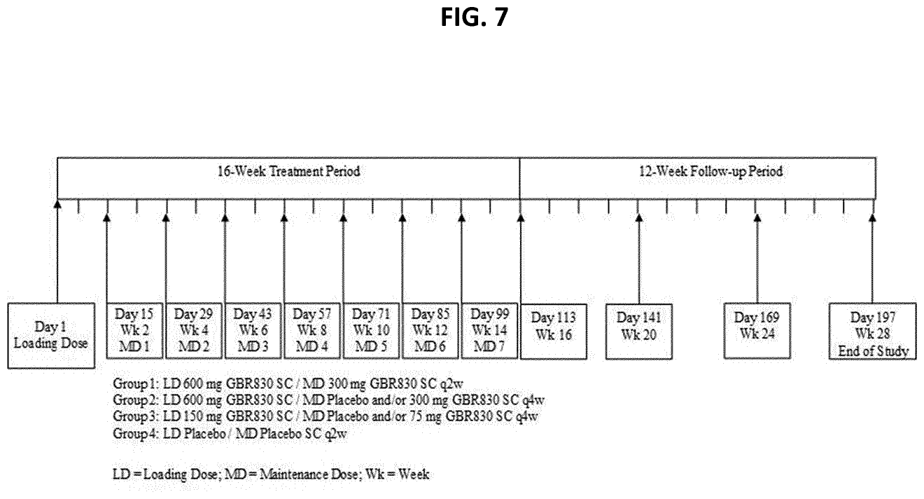

FIG. 7. Study design.

### EXAMPLES

### Example 1. Randomized 2-Part, Phase 1, Randomized, Single-Blind, Placebo-Controlled, Single Ascending Dose (SAD, Part 1) and Multiple Ascending Dose (MAD, Part 2) Study

Study Objectives and Purpose

The purpose of this phase 1 study is to explore safety, tolerability, PK, and immunogenicity of GBR830 after single and multiple intravenous (IV) infusion.

The primary objective of the study is:
Safety and tolerability of single and multiple doses of GBR830

The secondary objectives of the study are:
PK of single and multiple doses of GBR830
Immunogenicity of GBR830

The exploratory/other objectives of the study are:
Biological response biomarkers

Overall Study Design

This is a 2-part, phase 1, randomized, single-blind, placebo-controlled, single ascending dose (SAD, Part 1) and multiple ascending dose (MAD, Part 2) study and each cohort is conducted in 3 periods: screening, treatment, and follow-up. The study is intended to evaluate the safety, tolerability, PK, and immunogenicity of GBR830 in adult healthy subjects after administration of increasing doses of GBR830.

Four (4) cohorts of subjects (8 per cohort) are randomized to receive either GBR830 or placebo in a 6:2 ratio (6 GBR830: 2 placebo). In Part 1 (SAD), subjects will receive a single dose administration (20 mg/kg or 40 mg/kg in separate cohorts). In Part 2 (MAD), subjects will receive multiple dose administration (10 mg/kg or 20 mg/kg in separate cohorts, administered weekly for 6 consecutive weeks, see FIG. 1).

Intermediate or lower dose levels may be used instead of the planned dose levels. The dose may be escalated in smaller increments, if, in the opinion of the Dose Escalation Team (DET), it is deemed appropriate with respect to emerging safety data. In no case will the dose administered to any subject exceed 40 mg/kg. Based on the safety, tolerability and PK data after repeated administration of 20 mg/kg/week in Part 2, the Sponsor may decide to test a higher dose level up to 40 mg/kg/week through a protocol amendment. The decision to escalate the dose will be determined by an assigned DET using dose escalation criteria. The DET will be led by the Principal Investigator (PI) of the study. Additional team members are the Sponsor's Clinical Lead, the Safety/Pharmacovigilance Representative, the Medical Monitor, and the PK Representative. Additional members may be assigned by the Sponsor, based on need. Based on assessment of the data, the PI will provide a dose escalation report including the decision whether or not to proceed with dose escalation. The report will include a listing of the Adverse Events (AEs) and any clinically significant abnormalities in vital signs, laboratory tests and electrocardiograms (ECGs) and PK data.

Screening (All Subjects)

Screening will occur between Day −28 and Day −2. The purpose of screening is to obtain informed consent and to establish protocol eligibility. Informed consent will be obtained after the study has been fully explained to each subject and before the conduct of any screening procedures or assessments. The Screening Disposition electronic case report form (eCRF) page must be completed to indicate whether the subject is eligible to participate in the study and to provide reasons for screen failure, if applicable. Subjects who fail to satisfy inclusion/exclusion criteria may be rescreened once for the study within the screening period based on PI's discretion.

Treatment Phase

Part 1

If eligible for participation in the study, subjects will be admitted to the Clinical Research Unit (CRU) on Day −1 (Visit 2) for eligibility checks (confirmation of inclusion/exclusion criteria). They will be dosed in the morning of Day 1 (Visit 2) and will remain in-house until Day 3 for the 48 hours post-dose assessments. On Day 3, the Investigator will check all subjects' well-being prior to their discharge from the CRU. If necessary, subjects will remain at the CRU until any AEs causing concern have resolved. At the day of discharge (Day 3), each subject will be given a safety card to carry at all times in case of any emergency occurring outside of the CRU. The card gives details of the study number, the start and end date of subject's involvement in the study, subject details, name of the responsible physician and the address and telephone number of the CRU.

Part 2

If eligible for participation in the study, subjects will be admitted to the CRU on Day −1 of Dosing 1 (Visit 2), for eligibility checks. They will be dosed in the morning of Day 1 (Visit 2) and will remain in-house until Day 2 for the 24 hours post-dose assessments. On Day 2, the Investigator will check all subjects' well-being prior to their discharge from the CRU. If necessary, subjects will remain at the CRU until any AEs causing concern have resolved. At the day of discharge (Day 2), each subject will be given a safety card to carry at all times in case of any emergency occurring outside of the CRU. The card gives details of the study number, the start and end date of subject's involvement in the study, subject details, name of the responsible physician and the address and telephone number of the CRU. Subjects will return to the CRU weekly for 6 weeks. The procedure described above will be repeated for Dosing 2 through 6 (i.e., Visit 5, Visit 6, Visit 7, Visit 10, and Visit 11).

Follow-Up

Part 1

Subjects will be observed for 10 weeks following administration of the investigational product (IP) to characterize the safety, tolerability, and PK profile of GBR830. All subjects will return for outpatient visits and for a final end of study visit on Day 71±2 (Visit 10). The end of the study will be the date of the last study visit for the last subject in the study. Any subject experiencing an AE will be followed until resolution of the AE.

Part 2

Subjects will be observed for 7 weeks following the last administration of study drug to characterize the safety, tolerability, and PK profile of GBR830. Following Dosing 1, 4, and 6, subjects will return to the CRU for outpatient visits on Day 3, Day 5, Day 24, Day 26, and Day 38 (Visit 3, Visit 4, Visit 8, Visit 9, and Visit 12, respectively). All subjects will return for a final end of study visit on Day 92±2 (Visit 19). The end of the study will be the date of the last study visit for the last subject in the study. Any subject experiencing an AE will be followed until resolution of the AE.

Rationale for Study Design and Doses

The rationale for this combined SAD/MAD study is to evaluate the safety, tolerability, PK, and immunogenicity of GBR830 at different doses of GBR830 after a single IV infusion in a phase 1 SAD study. In addition, the safety, tolerability, PK, and immunogenicity of GBR830 will be evaluated following repeat IV infusions administered once weekly for 6 consecutive weeks.

The study will be conducted in 2 parts; Part 1 and Part 2.

Part 1 is a SAD study, where 2 dose levels (20 mg/kg and 40 mg/kg), higher than previously evaluated (10 mg/kg), will be explored in 2 separate cohorts, each consisting of 8 subjects (6:2, GBR830: placebo). The dose escalation is planned conservatively, with only 2-fold dose increments. The first dose level in Part 1 is planned to be 20 mg/kg, 2-fold higher than the previously evaluated dose level. Part 2 will be initiated after review of the safety, tolerability and PK data from Part 1. The proposed first dose level in Part 2 is 10 mg/kg/week for 6 weeks. Based on the safety, tolerability and PK data after repeated administration of 20 mg/kg/week for 6 weeks in Part 2, the Sponsor may decide to test a higher dose level up to 40 mg/kg/week through a protocol amendment. In each SAD cohort, a sentinel group consisting of 2 subjects randomized in a ratio of 1:1 to GBR830: placebo will be dosed initially. After at least 2 days have elapsed, 6 subjects randomized in ratio of 5:1 to GBR830: placebo will be dosed at the same dose level. The PK of GBR830 over a 1 week duration and safety over a 3 week duration will be assessed before the dose escalation to the next cohort. Based on the available PK data at the previous cohort, the serum exposure expected at the next higher dose would be projected. The dose escalation to the next planned dose will be commenced only if no dose-limiting toxicity (DLT) is observed in the previous cohort and the projected serum exposure is less than the PK based dose escalation stopping criteria.

Part 2 is a MAD study, consisting of 2 cohorts with 8 subjects in each cohort (6:2, GBR830: placebo). In this part, 2 dose levels that were safe and well tolerated after single dose administration will be administered once every week for 6 consecutive weeks. The proposed first dose in Part 2 is 10 mg/kg/week for 6 weeks, and will be commenced only after evaluating at least 1 week PK and 3 week safety data following single IV administration at 20 and 40 mg/kg (Part 1). The dose escalation to 20 mg/kg/week is planned conservatively, with only 2 fold increments in Part 2. The first MAD dose level (10 mg/kg/week) is 10-fold lower than the NOAEL (no observed adverse effect level) dose (100 mg/kg/week) established in the 6-week preclinical toxicology study in monkeys and is anticipated to give approximately 5-fold lower steady state serum exposure (Cmax and AUC0-lweek) than that was achieved at the NOAEL dose in the 6-week toxicology study in cynomolgus monkeys. The dose escalation to the next higher dose level will commence only after assessing the steady state PK (after 4th dose) and safety at the previous dose level. Based on the available PK data at the previous cohort, the steady state serum exposure expected at the next higher dose will be projected. The dose escalation to the next planned dose will be commenced only if no DLT is observed in the previous cohort and the projected serum exposure is less than the PK based dose escalation stopping criteria.

Estimated Duration of Subject Participation and Timing for the Study

The expected total duration of subject participation in the study is up to 99 days in Part 1 and 120 days in Part 2. In Part 1, this duration includes the screening period (28 days), study duration of 71 days, including an in-house stay of 4 days and 10 week follow-up period. In Part 2, this duration includes the screening period (28 days), study duration of 92 days, including once weekly dosing for 6 weeks (with 3 in-house study days for each dosing, for a total of 18 in-house days) and a 7 week follow-up period (following the last administration of study drug). The interval between screening and dosing for all subjects should not exceed 28 days.

Number of Subjects

Up to 32 subjects in 4 cohorts of 8 subject each (6 GBT830:2 placebo)

Selection and Withdrawal of Subjects

Subject Inclusion Criteria
1. Provision of valid signed and dated written informed consent and any locally required authorization prior to any protocol-related procedures, including screening evaluations.
2. Male or female subjects, aged ≥18 years to ≤65 years at the time of informed consent.
3. At screening, subject must have a body mass index (BMI) of 18.5 to 32.0 kg/m² (inclusive); weight must be >50 kg.
4. Subjects must be willing and able to understand and comply with all aspects of the protocol.
5. Must be willing to use a highly effective form of contraception, e.g., double barrier method, for the duration of the study. Methods like periodic abstinence, post ovulation procedures and withdrawal are not considered adequate. If female and of childbearing potential, she must have a negative serum pregnancy test result within 7 days prior to first dosing and a negative pregnancy test on Day −1. Each female will be considered to have childbearing potential unless surgically sterilized by hysterectomy or has been post menopausal for at least 2 years.
6. Non-smokers or ex-smokers who have not smoked for a period of at least 3 months or non-users of nicotine-containing products for a period of at least 3 months.
7. Subjects who are otherwise healthy and free from illness or disease as determined by medical history, vital signs, physical examinations, ECG, laboratory studies, and/or other tests performed within 28 days prior to drug administration, as judged by the Investigator.

Subject Exclusion Criteria
1. Subjects with a history of hypersensitivity to murine proteins or any drug or other allergies, which are considered, in the opinion of the Investigator, to contraindicate study participation.
2. Subjects who have received a live vaccination within 12 weeks prior to randomization, or who intend to receive a live vaccination during the course of the study, or have participated in a vaccine clinical study within 12 weeks prior to randomization.
3. Subjects who are immunocompromised (congenital or acquired), or who have had a recent (within 3 months prior to randomization) or current serious systemic or local infection (including infectious mononucleosis-like illness or herpes zoster).
4. Subjects who have evidence of active or latent tuberculosis as documented in their medical history or test positive for peptide antigens that are associated with Mycobacterium tuberculosis infection, using the QuantiFERON®-TB Gold Plus test.
5. Subjects with a history of substance abuse or dependence that in the opinion of the Investigator is considered to interfere with the subject's participation in the study, or who test positive for drugs of abuse at screening or admission.
6. Subjects who are known to be seropositive for human immunodeficiency virus (HIV).
7. Subjects with a history of a positive result for Hepatitis B surface antigen (HBsAg), antibody to Hepatitis B core antigen (anti-HBcAg), or antibody to Hepatitis C virus (anti-HCV).
8. Subjects with an abnormal ECG at screening (including a QTc >450 msec) which is considered clinically significant in the opinion of the Investigator. (QTc calculated based on Fridericia formula).
9. Subjects with laboratory values, which are significantly different from normal reference ranges and/or judged to be clinically significant by the Investigator, including but not limited to:
   a. Serum creatinine >1.5 mg/dL (equivalent to SI unit of >114.39 μmol/L), blood urea nitrogen (BUN) ULN at screening.
   b. ALT or aspartate aminotransferase (AST) ≥2×ULN, and/or serum total bilirubin ≥1.5×ULN, at screening.
   c. Hemoglobin (Hb) value less than 11 g/dL (equivalent to SI unit of <6.83 mmol/L) at screening.
   d. Absolute neutrophil count ≤1500/μL (equivalent to SI unit of ≤1.5*10⁹/L) or absolute lymphocyte count ≤800/μL (equivalent to SI unit of ≤3.8*10⁹/L) or platelet count ≤100000/μL (equivalent to SI unit of ≤100*10⁹/L), or any abnormal evaluations judged clinically significant by the Investigator at screening and at pre-dose.
10. Subjects who have participated in a study with a new molecular entity or any other drug study within 3 months of IP administration.
11. Subjects with previous exposure to antibody therapies or administration of immunoglobulins (Ig) within 6 months of randomization.
12. Subjects with previous exposure to GBR830.
13. Subjects with a history of donating 1 unit of blood (450 mL) blood in the 3 months prior to IP administration or who intend to donate within 3 months of their last scheduled study visit.
14. Subjects with a history of hypertension or a supine blood pressure >150/90 mmHg at screening or a history of recurrent hypotensive events considered as clinically relevant or documented orthostatic hypotension or a supine blood pressure <100/45 mmHg at screening.
15. Subjects with an organ dysfunction, malignancy, and/or any illness.
16. Subjects who are currently taking or who have taken any prescription or non-prescription medication within 7 days of dosing including aspirin, dietary or mega dose vitamin supplements, and herbal preparations (except paracetamol and/or hormone replacement therapies).

Subject Withdrawal Criteria

A subject may voluntarily discontinue study participation at any time after giving informed consent and before the completion of the last visit of the study. Subjects may also be withdrawn from study drug treatment at the discretion of the Investigator or Sponsor for safety, noncompliance, or administrative reasons. The Investigator may also discontinue the subject's study participation at any time at his/her discretion and for any reason.

The reasons for subject withdrawal will be recorded and may include, but are not limited to:
1. Withdrawal of consent by the subject to continue in the study. If consent is withdrawn, the subject will not receive any further IP or further study observation. Note that the subject may need to undergo additional tests or tapering of treatment to withdraw safely.
2. Development of a serious or intolerable AE (Adverse Event) that necessitates discontinuation at the discretion of the Investigator (the AE section of the eCRF must be completed; AE includes SAE and death.
3. At the discretion of the Investigator, when he/she believes continued participation is not in the best interest of the subject.
4. At the discretion of the Investigator, when the subject does not adhere to the study procedures.
5. A positive pregnancy test.
6. A protocol deviation that, in the opinion of the Sponsor and Investigator, warrants discontinuation from the study.
7. Use of concomitant medications, which may interfere with the PK of the IP. Note: withdrawal in such cases will be discussed and mutually agreed by the Investigator and the Sponsor.

All efforts will be made to monitor the subjects withdrawn during the study for safety and PK analysis, throughout the duration of study. Subjects who are withdrawn may be replaced by a subject to receive the same treatment assignment as the original subject he/she is replacing.

Lost to Follow-Up

A subject will be considered lost-to-follow-up only if no contact has been established by the time the study is completed such that there is insufficient information to determine the subject's status on Visit 10/Day 71±2 (Part1) or Visit 19/Day 92±2 (Part 2). Subjects refusing to return to the site or to continue participation in the study should be documented as “withdrawal of consent” rather than “lost to follow-up.” Investigators should document attempts to re-establish contact with missing subjects throughout the study period. If contact with a missing subject is re-established, the subject should not be considered lost-to-follow-up and any evaluations should resume according to the protocol.

Permanent Discontinuation of Study Drug

A subject who is permanently discontinued from further receipt of study drug, regardless of the reason (withdrawal of consent, due to an AE, other), will be identified as having permanently discontinued treatment. Subjects who permanently discontinue treatment may either be considered to have completed the study or not to have completed the study. Subjects who are permanently discontinued from receiving IP will be followed for safety through the full study period (through Visit 10/Day 71±2 (Part 1) or Visit 19/Day 92±2 (Part 2), including the collection of any protocol-specified blood specimens, unless consent is withdrawn, the subject is lost to follow-up, or the subject is enrolled in another clinical study.

Replacement of Subjects

Subjects who are withdrawn may be replaced by a subject to receive the same treatment assignment as the original subject he/she is replacing.

Treatment of Subjects

Description of Study Drug

Table 1 summarizes the characteristics of the Investigational Product and of the Placebo.

TABLE 1
Study Drugs

| | Investigational Product | | |
|---|---|---|---|
| Product Name | GBR 830 | GBR 830 | GBR 830 |
| Dosage Form | Solution | Solution | Solution |
| Dosage | 10 mg/kg | 20 mg/kg | 40 mg/kg |
| Route of Administration | IV infusion | IV infusion | IV infusion |

| | Placebo |
|---|---|
| Product Name | GBR 830 placebo |
| Dosage Form | Solution |
| Dosage | contains no active ingredient |
| Route of Administration | IV |

Study Drug Materials and Management

Study Drug

Appropriate aseptic technique should be used while preparing and administering infusions. The study drug will be administered by continuous slow intravenal (IV) infusion over 60 minutes using a commercially available, validated, infusion pump. In the event of an infusion reaction, for the purpose of subject safety, the rate of infusion may be decreased and the duration extended at the Investigator's discretion. The pharmacist (or designee under the direction of the pharmacist) will dispense study drug for each subject according to the protocol and randomization list.

Investigational Product (IP)

GBR830 is provided as lyophilized powder; each vial is designed to deliver 150 mg/mL of GBR830 after reconstitution. GBR830 drug product is provided in 10 mL Type I glass vials as a buffered preservative free sterile solution for IV infusion after appropriate dilution. Each vial is designed to deliver 150 mg/mL GBR830 after reconstitution. Each vial also contains histidine, sucrose, and polysorbate 80 as excipients. The glass vials are equipped with a FluroTec®-coated rubber stopper and flip off cap.

Placebo

Commercially available saline bags will be used as placebo. These are the same bags used for the dilution of GBR830 drug product.

Solution for IV Infusion

The GBR830 solution for IV infusion will be prepared by diluting GBR830 drug product in sterile physiological 0.9% saline (commercially available normal saline [0.9% sodium chloride] provided by the CRO).

Other Clinical Supplies

Details of the infusion sets and any other material to be used will be described in a pharmacy manual.

Study Drug Storage

GBR830 vials must be stored refrigerated at +5° C.±3° C., protected from light and moisture and diluted within 24 h before IV administration in sterile physiological 0.9% saline (0.9% NaCl). Commercially available saline bags will be used as placebo and must be stored at the commercially labeled storage conditions.

Study Drug Preparation

The preparation of the infusion solutions (GBR830 and placebo) will be performed by a licensed pharmacist or trained designee under the direction of the Investigator. The IP will be diluted with sterile physiological 0.9% saline to yield a uniform solution for IV infusion. Foaming or excessive shearing of the protein solution must be avoided. The preparation must be carefully inspected; it should result in a homogeneous-looking clear solution free of visible particles. Direct sunlight should be avoided. The details of preparation and storage of the infusions are presented in a pharmacy manual. At the time of dispensing, a mandatory verification by a second staff member has to be performed.

Administration

The day of administration of the first dose of study drug is considered Day 1.

Appropriate aseptic technique should be used while preparing and administering infusions. GBR830 is provided as lyophilized powder; each vial is designed to deliver 150 mg/mL of GBR830 after reconstitution.

The IP will be diluted in sterile physiological 0.9% saline and administered after normalizing for body weight by continuous slow IV infusion over 60 minutes (±5 mins) using commercially available volumetric or syringe infusion pumps. The infusion is to be performed with the subject in a supine position and/or semi-supine position, with proper documentation noted in the medical records at the site.

The infusion volume must be calculated using the subject's current body weight. In the event of an infusion reaction, for the purposes of subject safety, the rate of infusion may be decreased and the duration extended at the Investigator's discretion. The pharmacist or designee under the direction of the Investigator will dispense study drug for each subject according to the protocol and the randomization number assigned through the randomization scheme. Details of the volume of IP required, the concentration to be made, the volume of final infusion to be administered, the infusion sets, and material to be used will be described in a pharmacy manual.

Pharmacokinetic, Biomarker, and Immunogenicity Assessments

Pharmacokinetic Assessments

Blood Sample Collection

Blood samples (3.5 mL each) will be collected as per routine phlebotomy procedures. The blood samples will be collected during the course of the study through indwelling cannula placed in forearm veins or by direct venipuncture from a site different from the IV infusion site. The exact times of blood sampling will be recorded in the eCRF. Actual time points will be used for PK calculations. The PK serum samples will be retained and may be used for analysis of OX40 or other biomarkers and results will be reported separately.

Sample Analysis

Serum concentrations of GBR830 will be quantified using a validated enzyme-linked immunosorbent assay (ELISA) method.

The GBR 830 enzyme immunoassay is a direct enzyme immunoassay. The antibody GBR 830 (IgG) in calibration standard samples, quality control samples and validation samples was captured by the antigen hOX40-His coated on a 96-well plate. Fcγ fragment specific peroxidase-conjugated Affinipure goat anti-human IgG antibody (AB_2337577) binds to GBR 830 immobilised on the plate by antigen. Upon addition of tetramethylbenzidine (TMB) substrate a blue colour was developed. Sulphuric acid was added to stop the reaction. The absorbance is measured at 450 nm (Ref. 620 nm).

Characteristics of the validated method are given below:
Calibrated Range: 0.977-62.5 ng/mL
Minimum Required Dilution (MRD): 1: 100
Defined LLOQ*: 97.7 ng/mL (0.977 ng/mL×100)
Defined ULOQ*: 6250 ng/mL (62.5 ng/mL×100)
Intra-assay Accuracy [bias %]: Between −15.3% and 3.21% and **between −13.0 and 1.15%
Intra-assay Precision [CV %]: (ICH: Repeatability) Between 1.88% and 5.74% and **between 2.59 and 9.21%
Inter-assay Accuracy [bias %]: Between −3.61% and 2.61% and **between −11.2 and 0.209%
Inter-assay Precision [CV %]: (ICH: Intermediate Precision) Between 5.50% and 13.1% and **between 3.84 and 10.8%
Total Error [TE %]: (Overall, inter- and intra-assay) Between 3.37% and 17.2% and **between 2.87 and 22.0%

*: Due to 100 fold dilution of validation samples the defined LLOQ and ULOQ values are 100 times higher than the measured concentration

Biomarker Assessments

Pharmacokinetic samples retained after PK analysis may be used for analysis of OX40, OX40L, or other biomarkers.

Immunogenicity Assessments

Blood samples (5 mL each) will be collected at appropriate time points defined previously, to detect anti-drug antibodies (ADAs) to GBR830, as per procedures similar to collection of PK samples.

Assessment of safety Safety assessments will consist of monitoring and recording all AEs and SAEs; regular monitoring of hematology, blood chemistry, and urine values; periodic measurement of vital signs and ECGs; and performance of physical examinations

Statistics

The statistical analysis will be coordinated by the responsible Sponsor biostatistician or designee at the Contract Research Organization [CRO]. The Statistical Analysis Plan (SAP) will be written to provide details of the analysis, along with specifications for tables, listings, and figures to be produced. The SAP will be finalized before the database lock at the latest. If there are differences, the information in the SAP will supersede the information in the protocol. Any changes from the analyses planned in the SAP will be justified in the CSR.

All analyses will be performed by the Sponsor (or designee CRO) using SAS® version 9.3 or above. In general, all data will be summarized with descriptive statistics (number of subjects, mean, and standard deviation [SD], minimum, median, and maximum) for continuous variables and frequency and percentage for categorical variables.

Sample Size

Assessment of the safety and tolerability of single and multiple doses of GBR 830 is the primary objective of this study. Thus, no formal sample size calculations have been performed for this study. The sample size of 32 subjects in 4 cohorts of 8 subjects each was chosen based on experience from previous studies of a similar nature.

Analysis Sets

Detailed criteria for analysis sets will be documented in the SAP and the allocation of subjects to analysis sets will be determined prior to database hard-lock.

Safety Analysis Set

The Safety Analysis Set (SAF) will consist of all subjects who have received at least 1 dose of the IP. This analysis set will be the primary analysis set for the safety end points.

Pharmacokinetic Analysis Set

The Pharmacokinetic Analysis Set (PKAS) consists of all subjects who are randomized, received at least 1 dose of study treatment, have sufficient assay results to permit calculation of the PK parameters, and who have no major protocol deviations that impact PK assessment. The PKAS will include only subjects treated with GBR 830.

Endpoints

Primary Endpoints
Frequency and severity of treatment-emergent adverse events (TEAEs) and SAEs for each treatment, based on CTCAE, version 4.03
Number of DLTs during treatment

Secondary Endpoints
Cmax, trough plasma concentration (Ctrough), time at which Cmax is observed (tmax), AUC to the end of the dosing period (AUC0-tau), AUC from time 0 to infinity (AUC0-∞), and AUC from time 0 until the last measurable concentration (AUC (0-t)), terminal elimination half life (t½), volume of distribution, clearance, and accumulation ratio (Rac) as applicable
ADA formation.

Exploratory Endpoints
Biological Response Biomarkers

Subject Disposition

Data on subject disposition (number of subjects enrolled, number of drop-outs, and reasons for drop-out), demographics (gender, age, height), and other baseline characteristics will be summarized. The safety, tolerability, PK, and other data from the study will be listed and summarized descriptively by treatment. The number (percentage) of subjects who were screened for the study (Enrolled Subjects, i.e., those who signed informed consent) and reasons for screen failure will be described.

Demographic and Other Baseline Characteristics

Demographics and other baseline characteristics will be summarized by treatment group for the SAF. Descriptive statistics will include number of subjects, mean, SD, minimum, median and maximum for continuous variables, and frequency and percentage for categorical variables.

Continuous demographic and baseline variables include age, height and body weight, and BMI; categorical variables include gender, race, and ethnicity.

Pharmacokinetic, Biomarker, and Immunogenicity Analyses

Pharmacokinetic Analyses

All PK analyses will be performed using the PKAS.

The PK parameters will be derived by non-compartmental analysis. Individual subject serum PK parameters will be determined from serum concentrations using non-compartmental methods for each subject using appropriate validated software.

Additional parameters may be evaluated depending on the data obtained during the study.

The derived PK parameters will be listed by subject and summarized by treatment. Wherever appropriate, data will be visualized by means of graphical representations. For the summaries, descriptive statistics for all relevant PK parameters will include: n, mean, SD, coefficient of variation (% CV), minimum, median, maximum, geometric mean, and geometric mean % CV. Details of the PK analyses will be provided in the SAP.

Biomarker Analyses

Summary statistics (n, mean, SD, median, min, max, geometric mean) of baseline and change from baseline will be provided for biomarkers by treatment.

Immunogenicity Analyses

Percent incidence, titer and neutralizing potential of ADA, if any, will be determined. Details of the analysis will be provided in the SAP.

Safety Analyses

All safety analyses will be performed on the SAF.

Extent of Exposure

Up to 32 subjects in 4 cohorts (with a placebo:active allocation ratio of 2:6) will be randomized to receive study drug. Exposure data will be listed.

Adverse Events

Adverse events will be coded using the most recent version of the Medical Dictionary for Regulatory Activities (MedDRA). The number and percentage of AEs and AEs related to study drug will be summarized by system organ class, preferred term and treatment group. The number and percentage of AEs by severity will also be summarized. All AEs will be displayed in listings. Serious adverse events and AEs leading to discontinuation will be displayed in listings.

Laboratory Values

For quantitative laboratory measurements descriptive statistics will be used to summarize results and change from baseline by treatment group and time point. Shifts in laboratory tests relative to normal ranges from baseline to post-baseline will also be tabulated. All laboratory data will be displayed in listings.

Vital Signs

Descriptive statistics will be used to summarize vital sign results and changes from baseline by treatment group and time. All vital signs data will be displayed in listings.

Electrocardiograms

All ECG variables will be presented by visit. Descriptive statistics for ECG parameters and changes from baseline will be presented by treatment.

Shift tables will present changes from baseline in ECG interpretation (categorized as normal; abnormal, not clinically significant; and abnormal, clinically significant) to end of treatment (or end of phase or by visit).

Physical Examination

All findings will be listed.

Interim Analysis

No interim analysis is planned for this study.

Dose Escalation Team

The DET will be led by the PI of the study. Additional team members are the Sponsor's Clinical Lead, the Safety/Pharmacovigilance Representative, the Medical Monitor, and the PK Representative. Additional members may be assigned by the Sponsor, based on need. Based on the assessment of the data, the PI will provide a dose escalation report including the decision whether or not to proceed with dose escalation (see Dose Escalation Criteria). The report will include a listing of the AEs and any clinically significant abnormalities in vital signs, laboratory tests, ECGs and PK data.

Results

Subject Disposition

A total of 32 healthy male and female subjects (22 Male and 10 Female) were included in the study. All the subjects were adults (18-64 years). The period 1 study information are reported in Table 2. The number of subjects in period 1 is reported in Table 3.

TABLE 2
Period 1 information
Period 1

| | |
|---|---|
| Period 1 title | Overall Study (overall period) |
| Is this the baseline | Yes |
| period? | |
| Allocation method | Randomized - controlled |
| Blinding used | Single blind |
| Roles blinded | Subject |

Arms

| Are arms mutually | Yes |
|---|---|
| exclusive? | |
| Arm title | GBR830, 20 mg/kg dose SAD [Part 1] |
| Arm description: | 6 Patients were randomized in this arm and |
| | received GBR830 at a dose of 20 mg/kg. |
| Arm type | Experimental |
| Investigational medicinal | GBR830 |
| Pharmaceutical forms | Solution for infusion |
| Routes of administration | Intravenous use |
| Dosage and administration | 20 mg/kg administered as IV Infusion |
| Arm title | GBR830, 40 mg/kg dose SAD [Part 1] |
| Arm description: | 6 Patients were randomized in this arm and |
| | received GBR830 at a dose of 40 mg/kg. |
| Arm type | Experimental |
| Investigational medicinal | GBR830 |
| Pharmaceutical forms | Solution for infusion |
| Routes of administration | Intravenous use |
| Dosage and administration | 40 mg/kg administered as IV Infusion |
| | over 60 minutes |
| Arm title | Placebo Arm SAD [Part 1] |
| Arm description: | 4 Subjects were randomized in this arm and |
| | received Placebo |
| Arm type | Placebo |
| Investigational medicinal | Placebo (sterile physiological 0.9% saline) |
| Pharmaceutical forms | Solution for infusion |
| Routes of administration | Intravenous use |
| Dosage and administration | Placebo administered as IV Infusion |
| details: | over 60 minutes |
| Arm title | GBR830, 10 mg/kg dose MAD [Part 2] |
| Arm description: | 6 Patients were randomized in this arm and |
| | received GBR830 at a dose of 10 mg/kg. |
| Arm type | Experimental |
| Investigational medicinal | GBR830 |
| Pharmaceutical forms | Solution for infusion |
| Routes of administration | Intravenous use |
| Dosage and administration | 10 mg/kg administered as an IV Infusion |
| | over 60 minutes |
| Arm title | GBR830, 20 mg/kg dose MAD [Part 2] |
| Arm description: | 6 subjects were randomized in this arm and |
| | received multiple dose [MD] of 20 mg/kg of |
| | GBR-830 |
| Arm type | Experimental |
| Investigational medicinal | GBR830 |
| Pharmaceutical forms | Solution for infusion |
| Routes of administration | Intravenous use |
| Dosage and administration | 20 mg/kg administered as IV Infusion |
| details: | over 60 minutes |
| Arm title | Placebo MAD [Part 2] |
| Arm description: | 4 subjects were randomized in this arm and |
| | received multiple dose [MD] of Placebo |
| Arm type | Placebo |
| Investigational medicinal | Placebo (sterile physiological 0.9% saline) |
| Pharmaceutical forms | Solution for infusion |
| Routes of administration | Intravenous use |
| Dosage and administration | Administered as IV Infusion over 60 minutes |
| details: | |

TABLE 3
Number of subjects in period 1

| | GBR830, | GBR830, | | GBR830, | GBR830, | |
|---|---|---|---|---|---|---|
| Number of | 20 mg/kg | 40 mg/kg | Placebo | 10 mg/kg | 20 mg/kg | Placebo |
| subjects in | dose SAD | dose SAD | Arm SAD | dose MAD | dose MAD | MAD |
| period 1 | [Part 1] | [Part 1] | [Part 1] | [Part 2] | [Part 2] | [Part 2] |
| Started | 6 | 6 | 4 | 6 | 6 | 4 |
| Completed | 6 | 6 | 3 | 5 | 6 | 4 |
| Not | 0 | 0 | 1 | 1 | 0 | 0 |
| completed | | | | | | |
| Consent | — | — | 1 | 1 | — | — |
| withdrawn | | | | | | |
| by subject | | | | | | |

Primary Endpoints

Treatment Emergent Adverse Events (TEAEs)

Treatment Emergent Adverse Events (TEAEs) were investigated as indicated in Table 4. The number of subjects with TEAEs are reported in Table 5.

TABLE 4
TEAEs
Primary: Treatment Emergent Adverse Events (TEAEs)

| | |
|---|---|
| End point title | Treatment Emergent Adverse Events (TEAEs) |
| End point type | Primary |
| End point | The total duration of Part-1 study was 99 days. |
| timeframe: | The total duration of Part-2 study was 120 days. |

TABLE 5
End point values

| | GBR830, | GBR830, | | GBR830, | GBR830, | |
|---|---|---|---|---|---|---|
| | 20 mg/kg | 40 mg/kg | Placebo | 10 mg/kg | 20 mg/kg | Placebo |
| End point | dose SAD | dose SAD | Arm SAD | dose MAD | dose MAD | MAD |
| values | [Part 1] | [Part 1] | [Part 1] | [Part 2] | [Part 2] | [Part 2] |
| Subject group | Reporting | Reporting | Reporting | Reporting | Reporting | Reporting |
| type | group | group | group | group | group | group |
| Number of | 6 | 6 | 4 | 6 | 6 | 4 |
| subjects | | | | | | |
| analysed | | | | | | |
| Units: Number | 5 | 5 | 2 | 5 | 5 | 4 |
| of subjects | | | | | | |
| with TEAEs | | | | | | |

Number of Dose Limiting Toxicities During Treatment

The number of dose limiting toxicities during treatment were investigated, as indicated in Table 6. The number of subjects with TEAEs are reported in Table 7.

TABLE 6
Number of dose limiting toxicities during treatment
Primary: Number of dose limiting toxicities during treatment

| | |
|---|---|
| End point title | Number of dose limiting toxicities during treatment |
| End point type | Primary |
| End point | The total duration of Part-1 study was 99 days. |
| timeframe: | The total duration of Part-2 study was 120 days. |

TABLE 7
End point values

| | GBR830, | GBR830, | | GBR830, | GBR830, | |
|---|---|---|---|---|---|---|
| | 20 mg/kg | 40 mg/kg | Placebo | 10 mg/kg | 20 mg/kg | Placebo |
| End point | dose SAD | dose SAD | Arm SAD | dose MAD | dose MAD | MAD |
| values | [Part 1] | [Part 1] | [Part 1] | [Part 2] | [Part 2] | [Part 2] |
| Subject group | Reporting | Reporting | Reporting | Reporting | Reporting | Reporting |
| type | group | group | group | group | group | group |
| Number of | 6 | 6 | 4 | 6 | 6 | 4 |
| subjects | | | | | | |
| analysed | | | | | | |
| Units: Number | 0 | 0 | 0 | 0 | 0 | 0 |
| of subjects | | | | | | |
| with TEAEs | | | | | | |

Secondary Endpoints: Pharmacokinetics—Part 1

The parameters monitored during GBR830 SAD (Part 1) pharmacokinetic studies include: Cmax (see Table 8 for end point information and Table 9 for the results); tmax (see Table 10 for end point information and Table 11 for the results); AUC 0-168 (see Table 12 for end point information and Table 13 for the results); AUC 0-last (see Table 14 for end point information and Table 15 for the results); AUC 0-infinity (see Table 16 for end point information and Table 17 for the results); t1/2 (see Table 18 for end point information and Table 19 for the results); CL (see Table 20 for end point information and Table 21 for the results); Vz (see Table 22 for end point information and Table 23 for the results); Vss (see Table 24 for end point information and Table 25 for the results).

Secondary: Pharmacokinetics of GBR830 SAD (Part 1)—Cmax

TABLE 8
End point information.

| | |
|---|---|
| End point | Pharmacokinetics of GBR830 SAD (Part 1) - Cmax^{[1]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[1]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point.

TABLE 9
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | mcg/mL | mcg/mL |
| geometric mean (full range | 574.0 (449 to 753) | 1113 (977 to 1290) |
| (min-max)) | | |

Secondary: Pharmacokinetics of GBR830-SAD (Part 1)—tmax

TABLE 10
End point information

| | |
|---|---|
| End point | Pharmacokinetics of GBR830- SAD (Part 1) -tmax^{[2]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[2]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 11
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: hour | hour | hour |
| median (full range (min-max)) | 1.5 (1.00 to 4.00) | 1.5 (1.07 to 1.55) |

Secondary: Pharmacokinetics of GBR830-SAD (Part 1)—AUC 0-168

TABLE 12
End point information

| | |
|---|---|
| End point | Pharmacokinetics of GBR830- SAD (Part 1) -AUC 0-168^{[3]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[3]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 13
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | mcg/mL | mcg/mL |
| geometric mean (full range | 50701 (44350 to | 108230 (90980 to |
| (min-max)) | 56530) | 120100) |

Secondary: Pharmacokinetics of GBR830-SAD (Part 1)—AUC 0-Last

TABLE 14
End point information

| | |
|---|---|
| End point | Pharmacokinetics of GBR830- SAD (Part 1) -AUC 0-last ^{[4]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[4]} The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 15
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | mcg/mL | mcg/mL |
| geometric mean (full range | 150630 (130400 to | 329930 (247300 to |
| (min-max)) | 178800) | 410200) |

Secondary: Pharmacokinetics of GBR830-SAD (Part 1)—AUC 0-infinity

TABLE 16
End point information

| | |
|---|---|
| End point | Pharmacokinetics of GBR830- SAD (Part 1) - |
| title | AUC 0-infinity^{[5]} |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[5]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 17
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | mcg/mL | mcg/mL |
| geometric mean (full range | 157910 (134100 to | 353620 (257800 to |
| (min-max)) | 190800) | 456300) |

Secondary: Pharmacokinetics of GBR830-SAD (Part 1)—t1/2

TABLE 18
End point information

| | |
|---|---|
| End point | Pharmacokinetics of GBR830- SAD (Part 1) -t½^{[6]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[6]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 19
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | hour | hour |
| geometric mean (full range | 372.1 (297 to 462) | 451 (352 to 527) |
| (min-max)) | | |

Secondary: Pharmacokinetics of GBR830-SAD (Part 1)—CL

TABLE 20
End point information

| | |
|---|---|
| End point | Pharmacokinetics of GBR830- SAD (Part 1) - CL^{[7]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[7]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 21
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | mL/h | mL/h |
| geometric mean (full range | 10.05 (8.34 to 13.7) | 8.626 (6.52 to 11.3) |
| (min-max)) | | |

Secondary: Pharmacokinetic Assessment of GBR830-SAD (Part 1)—Vz

TABLE 22
End point information

| | |
|---|---|
| End point | Pharmacokinetic Assessment of GBR830- SAD (Part 1)- Vz^{[8]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[8]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 23
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | litre(s) | litre(s) |
| geometric mean (full range | 5.397 (4.17 to 6.58) | 5.613 (4.64 to 6.98) |
| (min-max)) | | |

Secondary: Pharmacokinetics of GBR830-SAD (Part 1)−Vss

TABLE 24
End point information

| | |
|---|---|
| End point | Pharmacokinetics of GBR830- SAD (Part 1) -Vss^{[9]} |
| title | |
| End point | Secondary |
| type | |
| End point | SAD (Part 1) -Pre-dose, at the end of infusion (+1 minute) |
| timeframe: | and at 1.5, 4, 8, 24 (Day 2), 48 (Day 3), 96 (Day 5), 144 (Day |
| | 7), 168 (Day 8), 336 (Day 15), 672 (Day 29), 1008 (Day 43), |
| | 1344 (Day 57), and 1680 (Day 71) hours after the start of the |
| | infusion. |

^{[9]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 25
Results

| | GBR830, 20 | GBR830, 40 |
|---|---|---|
| | mg/kg dose SAD | mg/kg dose SAD |
| End point values | [Part 1] | [Part 1] |
| Subject group type | Reporting group | Reporting group |
| Number of subjects analysed | 6 | 6 |
| Units: | litre(s) | litre(s) |
| geometric mean (full range | 4.276 (3.34 to 4.97) | 3.892 (3.01 to 5.02) |
| (min-max)) | | |

Secondary endpoints: Pharmacokinetics—Part 2

The parameters monitored during GBR830 SAD (Part 2) pharmacokinetic study include: Cmax at week 1 (see Table 26 for end point information and Table 27 for the results), at week 4 (see Table 28 for end point information and Table 29 for the results) and at week 6 (see Table 30 for end point information and Table 31 for the results); tmax at week 1 (see Table 32 for end point information and Table 33 for the results), at week 4 (see Table 34 for end point information and Table 35 for the results) and at week 6 (see Table 36 for end point information and Table 37 for the results); AUC 0-168 at week 1 (see Table 38 for end point information and Table 39 for the results), at week 4 (see Table 40 for end point information and Table 41 for the results) and at week 6 (see Table 42 for end point information and Table 43 for the results); t1/2 at week 6 (see Table 44 for end point information and Table 45 for the results); CLss at week 4 (see Table 46 for end point information and Table 47 for the results) and at week 6 (see Table 48 for end point information and Table 49 for the results); Vz at week 6 (see Table 50 for end point information and Table 51 for the results); Rac at week 4 (see Table 52 for end point information and Table 53 for the results), and at week 6 (see Table 54 for end point information and Table 55 for the results); Cavg at week 4 (see Table 56 for end point information and Table 57 for the results), and at week 6 (see Table 58 for end point information and Table 59 for the results); C through at week 4 (see Table 60 for end point information and Table 61 for the results), and at week 6 (see Table 62 for end point information and Table 63 for the results).

Secondary: Pharmacokinetics of GBR830—MAD (Part 2)—Week 1—Cmax

TABLE 26
End point information

| | |
|---|---|
| End point | Pharmacokinetics of GBR830- MAD (Part 2)-Week 1- |
| title | Cmax^{[10]} |
| End point | Secondary |
| type | |
| End point | MAD (Part 2) -Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[10]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 27
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 285.4 (249 to 314) | 536.0 (474 to 619) |
| (min-max)) | | |

Secondary: Pharmacokinetics of GBR830—MAD (Part 2)—Week 4—Cmax

TABLE 28
End point information

| | |
|---|---|
| End point | Pharmacokinetics of GBR830- MAD (Part 2)-Week 4- |
| title | Cmax^{[11]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[11]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 29
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 525.7 (482 to 623) | 1060 (965 to 1220) |
| (min-max)) | | |

Secondary: Pharmacokinetics of GBR830—MAD (Part 2)—Week 6—Cmax

TABLE 30
End point information

| | |
|---|---|
| End point | Pharmacokinetics of GBR830- MAD (Part 2)-Week 6- |
| title | Cmax[12] |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

[12]The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 31
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 636.5 (601 to 682) | 1214 (1020 to 1340) |
| (min-max)) | | |

Secondary: Pharmacokinetics of GBR830—MAD (Part 2)—Week 1—tmax

TABLE 32
End point information

| | |
|---|---|
| End point | Pharmacokinetics of GBR830- MAD (Part 2)-Week 1- |
| title | tmax^{[13]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[13]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 33
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | hour | hour |
| median (full range (min-max)) | 1.500 (1.08 to 4.07) | 4.000 (1.53 to 4.02) |

Secondary: Pharmacokinetics of GBR830—MAD (Part 2)—Week 4—tmax

TABLE 34
End point information

| | |
|---|---|
| End point | Pharmacokinetics of GBR830- MAD (Part 2)-Week 4- |
| title | tmax^{[14]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[14]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 35
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: hour | hour | hour |
| median (full range (min-max)) | 4.000 (1.50 to 4.02) | 2.750 (1.00 to 8.00) |

Secondary: Pharmacokinetics of GBR830—MAD (Part 2)—Week 6—tmax

TABLE 36
End point information

| | |
|---|---|
| End point | Pharmacokinetics of GBR830- MAD (Part 2)-Week 6- |
| title | tmax^{[15]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[15]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 37
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | hour | hour |
| median (full range (min-max)) | 1.550 (1.02 to 4.08) | 1.260 (1.00 to 1.52) |

Secondary: Pharmacokinetics of GBR830—MAD (Part 2)—Week 1—AUC 0-168

TABLE 38
End point information

| | |
|---|---|
| End point | Pharmacokinetics of GBR830- MAD (Part 2)-Week 1- |
| title | AUC 0-₁₆₈[16] |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

[16]The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 39
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 25821 (22940 to | 55881 (50310 to |
| (min-max)) | 28480) | 61000) |

Secondary: Pharmacokinetics of GBR830—MAD (Part 2)—Week 4—AUC 0-168

TABLE 40
End point information

| | |
|---|---|
| End point | Pharmacokinetics of GBR830- MAD (Part 2)-Week 4- |
| title | AUC 0-₁₆₈[17] |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

[17]The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 41
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 58690 (56040 to | 133980 (123800 to |
| (min-max)) | 61080) | 145000) |

Secondary: Pharmacokinetics of GBR830—MAD (Part 2)—Week 6—AUC 0-168

TABLE 42
End point information

| | |
|---|---|
| End point | Pharmacokinetics of GBR830- MAD (Part 2)-Week 6- |
| title | AUC 0-168[18] |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

[18]The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 43
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 75439 (69110 to | 157610 (143000 to |
| (min-max)) | 84630) | 171800) |

Secondary: Pharmacokinetics of GBR830—MAD (Part 2)—Week 6—t1/2

TABLE 44
End point information

| | |
|---|---|
| End point | Pharmacokinetics of GBR830- MAD (Part 2)-Week 6- |
| title | t½^{[19]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[19]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 45
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | hour | hour |
| geometric mean (full range | 368.9 (334 to 445) | 432.1 (385 to 544) |
| (min-max)) | | |

Secondary: Pharmacokinetics of GBR830—MAD (Part 2)—Week 4—CLss

TABLE 46
End point information

| | |
|---|---|
| End point | Pharmacokinetics of GBR830- MAD (Part 2)-Week |
| title | 4-CLss^{[20]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[20]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 47
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mL/h | mL/h |
| geometric mean (full range | 13.98 (11.8 to 17.9) | 12.16 (7.94 to 15.9) |
| (min-max)) | | |

Secondary: Pharmacokinetics of GBR830—MAD (Part 2)—Week 6—CLss

TABLE 48
End point information

| | |
|---|---|
| End point | Pharmacokinetics of GBR830- MAD (Part 2)-Week |
| title | 6-CLss^{[21]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[21]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point.

TABLE 49
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: mL/h | mL/h | mL/h |
| geometric mean (full range | 10.94 (9.35 to 12.2) | 10.28 (6.86 to 13.8) |
| (min-max)) | | |

Secondary: Pharmacokinetics of GBR830—MAD (Part 2)—Week 6—Vz

TABLE 50
End point information

| | |
|---|---|
| End point | Pharmacokinetics of GBR830- MAD (Part 2)-Week |
| title | 6-Vss[22] |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

[22]The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point.

TABLE 51
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | litre(s) | litre(s) |
| geometric mean (full range | 5.821 (5.57 to 6.00) | 6.409 (4.20 to 8.85) |
| (min-max)) | | |

Secondary: Pharmacokinetics of GBR830—MAD (Part 2)—Week 4—Rac

TABLE 52
End point information

| | |
|---|---|
| End point | Pharmacokinetics of GBR830- MAD (Part 2)-Week |
| title | 4-Rac^{[23]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[23]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 53
Results

| | GBR830, 10 | GBR830, 20 |
|---|---|---|
| | mg/kg dose MAD | mg/kg dose MAD |
| End point values | [Part | [Part 2] |
| Subject group type | Reporting group | Reporting group |
| Number of subjects analysed | 5 | 6 |
| Units: | mcg · h/mL | mcg · h/mL |
| geometric mean (full range | 2.273 (2.10 to 2.60) | 2.398 (2.20 to 2.60) |
| (min-max)) | | |

Secondary: Pharmacokinetics of GBR830—MAD (Part 2)—Week 6—Rac

TABLE 54
End point information

| | |
|---|---|
| End point | Pharmacokinetics of GBR830- MAD (Part 2)-Week |
| title | 6-Rac^{[24]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[24]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 55
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg · h/mL | mcg · h/mL |
| geometric mean (full range | 2.922 (2.40 to 3.40) | 2.820 (2.70 to 3.00) |
| (min-max)) | | |

Secondary: Pharmacokinetics of GBR830—MAD (Part 2)—Week 4—Cavg

TABLE 56
End point information

| | |
|---|---|
| End point | Pharmacokinetics of GBR830- MAD (Part 2)-Week |
| title | 4-Cavg^{[25]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[25]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 57
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: mcg/mL | | |
| geometric mean (full range | 349.3 (334 to 364) | 797.5 (737 to 863) |
| (min-max)) | | |

Secondary: Pharmacokinetics of GBR830—MAD (Part 2)—Week 6—Cavg

TABLE 58
End point information

| | |
|---|---|
| End point | Pharmacokinetics of GBR830- MAD (Part 2)-Week |
| title | 6-Cavg^{[26]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[26]}The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 59
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 449.0 (411 to 504) | 938.1 (851 to 1023) |
| (min-max)) | | |

Secondary: Pharmacokinetics of GBR830—MAD (Part 2)—Week 4—C trough

TABLE 60
End point information

| | |
|---|---|
| End point | Pharmacokinetics of GBR830- MAD (Part 2)-Week |
| title | 4-C trough ^{[27]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[27]} The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 61
Results

| | GBR830, 10 | GBR830, 20 |
|---|---|---|
| | mg/kg dose MAD | mg/kg dose MAD |
| End point values | [Part 2] | [Part 2] |
| Subject group type | Reporting group | Reporting group |
| Number of subjects analysed | 5 | 6 |
| Units: | mcg/mL | mcg/mL |
| geometric mean (full range | 246.0 (229 to 283) | 638.1 (568 to 748) |
| (min-max)) | | |

Secondary: Pharmacokinetics of GBR830—MAD (Part 2)—Week 6—C trough

TABLE 62
End point information

| | |
|---|---|
| End point | Pharmacokinetics of GBR830- MAD (Part 2)-Week |
| title | 6-C trough ^{[28]} |
| End point | Secondary |
| type | |
| End point | MAD (Part 2)-Pre-dose at first dose, second dose, third dose, |
| timeframe: | fourth dose, fifth dose, sixth dose: at pre- dose, at the end of |
| | infusion (+1 minute), at 1.5, 4, 8, 24, 48, 96, 144, 168, 336, |
| | 672, 1008, and 1344 hours after the start of the infusion. |

^{[28]} The end point is not reporting statistics for all the arms in the baseline period. It is expected all the baseline period arms will be reported on when providing values for an end point on the baseline period. Justification: Statistical analysis was not applicable since there were no inferential statistics, only descriptive statistics were performed for this end point

TABLE 63
Results

| | GBR830, 10 | GBR830, 20 |
|---|---|---|
| | mg/kg dose MAD | mg/kg dose MAD |
| End point values | [Part 2] | [Part |
| Subject group type | Reporting group | Reporting group |
| Number of subjects analysed | 5 | 6 |
| Units: | mcg/mL | mcg/mL |
| geometric mean (full range | 348.0 (307 to 391) | 766.1 (657 to 936) |
| (min-max)) | | |

Secondary Endpoints: Immunogenicity

Anti-drug antibody formation was monitored according to Table 64. The results are reported in Table 65.

Secondary: Anti-Drug Antibodies (ADA) Formation Following GBR830—Immunogenicity

TABLE 64
End point information

| | |
|---|---|
| End point | Anti-drug antibodies (ADA) formation following GBR830 - |
| title | Immunogenicity |
| End point | Secondary |
| type | |
| End point | Blood samples against GBR830 were collected within 15 |
| timeframe: | minutes prior to start of infusion, at Day 15, Day 43, |
| | and Day 71. |

TABLE 65
Results

| | GBR830, | GBR830, | | GBR830, | GBR830, | |
|---|---|---|---|---|---|---|
| | 20 mg/kg | 40 mg/kg | Placebo | 10 mg/kg | 20 mg/kg | Placebo |
| End point | dose SAD | dose SAD | Arm SAD | dose MAD | dose MAD | MAD |
| values | [Part 1] | [Part 1] | [Part 1] | [Part 2] | [Part 2] | [Part 2] |
| Subject group | Reporting | Reporting | Reporting | Reporting | Reporting | Reporting |
| type | group | group | group | group | group | group |
| Number of | 6 | 6 | 4 | 6 | 6 | 4 |
| subjects | | | | | | |
| Units: Number | 0 | 0 | 0 | 0 | 0 | 0 |
| of subjects | | | | | | |

Adverse Events

Adverse event information are summarized in Table 66. As shown in Table 67 no serious adverse event were reported. Non serious adverse event are reported in Table 68 and Table 69.

TABLE 66
Adverse event information
Adverse events information
Timeframe for reporting adverse events:
All adverse events regardless of seriousness,
severity or relationship to the study medications
were recorded from the time of signing the
informed consent form until the follow up contact.

| | |
|---|---|
| Assessment type | Systematic |

Dictionary used

| | |
|---|---|
| Dictionary name | MedDRA |
| Dictionary version | 20.0 |

Reporting group

| | |
|---|---|
| Reporting group title | Part-1, GBR-830, 20 mg/kg [SAD] |
| Reporting group title | Part-1, GBR-830, 40 mg/kg, [SAD] |
| Reporting group title | Part-1, Placebo, [SAD] |
| Reporting group title | Part-2, GBR830, 10 mg/kg [MAD] |
| Reporting group title | Part-2, GBR-830, 20 mg/kg [MAD] |
| Reporting group title | Part-2, Placebo [MAD] |

TABLE 67
Results

| | Part-1, | Part-1, | |
|---|---|---|---|
| Serious adverse events | GBR-830, | GBR-830, | Part-1, |
| Total subjects affected by | 20 mg/kg | 40 mg/kg, | Placebo, |
| serious adverse events | [SAD] | [SAD] | [SAD] |
| subjects affected/exposed | 0/6 (0.00%) | 0/6 (0.00%) | 0/4 (0.00%) |
| number of deaths (all causes) | 0 | 0 | 0 |
| number of deaths resulting | 0 | 0 | 0 |
| from adverse events | | | |
| | Part-2, | Part-2, | |
| Serious adverse events | GBR830, | GBR-830, | Part-2, |
| Total subjects affected by | 10 mg/kg | 20 mg/kg | Placebo |
| serious adverse events | [MAD] | [MAD] | [MAD] |
| subjects affected/exposed | 0/6 (0.00%) | 0/6 (0.00%) | 0/4 (0.00%) |
| number of deaths (all causes) | 0 | 0 | 0 |
| number of deaths resulting | 0 | 0 | 0 |
| from adverse events | | | |

Frequency threshold for reporting non-serious adverse events: 5%

TABLE 68
Results

| | Part-1, | | Part-1, | | | |
|---|---|---|---|---|---|---|
| | GBR-830, | | GBR-830, | | Part-1, | |
| | 20 mg/kg | | 40 mg/kg, | | Placebo, | |
| Non-serious adverse events | [SAD] | | [SAD] | | [SAD] | |
| Total subjects affected by | | | | | | |
| non-serious adverse events | | | | | | |
| subjects affected/exposed | 5/6 | (83.33%) | 5/6 | (83.33%) | 2/4 | (50.00%) |
| Nervous system disorders Headache | | | | | | |
| subjects affected/exposed | 0/6 | (0.00%) | 3/6 | (50.00%) | 1/4 | (25.00%) |
| occurrences (all) | 0 | | 3 | | 1 | |
| General disorders and | | | | | | |
| administration site conditions | | | | | | |
| Catheter site haematoma | | | | | | |
| subjects affected/exposed | 1/6 | (16.67%) | 1/6 | (16.67%) | 0/4 | (0.00%) |
| occurrences (all) | 2 | | 1 | | 0 | |
| Vessel puncture site haematoma | | | | | | |
| subjects affected/exposed | 0/6 | (0.00%) | 2/6 | (33.33%) | 0/4 | (0.00%) |
| occurrences (all) | 0 | | 2 | | 0 | |
| Catheter site swelling subjects | | | | | | |
| affected/exposed | 0/6 | (0.00%) | 0/6 | (0.00%) | 0/4 | (0.00%) |
| occurrences (all) | 0 | | 0 | | 0 | |
| Gastrointestinal disorders | | | | | | |
| Diarrhoea | | | | | | |
| subjects affected/exposed | 2/6 | (33.33%) | 1/6 | (16.67%) | 0/4 | (0.00%) |
| occurrences (all) | 2 | | 1 | | 0 | |
| Flatulence | | | | | | |
| subjects affected/exposed | 2/6 | (33.33%) | 0/6 | (0.00%) | 1/4 | (25.00%) |
| occurrences (all) | 2 | | 0 | | 1 | |
| Abdominal Pain Upper subjects | | | | | | |
| affected/exposed | 1/6 | (16.67%) | 1/6 | (16.67%) | 0/4 | (0.00%) |
| occurrences (all) | 2 | | 1 | | 0 | |
| Infections and infestations | | | | | | |
| Viral upper respiratory tract | | | | | | |
| infection | | | | | | |
| subjects affected/exposed | 1/6 | (16.67%) | 1/6 | (16.67%) | 0/4 | (0.00%) |

| | | | |
|---|---|---|---|
| occurrences (all) | 1 | 1 | 0 |

TABLE 69
Results

| | Part-2, | | Part-2, | | | |
|---|---|---|---|---|---|---|
| | GBR830, | | GBR-830, | | Part-2, | |
| | 10 mg/kg | | 20 mg/kg | | Placebo | |
| Non-serious adverse events | [MAD] | | [MAD] | | [MAD] | |
| Total subjects affected by | | | | | | |
| non-serious adverse events | | | | | | |
| subjects affected/exposed | 5/6 | (83.33%) | 5/6 | (83.33%) | 4/4 | (100.00%) |
| Nervous system disorders | | | | | | |
| Headache | | | | | | |
| subjects affected/exposed | 4/6 | (66.67%) | 1/6 | (16.67%) | 2/4 | (50.00%) |
| occurrences (all) | 4 | | 4 | | 2 | |
| General disorders and | | | | | | |
| administration site conditions | | | | | | |
| Catheter site haematoma subjects | | | | | | |
| affected/exposed | 0/6 | (0.00%) | 0/6 | (0.00%) | 0/4 | (0.00%) |
| occurrences (all) | 0 | | 0 | | 0 | |
| Vessel puncture site haematoma | | | | | | |
| subjects | | | | | | |
| affected/exposed | 2/6 | (33.33%) | 1/6 | (16.67%) | 1/4 | (25.00%) |
| occurrences (all) | 2 | | 2 | | 2 | |
| Catheter site swelling subjects | | | | | | |
| affected/exposed | 1/6 | (16.67%) | 0/6 | (0.00%) | 1/4 | (25.00%) |
| occurrences (all) | 1 | | 0 | | 1 | |
| Gastrointestinal disorders | | | | | | |
| Diarrhoea | | | | | | |
| subjects affected/exposed | 1/6 | (16.67%) | 0/6 | (0.00%) | 1/4 | (25.00%) |
| occurrences (all) | 1 | | 0 | | 1 | |
| Flatulence | | | | | | |
| subjects affected/exposed | 0/6 | (0.00%) | 0/6 | (0.00%) | 0/4 | (0.00%) |
| occurrences (all) | 0 | | 0 | | 0 | |
| Abdominal Pain Upper subjects | | | | | | |
| affected/exposed | 0/6 | (0.00%) | 0/6 | (0.00%) | 0/4 | (0.00%) |
| occurrences (all) | 0 | | 0 | | 0 | |
| Infections and infestations | | | | | | |
| Viral upper respiratory tract | | | | | | |
| infection | | | | | | |
| subjects affected/exposed | 2/6 | (33.33%) | 1/6 | (16.67%) | 1/4 | (25.00%) |

| | | | |
|---|---|---|---|
| occurrences (all) | 3 | 1 | 1 |

### Example 2: An Open-Label, Single-Dose, Parallel-Arm Study to Evaluate the Pharmacokinetics and Absolute Bioavailability of GBR830 Following Single Dose Administration by the Subcutaneous (SC) Route

Study Objectives

The present study was designed to evaluate the absolute bioavailability of GBR830 following SC administration, and the PK of GBR830 following SC and IV administration with the developed formulations to ease administration of GBR830 in further clinical development.

Primary Objective
To determine absolute bioavailability of GBR830 administered by the SC route in healthy adult subjects.

Secondary Objective(s)
To evaluate the PK of GBR830 in healthy adult subjects following single dose SC and IV administration.
To evaluate the immunogenicity after single doses of GBR830 in healthy adult subjects following SC and IV administration.
To evaluate the safety and tolerability of single doses of GBR830 in healthy adult subjects.

Overall Study Design and Plan Description

This study was an open-label, randomized, single-dose, parallel-arm study to evaluate the PK and absolute bioavailability of GBR830 following single dose administration by the SC route. Healthy subjects meeting the eligibility criteria were studied in 3 parallel treatment arms.

Subjects were screened and were admitted to the Clinical Research Unit (CRU) on Day −1 for further eligibility checks. Eligible subjects were randomized to 1 of the 3 treatment arms in a 2:3:3 ratio to receive 600 mg GBR830 IV, 600 mg GBR830 SC, or 75 mg GBR830 SC, respectively. For subjects in the SC arms, GBR830 was administered in the fatty layer of the lower right or left quadrant of the abdomen. Subjects in the IV arm received GBR830 as an IV infusion. Subjects were dosed on the morning of Day 1 and remained at the CRU until the morning of Day 10. Subjects were observed for 10 weeks following administration of the investigational product (IP) to fully characterize the safety and PK profile of GBR830. The maximum study duration from screening to last assessments for each subject was 97 days.

A total of 40 subjects were randomized (10 subjects in the IV arm and 15 subjects in each of the SC arms). There were 8 visits to the study site and the study consisted of 3 phases: screening, treatment and follow-up. The study design is shown in FIG. 2.

After the initial Screening visit (Visit 1), subjects who met all study selection criteria were admitted to the CRU on Day −1 for further eligibility checks. Randomized subjects were dosed in the morning of Day 1 and remained in-house until Day 10 for the 216 hours postdose assessments. On Day 10, the Investigator confirmed the well-being of all subjects prior to their discharge from the CRU. If necessary, subjects remained at the CRU until any adverse events (AEs) causing concern resolved.

Subjects were observed for 10 weeks following administration of the IP to fully characterize the safety and PK profile of GBR830. All subjects returned for outpatient visits and for a final end of study visit on Day 71. The end of the study was defined as the last study visit for the last subject in the study. Any subject experiencing an AE was followed until resolution or stabilization of the AE, or, or return to baseline (if a baseline value was available).

Discussion of the Study Design, Including the Choice of Control Groups

The study was designed as an open-label, randomized, single-dose, parallel-arm study. A parallel-arm design was selected for this study instead of a crossover design because of the 10- to 15-day half-life of the compound.

The study was planned to evaluate the absolute bioavailability of GBR830 following SC administration, and the PK of GBR830 following SC and IV administration. Eligible subjects were randomized to the treatment arms in a ratio of 2:3:3 to receive 600 mg GBR830 IV, 600 mg GBR830 SC and 75 mg GBR830 SC, respectively. Subjects randomized to 1 of the SC groups received an SC injection administered in the fatty layer of the lower right or left quadrant of the abdomen. Subjects in the IV arm received GBR830 by IV infusion. The number of subjects planned in the SC treatment arms was higher than the number of subjects planned in the IV arm, in anticipation that there would be greater variability in PK following SC administration.

Selection of Study Population

Inclusion Criteria

For inclusion into the study, subjects were required to fulfill all of the following criteria:

1. Provision of valid signed and dated written informed consent.

2. Men or women, aged ≥18 years to ≤55 years at the time of informed consent.

3. A body mass index (BMI) of 18.5-32.0 kg/m² (inclusive); weight must have been ≥60 kg.

4. Willing and able to comply with all aspects of the protocol.

5. Otherwise healthy and free from clinically significant illness or disease as determined by medical history, vital signs, physical examinations, electrocardiograms (ECGs), laboratory studies, and other tests performed within 28 days prior to drug administration, as judged by the Investigator.

6. Agreed to the following requirements during the study:
a. Remain in the CRU for up to 10 days.
b. Female subjects of childbearing potential (as judged by the Investigator) who agreed to remain abstinent or use medically acceptable methods of contraception (e.g., implants, injectables, combined oral contraceptives, intrauterine devices [IUDs], double barrier protection) during the study. Male participants who agreed to use a condom with spermicide during intercourse (if not surgically sterilized) during the study.
c. Men should have agreed not to donate sperm for 180 days following administration of the IP.
d. Women must have tested negative for pregnancy at the time of screening and on the day of admission.
e. Women of non-childbearing potential (i.e., were postmenopausal or permanently sterilized [bilateral oophorectomy, hysterectomy, bilateral salpingectomy]) were not required to use contraception. Postmenopausal was defined as at least 1-year post cessation of menses (without an alternative medical cause) with a follicle stimulating hormone (FSH) value of 40.0 IU/L.

Exclusion Criteria

Any of the following was regarded as a criterion for exclusion from the study:
1. A known history of drug or other allergies which in the opinion of the Investigator contraindicated participation.
2. A previous history of hypersensitivity to murine proteins.
3. Had received a live vaccination within 4 weeks prior to randomization, or who intended to receive a live vaccination during the course of the study, or had participated in a vaccine clinical trial within 4 weeks prior to randomization.
4. Had evidence of an active infection as shown by but not limited to fever 38.0° C. (100.4° F.).
5. A documented history of infection(s) within 4 weeks prior randomization.
6. Immunocompromised and/or a documented history of being immunocompromised (congenital or acquired).

7. Had evidence of active or latent tuberculosis as documented in their medical history or test positive for QuantiFERON®¹-TB Gold test.
8. Had a current or a prior history of lymphoproliferative disease or a history of malignant disease; or signs or symptoms suggestive of possible lymphoproliferative disease, including lymphadenopathy or splenomegaly; or active primary or recurrent malignant disease.
9. A history or current evidence of alcohol abuse including a drinking habit of more than 21 units (males) or more than 14 units (females) of alcohol per week or who had a significant history of alcoholism (1 unit of alcohol equals half pint [285 mL] of beer or lager, 1 glass [125 mL] of wine, or ⅙ gill [25 mL] of spirits).
10. A history of drug abuse which in the opinion of the Investigator was considered to be clinically significant or who tested positive for drugs of abuse at screening or admission.
11. Unwilling to abstain from methylxanthine-containing beverages or food (coffee, tea, cola, chocolate, “power drinks”) for 48 hours prior to admission to the CRU and during their in-house stay.
12. Positive test results for disease markers of human immunodeficiency virus (HIV), Hepatitis B or Hepatitis C.
13. An abnormal ECG (including a QTc >450 msec) which was considered clinically significant in the opinion of the Investigator at screening (QTc calculated based on the Fridericia correction formula).
14. Laboratory test results with values that were significantly outside the normal reference ranges and/or judged to be clinically significant by the Investigator, including but not limited to:
   Serum creatinine >1.5 mg/dL (equivalent to Systeme International [SI] unit of >114.39 μmol/L), blood urea nitrogen (BUN) upper limit of normal (ULN) at screening.
   Alanine amino transferase (ALT) or aspartate amino transferase (AST) times ULN, and/or serum total bilirubin ≥1.5 times ULN, at screening.
   Hemoglobin (Hb) value less than 11 g/dL (equivalent to SI unit of less than 6.83 mmol/L) at screening.
   Absolute neutrophil count ≤500/μL (equivalent to SI unit of ≤1.5*10⁹/L) or absolute lymphocyte count ≤800/μL (equivalent to SI unit of ≤0.8*10⁹/L) or platelet count ≤100000/μL (equivalent to SI unit of ≤100*10⁹/L) or any abnormal evaluations judged clinically significant by the Investigator at screening and at predose.
15. Any evidence of organ dysfunction and/or any clinically meaningful deviation from the normal in history, physical determinations or investigations or subjects with a clinical condition or who are receiving therapy that, in the opinion of the Investigator, would have made them unsuitable for study.
16. A history of current or previous psychiatric illnesses or previous psychiatric events judged clinically significant by the Investigator.
17. Had participated in a study with a new molecular entity or any other drug trial within 3 months of IP administration or had previously received GBR830.
18. Previous exposure to antibody therapies or administration of immunoglobulins (Ig) within 6 months of randomization.
19. A history of donating 1 unit of blood (450 mL) in the 3 months prior to IP administration or who intended to donate within 3 months of their last scheduled study visit.
20. A history of or presence of autoimmune conditions.
21. A supine blood pressure >150/90 mmHg at screening or a history of recurrent hypertensive or hypotensive events considered as clinically relevant or documented orthostatic hypotension or a supine blood pressure <90/45 mmHg at screening.
22. A history of recurrent/severe/undiagnosed chest pain or a history of cardiovascular abnormalities of any New York Heart Association criterion, or arrhythmia or stable or unstable angina or who have any clinical or radiological evidence of cardiovascular disease.
23. A recent history of clinically meaningful illnesses including but not limited to, gastrointestinal, hepatic, renal, cardiovascular, respiratory, neurological, hematological disease, metabolic endocrine and immunological disorders.
24. Currently taking or who had taken any prescription or non-prescription medication within 7 days or 5 half-lives (if known) (whichever is longer) of dosing, including aspirin, vitamins, herbal and dietary supplements (except paracetamol and/or hormone replacement therapies).

Removal of Subjects from Therapy or Assessment

Subjects were removed from the study if any of the following circumstances occurred:
Withdrawal of consent by the subject to continue in the study.
Development of a serious or intolerable AE, which at the discretion of the Investigator, necessitated discontinuation from the study.
The Investigator believed continued participation was not in the best interest of the subject.
The Investigator believed the subject had not adhered to the study procedures or restrictions.
A positive pregnancy test.
A protocol deviation/violation, which in the opinion of the Sponsor and Investigator, warranted discontinuation from the study.
A subject required concomitant medications, which may have interfered with the PK of the IP. Note: withdrawal in such cases was to be discussed and mutually agreed by the Investigator and the Sponsor; however, in this study, no such events occurred.

Treatments

Treatments Administered

GBR830 was provided as lyophilized powder in 10 mL glass vial and was reconstituted with sterile water for injection. The following treatments were administered in the study:
Treatment A: a single dose of 600 mg GBR830 as an IV infusion for 60 minutes.
Treatment B: a single dose of 600 mg GBR830, as 2 SC injections of 2 mL, each at 2 separate sites in the fatty layer of the lower right or left quadrant of the abdomen. Each mL of study drug solution contained 150 mg of GBR830.
Treatment C: a single dose of 75 mg GBR830 as 1 SC injection of 0.5 mL in the fatty layer of the lower right or left quadrant of the abdomen. The 0.5 mL solution of study drug contained 75 mg of GBR830.

Method of Assigning Subjects to Treatment Groups

A total of 40 subjects were randomized to 1 of the 3 treatment arms in a 2:3:3 ratio on Day 1 (Visit 2). Subjects were randomly assigned to treatments based on a computer-generated randomization scheme that was reviewed and approved by an independent statistician.

Selection of Doses in the Study

Dose levels of 75 mg GBR830 and 600 mg GBR830 were selected in this study because these levels encompass the anticipated efficacy dose range based on preclinical experiments. The selected doses were either equivalent to or lower than the dose levels demonstrated to be safe in the previous phase 1 clinical study. The safety, tolerability and PK of GBR830 were evaluated in healthy adult subjects after single IV administration over a dose range of 0.3 to 10 mg/kg. GBR830 was found to be safe and well-tolerated across the tested dose range. In vitro pharmacodynamics studies showed that GBR830 was devoid of agonistic potential and did not induce cytokine release in either human peripheral whole blood from healthy subjects or in human peripheral blood mononuclear cell cultures at high density. Taken together, the results of these studies suggested that GBR830 has a very low risk of inadvertent cytokine release in humans.

Selection and Timing of Dose for Each Subject

The dose and route of administration for each subject was randomly assigned based on the randomization scheme.

Pharmacokinetics

Pharmacokinetics Parameters

Blood samples (3.5 mL each) were collected for assessment of serum concentrations of GBR830 and PK parameters for GBR830, as per routine phlebotomy procedures.

The PK parameters in Table 70 were estimated for GBR830 in this study.

TABLE 70
Definition of Pharmacokinetic Parameters

| Parameter | Description |
|---|---|
| Cₘₐₓ | Maximum serum concentration. |
| Cₘₐₓ/Dn | Dose normalized Cₘₐₓ, where Dn was calculated as Dose/Weight (kg) |
| tₘₐₓ | Time to attain maximum serum concentration. First observed time to reach peak |
| | analyte concentration obtained directly from the experimental data without |
| | interpolation, expressed in hours. |
| AUC₍₀₋ₗₐₛₜ₎ | Area under the concentration-time curve (time 0 to time of last quantifiable |
| | concentration). The AUC₀₋ₗₐₛₜ was calculated using the ‘linear up-log down’ |
| AUC₍₀₋ₗₐₛₜ₎/Dn | Dose normalized AUCₗₐₛₜ where Dn was calculated as dose/weight (kg) |
| AUC_{(0-∞)} | Area under the serum concentration-time curve (time 0 to infinity) |
| AUC_{(0-∞)}/Dn | Dose normalized AUC_{(0-∞)} where Dn was calculated as dose/weight (kg) |
| AUC₍₀₋₁₆₈₎ | Area under the concentration-time curve (time 0 to time 168 hours). The |
| | AUC₀₋₁₆₈ was calculated using the ‘linear up-log down” trapezoidal rule |
| % AUC | Percentage of AUC(0-∞) that is due to extrapolation from the time of last |
| extra | observed concentration to infinity |
| λ_{z} | Apparent termination phase rate constant calculated by linear regression of the |
| | terminal log- linear portion of the concentration vs. time curve. Linear regression |
| | of at least three points and an adjusted r2 greater than 0.80 are required to retain |
| | λ_{z} and associated parameters. |
| t½ | Apparent terminal elimination half-life |
| CL(/F) | Total clearance |
| V_{z}(/F) | Apparent volume of distribution associated with the terminal phase |
| Vₛₛ | Volume of distribution at steady state |

Immunogenicity Assessments

Blood samples (5 mL each) were collected to detect anti-drug antibodies (ADAs) to GBR830. The procedure for the collection of blood samples for ADA assessment was similar to the procedure used for the collection of blood for PK samples.

Drug Concentration Measurements

Serum concentrations of GBR830 were quantified using a validated enzyme-linked immunosorbent assay (ELISA) method.

The GBR 830 enzyme immunoassay is a direct enzyme immunoassay. The antibody GBR 830 (IgG) in calibration standard samples, quality control samples and validation samples was captured by the antigen hOX40-His coated on a 96-well plate. Fcγ fragment specific peroxidase-conjugated Affinipure goat anti-human IgG antibody (e.g. AB_2337577) binds to GBR 830 immobilised on the plate by antigen. Upon addition of tetramethylbenzidine (TMB) substrate a blue colour was developed. Sulphuric acid was added to stop the reaction. The absorbance is measured at 450 nm (Ref. 620 nm).

Characteristics of the validated method are given below:
Calibrated Range: 0.977-62.5 ng/mL
Minimum Required Dilution (MRD): 1: 100
Defined LLOQ*: 97.7 ng/mL (0.977 ng/mL×100)
Defined ULOQ*: 6250 ng/mL (62.5 ng/mL×100)
Intra-assay Accuracy [bias %]: Between −15.3% and 3.21% and **between −13.0 and 1.15%
Intra-assay Precision [CV %]: (ICH: Repeatability) Between 1.88% and 5.74% and **between 2.59 and 9.21%
Inter-assay Accuracy [bias %]: Between −3.61% and 2.61% and **between −11.2 and 0.209%
Inter-assay Precision [CV %]: (ICH: Intermediate Precision) Between 5.50% and 13.1% and **between 3.84 and 10.8%
Total Error [TE %]: (Overall, inter- and intra-assay) Between 3.37% and 17.2% and **between 2.87 and 22.0%

*: Due to 100 fold dilution of validation samples the defined LLOQ and ULOQ values are 100 times higher than the measured concentration

All individual serum GBR830 concentration data are listed including the actual PK sampling time since dosing along with the sampling time deviations in minutes or hours. GBR830 serum concentration data are summarized by descriptive statistics for the pharmacokinetic analysis set (PKAS). Serum GBR830 concentrations below the quantifiable limit (BQL) were set to one-half the lower limit of quantification (LLOQ) in the computation of descriptive statistics. If over one-half the subjects at a given time for a given treatment have values BQL then only the minimum and maximum were displayed.

Statistical Methods Planned in the Protocol and Determination of Sample Size

Statistical and Analytical Plans

All data were summarized with descriptive statistics (number of observations, arithmetic mean, standard deviation [SD], minimum [min], median and maximum [max]) for continuous variables, and frequency and percentage for categorical data. Coefficient of variation (CV) of the arithmetic mean, geometric mean and CV of the geometric mean (geoCV) were provided for PK concentrations and parameters. All analyses were performed using SAS Software version 9.4. Datasets were prepared using headings from Clinical Data Interchange Standard Consortium (CDISC) Analysis Data Model (ADaM) version 2.1. Pharmacokinetic parameter calculations were primarily done using Phoenix WinNonlin version 6.3 (Pharsight, Inc). Additional PK computations may have been performed in SAS.

Study Endpoints

The primary endpoint was systemic exposure of GBR830 characterized by Cmax and area under the serum concentration-time curve (AUC) from time zero to the last sampling time at which the drug concentration was at or above the LLOQ (AUC0-last) and AUC from time zero to infinity (AUC0-∞).

Secondary endpoints were as follows:
Serum PK parameters of GBR830: AUC from time 0 to 168 hours postdose (AUC₀₋₁₆₈), time corresponding to Cmax (tmax), last sampling time at which the drug concentration was at or above the lower limit of quantification, clearance (CL or clearance after extravascular administration [CL/F]), apparent volume of distribution (Vz or Vz/F), volume of distribution at steady state (Vss), apparent terminal elimination rate constant (λz) and terminal elimination half-life (t½).
Anti-drug antibody (ADA) formation.
Treatment-emergent adverse events (TEAEs) and SAEs.

Analysis of the Primary Endpoint

The primary endpoint was the systemic exposure of GBR830 characterized by Cmax and AUC0-last and AUC0-∞. Pharmacokinetic parameters for GBR830 were estimated using non-compartmental methods with Phoenix WinNonlin. The absolute bioavailability of GBR830 was calculated as described in the SAP as the dose-corrected AUC of the SC administration divided by the dose-corrected AUC of the IV administration. The dose correction was done in 2 ways, dividing by dose and dividing by dose and multiplying by body weight, to see the effect of including body weight. An analysis of variance (ANOVA) was performed on AUC0-last, AUC0-∞ and Cmax using PROC MIXED in SAS.

Analysis of the Secondary Endpoints

A secondary endpoint, the serum PK parameters of GBR830, were estimated from the concentration-time profiles for all subjects in the PKAS population. In estimating the PK parameters, BQL values at the beginning of the profile were set to zero. Any BQL values that occurred after the first quantifiable point were considered missing. Actual sampling times, rather than scheduled sampling times, were used in all computations involving sampling times. Another secondary endpoint, ADA formation, was evaluated using the results of the immunogenicity bioanalysis. If the subject had a postdose confirmed positive result, the subject was flagged as positive. Otherwise, the subject was flagged as negative. The impact of ADA on individual subject PK parameters (AUC0-last, AUC0-∞, Cmax, and CL/F) and geometric mean were plotted versus treatment, with separate symbols for ADA negative and ADA positive subjects.

Analysis of Safety Endpoints

Adverse Events

The nature, incidence, intensity, as well as the causality assessment were reported for each AE. All AEs were coded according to MedDRA version 20.0. All AEs (including non-treatment-emergent events) recorded on the eCRF were listed. Any new AEs or worsening of an existing condition, occurring after administration of the study drug up to and including the follow-up visit were defined as TEAEs and were listed and summarized by MedDRA System Organ Class (SOC) and Preferred Term (PT), according to the randomized treatment received. Summaries of the number of subjects with TEAEs and the number of TEAEs experienced were presented by treatment, relationship, and severity. A breakdown of the number of events and the number and percentage of subjects reporting each TEAE, SAEs, TEAEs leading to discontinuation, and TEAEs related to study drug categorized by SOC and PT were summarized or listed by treatment.

Laboratory Data

Descriptive statistics (number of subjects, mean, SD, median, min and max) were presented for laboratory data (hematology, coagulation, chemistry, and urinalysis) including change from baseline by treatment, visit day, and scheduled time. All laboratory data, including change from baseline were listed, accompanied by an indication if the parameter was outside the reference range. In addition, a separate summary listing of all data outside the laboratory reference range was provided. Shift tables presented changes from baseline in laboratory data (categorized as normal, abnormal, not clinically significant, and abnormal clinically significant) to worst postdose value with the number and percentage (%) relative to normal ranges. All laboratory data were listed by treatment and subject and presented using SI units (also used in the Study Data Tabulation Model [SD™] Controlled Terminology).

Vital Signs

Descriptive statistics (number of subjects, mean, SD, median, minimum and maximum) were used to summarize vital sign results and changes from baseline by treatment, visit day, and scheduled time. All vital signs data (SBP, DBP, pulse rate, supine respiratory rate, and body temperature) were listed including the change from baseline.

Electrocardiograms

All ECG data (including change from baseline and results of the evaluation by the Investigator) were listed. Triplicate ECG data were taken if there were any abnormalities. Descriptive statistics (number of subjects, mean, SD, median, min, and max) for ECG parameters and changes from baseline were presented by treatment, visit day, and scheduled time. Triplicate ECGs, if any, were averaged. If the last predose ECGs were performed in triplicate, the mean was used as the baseline. In addition, shift tables presented changes from baseline in ECG interpretation (categorized as normal; abnormal, not clinically significant; and abnormal, clinically significant) to end of treatment (or end of phase or by visit). In case of available triplicate ECGs, the worst (most abnormal) evaluation was used in the shift tables.

Determination of Sample Size

Assessment of the absolute bioavailability of GBR830 after SC administration was the objective of the study. Thus, no formal statistical sample size estimation was conducted and the sample size was chosen based on practical consideration. Forty subjects were randomized to 3 treatment arms, with 10 subjects in the IV arm and 15 subjects in each of the SC arms.

A sample size of 10 subjects in the IV arm was considered appropriate based on the PK data from a previous single dose IV study in 10 healthy subjects. As there was a potential for higher inter-individual variability in PK with GBR830 administered SC compared to GBR830 administered IV, a higher sample size (15 subjects) for each SC arm was considered appropriate.

Study Subjects

Disposition of Subjects

A summary of subject disposition is presented in Table 71 and FIG. 3. A total of 40 subjects were randomized to study treatment. All 40 subjects were included in the SAF, and 39 subjects were included in the PKAS. One subject (subject number 34) in the 600 mg GBR830 IV treatment group was excluded from the PKAS due to premature discontinuation of infusion due to an AE, leading to administration of less than the planned dose level. The majority of the subjects completed the study (n=38, 95%). There were 2 subjects in the 75 mg GBR830 SC treatment group that did not complete the study; 1 subject (subject number 20) withdrew consent on Day 43 after sampling and 1 subject (subject number 26) was lost to follow-up (missed visits on Days 29, 43, and 71).

TABLE 71
Summary of Subject Disposition.

| | 600 mg | 600 mg | 75 mg | | |
|---|---|---|---|---|---|
| | GBR830 IV | GBR830 SC | GBR830 SC | | Total |
| | (N = 10) | (N = 15) | (N = 15) | | (N = 40) |
| | n (%) | n (%) | n (%) | | n (%) |
| Randomized | 10 (100) | 15 (100) | 15 | (100) | 40 (100) |
| Dosed | 10 (100) | 15 (100) | 15 | (100) | 40 (100) |
| PK Analysis Set | 9 (90) | 15 (100) | 15 | (100) | 39 (98) |
| Completed | 10 (100) | 15 (100) | 13 | (87) | 38 (95) |
| Withdrew | | | 2 | (13) | 2 (5) |
| Lost to follow-up | | | 1 | (7) | 1 (3) |
| Withdrawal by | | | 1 | (7) | 1 (3) |

IV = intravenous; SC = subcutaneous; n = number of subjects; N = number of subjects per treatment in this analysis set; PK = pharmacokinetic.

Pharmacokinetics and Immunogenicity Evaluation Data Sets Analyzed

For this study, 2 analysis sets were included, the SAF and the PKAS.
The SAF consisted of all subjects who received study medication and was used for safety analyses.
All 40 subjects were included in the SAF.
The PKAS consisted of the subset of the SAF population for which sufficient serum concentration data were available to facilitate derivation of at least 1 PK parameter and for whom the time of dosing on the day of sampling was known.
In the PKAS, One subject (Subject 34; 600 mg GBR830 IV) was excluded because of premature discontinuation of the study drug infusion; this resulted in a final analysis set of 39 subjects (Table 71). This protocol deviation may have affected the PK assessment; therefore the subject was excluded from PK analysis.
Subject 34 received only 242 mL of the 273-mL infusion volume, which was less than 90% of the planned dose, due to development of urticaria; therefore, data from this subject were excluded from the PKAS.

Demographic and Other Baseline Characteristics

As shown Table 72 to Table 74 for the SAF, the mean age of study subjects was 29 to 31 years across treatment groups. Subjects ranged in age from 19 to 54 years. The distribution of male and female subjects was approximately equal in all groups and the total study population distribution (22 males, 55% and 18 females, 45%), with the exception of the 600 mg GBR830 IV group, in which there were more male subjects (7 subjects, 70%) than female subjects (3 subjects, 30%). The majority of the subjects were white. There were no important differences across treatment groups.

TABLE 72
Summary of Demographic Data (Safety Analysis Set)
Height, weight and BMI determined at screening.

| Parameter | Treatment | N | Mean | | Median | Min, Max |
|---|---|---|---|---|---|---|
| Age (y) | 600 mg GBR830 IV | 10 | 29 | (10) | 25 | 19, 54 |
| | 600 mg GBR830 | 15 | 30 | (10) | 26 | 20, 49 |
| | 75 mg GBR830 SC | 15 | 31 | (13) | 24 | 20, 54 |
| | Total | 40 | 30 | (11) | 25 | 19, 54 |
| Weight | 600 mg GBR830 IV | 10 | 76.5 | (8.1) | 76.1 | 66.5, |
| (kg) | 600 mg GBR830 | 15 | 79.6 | | 76.4 | 65.0, |
| | 75 mg GBR830 SC | 15 | 76.7 | | 76.0 | 60.9, |
| | Total | 40 | 77.7 | | 76.2 | 60.9, |
| BMI | 600 mg GBR830 IV | 10 | 24.5 | (1.9) | 24.1 | 21.8, |
| (kg/m²) | 600 mg GBR830 | 15 | 25.3 | (3.6) | 24.0 | 20.1, |
| | 75 mg GBR830 SC | 15 | 24.0 | (3.2) | 24.5 | 19.2, |
| | Total | 40 | 24.6 | (3.1) | 24.2 | 19.2, |

BMI = body mass index; IV = intravenous; max = maximum; min = minimum; N = number of subjects per treatment in this analysis set; n = number of subjects; SC = subcutaneous; SD = standard deviation; yr = year.

TABLE 73
Summary of Demographic Data (Safety Analysis Set)
Height, weight and BMI determined at screening.

| | | |
|---|---|---|
| Parameter | Treatment | n (%) |

Gender (N = 40)

| | Male | 600 mg GBR830 | 7 | (70%) |
|---|---|---|---|---|
| | | 600 mg GBR830 | 8 | (53%) |
| | | 75 mg GBR830 SC | 7 | (47%) |
| | | Total | 22 | (55%) |
| | Female | 600 mg GBR830 | 3 | (30%) |
| | | 600 mg GBR830 | 7 | (47%) |
| | | 75 mg GBR830 SC | 8 | (53%) |
| | | Total | 18 | (45%) |
| Race (N = 40) | | | | |
| | White | 600 mg GBR830 | 10 | |
| | | 600 mg GBR830 | 13 | (87%) |
| | | 75 mg GBR830 SC | 13 | (87%) |
| | | Total | 36 | (90%) |
| | American Indian or | 600 mg GBR830 | 0 | |
| | Alaska Native | 600 mg GBR830 | 1 | (7%) |
| | | 75 mg GBR830 SC | 0 | |
| | | Total | 1 | (3%) |
| | Black or African | 600 mg GBR830 | 0 | |
| | American | 600 mg GBR830 | 1 | (7%) |
| | | 75 mg GBR830 SC | 0 | |
| | | Total | 1 | (3%) |
| | White + Black or | 600 mg GBR830 | 0 | |
| | African American | 600 mg GBR830 | 0 | |
| | | 75 mg GBR830 SC | 1 | (7%) |
| | | Total | 1 | (3%) |
| | White/Black/ | 600 mg GBR830 | 0 | |
| | American | 600 mg GBR830 | 0 | |
| | Indian | 75 mg GBR830 SC | 1 | (7%) |
| | | Total | 1 | (3%) |

BMI = body mass index; IV = intravenous; max = maximum; min = minimum; N = number of subjects per treatment in this analysis set; n = number of subjects; SC = subcutaneous; SD = standard deviation; yr = year.

TABLE 74
Summary of Demographic Data (Safety Analysis Set)
Height, weight and BMI determined at screening.

| Parameter | Treatment | n (%) | |
|---|---|---|---|
| Hispanic or | 600 mg GBR830 | 1 | (10%) |
| Latino | 600 mg GBR830 | 0 | |
| | 75 mg GBR830 SC | 0 | |
| | Total | 1 | (3%) |
| Not Hispanic | 600 mg GBR830 | 9 | (90%) |
| or Latino | 600 mg GBR830 | 15 | (100%) |
| | 75 mg GBR830 SC | 15 | (100%) |
| | Total | 39 | (98%) |

BMI = body mass index; IV = intravenous; max = maximum; min = minimum; N = number of subjects per treatment in this analysis set; n = number of subjects; SC = subcutaneous; SD = standard deviation; yr = year.

Measurements of Treatment Compliance

The infusion was stopped for 2 subjects receiving GBR830 600 mg IV.
Subject 32 received the full infusion volume of 273 mL; however, the infusion was stopped at 09:39 for a duration of 5 seconds, and again stopped at 10:05 for a duration of 3 seconds.
Subject 34 received 242 mL of the 273-mL infusion volume. The infusion was discontinued early due to development of urticaria and this subject was excluded from the PKAS.

Pharmacokinetics Results and Tabulations of Individual Subject Data

Serum Concentration Profile of GBR830

GBR830 at 600 mg IV:

Following a single 1.0-hour IV infusion of 600 mg GBR830, maximum serum concentrations (Cmax) were reached at or after the end of infusion, i.e., between 1.00 hour to 6.00 hours from the start of infusion. After the Cmax, the serum concentration profiles generally declined in bi-phasic manner with a short distribution phase of about 24 to 48 hours, followed by a relatively longer terminal elimination phase extending up to the last sampling point (1680 hours). Following 600 mg IV, the serum concentrations were quantifiable (>LLOQ=97.7 ng/mL) up to the last sampling time point (1680 hours) in all subjects.

GBR830 at 600 mg SC:

Following SC injection of 600 mg GBR830, the serum concentrations increased gradually with time and the Cmax was reached at 48 to 192 hours, post dosing. The median tmax was 120 hours (5 days), post dosing. After Cmax, the serum concentrations generally declined in a mono-phasic manner extending up to the last sampling point. Following 600 mg GBR830 SC injection, the serum concentrations were quantifiable (>LLOQ=97.7 ng/mL) up to the last sampling time point (1680 hours) in all subjects. Comparison of the geometric mean serum concentration profiles of GBR830 after SC and IV at 600 mg suggested that the elimination phase after SC and IV are parallel to each other.

GBR830 at 75 mg SC:

Following SC injection of 75 mg GBR830, the serum concentrations increased gradually with time and the Cmax was reached at 48 to 192 hours, post dosing. The median tmax was 96 hours (4 days), post dosing. After Cmax, the serum concentrations generally declined in a mono-phasic manner up to the last time point where GBR830 was quantifiable. Following 75 mg GBR830 SC injection, serum concentrations were quantifiable (>97.7 ng/mL) in all subjects up to 1008 hours postdose, where samples were available for bioanalysis. At the last sampling point (1680 hours), serum samples were available from all subjects (2 subjects were excluded as described below) for bioanalysis, and 6 of those had quantifiable levels at this time point.

One subject (Subject 20), who received 75 mg GBR830 SC, withdrew consent on Day 43 of the study. The PK samples were collected up to Day 43 (1008 hours) from this subject and showed quantifiable serum concentrations of GBR830 until Day 43. One subject (Subject 26), who received 75 mg GBR830 SC, missed 3 PK samples (Days 29, 43 and 71). Although the PK sample on Day 57 was collected, the serum concentrations were below quantifiable limit for this subject. Therefore, for this subject quantifiable serum concentrations up to Day 15 were used for calculation of PK parameters.

Overall, the inter-subject variability, as evaluated by geo CV, in serum concentration profile was higher after SC injection in comparison to that of IV infusion (e.g., at Cmax after 600 mg GBR830 SC: 32.3% vs 17.5% at Cmax after 600 mg IV) (FIG. 4 and FIG. 5).

Pharmacokinetics Parameters

The following analyzed pharmacokinetic parameters are described below in detail and reported in Table 75: tmax, Cmax, AUC, Vss, CL, ty and between-subject variability.

Tmax:

After single IV infusion, the median tmax was 2.00 hours from the start of the infusion (range: 1.00 to 6.00 hours). After SC injection of 600 mg GBR830 and 75 mg GBR830, the median tmax was 120 hours (5 days) and 96 hours (4 days), respectively, with individual tmax ranging from 48 hours to 192 hours.

Cmax and AUC:

The geometric mean Cmax after 600 mg IV infusion was 191 μg/mL. The geometric mean Cmax after 600 mg GBR830 SC injection and 75 mg GBR830 SC injection was 59.7 μg/mL and 8.59 μg/mL, respectively. The geometric mean AUC0-∞ after 600 mg GBR830 SC injection and 75 mg GBR830 SC injection were 39127 μg*h/mL and 4668 μg*h/mL, respectively. With an 8-fold increase in dose level, from 75 mg GBR830 SC to 600 mg GBR830 SC, the Cmax and AUC0-∞ increased approximately 7-fold and 8.4-fold, respectively; this indicated a dose proportional increase in PK over the range of 75 mg to 600 mg administered SC.

Volume of Distribution (Vss and Vz):

The geometric mean Vss after IV infusion was 5.03 L, indicating limited volume of distribution. The geometric mean volume of distribution based on the terminal phase (Vz) after IV infusion was 5.17 L, also indicating limited distribution. The geometric mean volume of distribution based on the terminal phase (Vz/F) after SC injection ranged from 6.23 L to 8.05 L.

Clearance and Half-life:

The geometric mean CL after IV infusion was estimated to be 10.2 mL/h (0.0102 L/h), suggesting slow clearance. The geometric mean clearance (CL/F) after SC injection ranged from 0.0153 to 0.0161 L/h. Consistent with the slow clearance GBR830 showed long terminal elimination half-life across the dose levels. The geometric mean terminal elimination half-lives (t½) after 600 mg IV infusion, 600 mg GBR830 SC injection and 75 mg GBR830 SC injection were 353 hours, 364 hours and 269 hours, respectively. The t½ after 75 mg was slightly shorter than that at the 600 mg dose level, however there was considerable overlap in individual t½ between the dose levels.

Between-Subject Variability:

The between-subject variability, as evaluated by geoCV, in PK parameters was generally lower with IV infusion (% CV for AUC0-∞ 600 mg IV=16.2%) in comparison to that after SC injection (% CV for AUC0-∞ 600 mg GBR830 SC=32.4%). The between-subject variability at 75 mg GBR830 SC (% CV for AUC0-∞=48.4%) was slightly higher than the between-subject variability at 600 mg GBR830 SC.

TABLE 75
Summary of Pharmacokinetic Parameters (PKAS).

| | | 600 mg | | 600 mg | | 75 mg | |
|---|---|---|---|---|---|---|---|
| | | GBR830 IV | | GBR830 SC | | GBR830 SC | |
| Parameter | Statistic | (N = 9) | | (N = 15) | | (N = 15) | |
| Cₘₐₓ (μg/mL) | Geometric | 191 | (17.5) | 59.7 | (32.3) | 8.59 | (47.1) |
| | mean (geoCV) | | | | | | |
| | Min-Max | 145-238 | | 31.4-88.6 | | 2.93-17.2 | |
| tₘₐₓ (h) | Median | 2.00 | | 120 | | 96 | |
| | Min-Max | 1.00-6.00 | | 48-192 | | 48-192 | |
| AUC₍₀₋₁₆₈₎ | Geometric | 17568 | (10.7) | 7389 | (36.2) | 1061 | (46.2) |
| (μg*h/mL) | mean (geoCV) | | | | | | |
| | Min-Max | 14611-20926 | | 3809-12740 | | 380-2310 | |
| AUC₍₀₋ₗₐₛₜ₎ | Geometric | 56894 | (14.1) | 37188 | (31.8) | 4092 | (53.4) |
| (μg*h/mL) | mean (geoCV) | | | | | | |
| | Min-Max | 46198-72347 | | 19041-54611 | | 1588-8727 | |
| AUC_{0-∞} | Geometric | 59105 | (16.2) (d) | 39127 | (32.4) | 4668 | (48.4) (f) |
| (μg*h/mL) | mean (geoCV) | | | | | | |
| | Min-Max | 48281-77329 | | 19687-57056 | | 1682-8794 | |
| CL (mL/h) or | Geometric | 10.2 | (16.2) (d) | 15.3 | (32.4) | 16.1 | (48.4) (f) |
| CL/F (mL/h)^{a} | mean (geoCV) | | | | | | |
| | Min-Max | 7.76-12.4 | | 10.5-30.5 | | 8.53-44.6 | |
| V_{z} (L) | Geometric | 5.17 | (15.7) (d) | 8.05 | (31.4) | 6.23 | (49.2) (f) |
| V_{z}/F (L)^{a} | mean (geoCV) | | | | | | |
| | Min-Max | 4.40-6.79 | | 5.36-14.2 | | 2.75-19.7 | |
| Vₛₛ (L) | Geometric | 5.03 | (14.6) (d) | NA | | NA | |
| | mean (geoCV) | | | | | | |
| | Min-Max | 4.40-6.51 | | NA | | NA | |
| t½ (h) | Geometric | 353 | (15.2) (d) | 364 | (13.6) | 269 | (24.8) (f) |
| | mean (geoCV) | | | | | | |

| | | | |
|---|---|---|---|
| Min-Max | 298-429 | 284-435 | 160-351 |

Numbers of non-missing values are different from the treatment N: (d): n = 8; (f): n = 13.
AUC₀₋₁₆₈ = AUC from time 0 to 168 hours postdose; AUC₀₋ₗₐₛₜ = AUC from time 0 to the last sampling time at which the drug concentration was at or above the lower limit of quantification; AUC_{0-∞} = AUC from time 0 to infinity; Cₘₐₓ = maximum serum concentration; CL = clearance; CL/F = clearance after non-intravenous administration; geoCV = geometric coefficient of variation; IV = intravenous; max = maximum; min = minimum; NA = not applicable; SC = subcutaneous; t½ = terminal elimination half-life; tₘₐₓ = time corresponding to Cₘₐₓ; Vₛₛ = volume of distribution at steady state; V_{z} = apparent volume of distribution based on the terminal phase; V_{z}/F = apparent volume of distribution based on the terminal phase after non-intravenous administration.
^{a}CL and V_{z} for GBR830 600 mg IV; CL/F and V_{z}/F for GBR830 600 mg and GBR830 75 mg SC

Estimation of Absolute Bioavailability

The absolute bioavailability after SC injection was determined by comparing the dose corrected AUC_{0-∞} at 75 mg and 600 mg to that after IV infusion at 600 mg. The absolute bioavailability after 600 mg GBR830 SC and 75 mg GBR830 SC were estimated to be 0.662 (66.2%) and 0.632 (63.2%), respectively. In order to adjust for the body weight differences in dose groups, the dose and body weight normalized AUC_{0-∞} after SC injection was also compared with that after IV infusion at 600 mg. The absolute bioavailability estimates when adjusted for body weight were 0.693 (69.3%) at 600 mg GBR830 SC and 0.641 (64.1%) at 75 mg (Table 76).

TABLE 76
Summary of Bioavailability. Using PROC MIXED
(ANOVA) with treatment as a fixed effect.

| | | Geometric LS | | Test/Reference Ratio | | | Between- |
|---|---|---|---|---|---|---|---|
| | Parameter | means (μg*h/mL) | | | 90% CI | | Subject |
| Dose Level | Evaluated | Test | Reference | Estimate | Lower | Upper | [symbol] |

Analysis by actual dose

| | | | | | | | |
|---|---|---|---|---|---|---|---|
| 600 mg GBR830 | AU | 65.2 | 98.5 | 0.662 | 0.51 | 0.86 | 36. |
| 75 mg GBR830 SC | AU | 62.2 | 98.5 | 0.632 | 0.483 | 0.826 | 36. |

Analysis by actual dose normalized to bodyweight:

| | | | | | | | |
|---|---|---|---|---|---|---|---|
| 600 mg GBR830 | AU | 5144 | 7421 | 0.693 | 0.552 | 0.871 | 31. |
| 75 mg GBR830 SC | AU | 4755 | 7421 | 0.641 | 0.507 | 0.81 | 31. |

AUC_{0-∞} = AUC from time 0 to infinity; CI = confidence interval; CV = coefficient of variation; LS means = least square means; SC = subcutaneous. [symbol]
indicates data missing or illegible when filed

Statistical/Analytical Issues

In FIG. 6, geometric mean of PK parameters is presented by treatment/ADA positive and ADA negative subjects separately, rather than by treatment, in order to have better visual clarity in the plots.

Adjustments for Covariates

There were no adjustments for covariates or prognostic factors.

Handling of Dropouts or Missing Data

The serum PK parameters were estimated from the concentration-time profiles for all PKAS subjects. In estimating the PK parameters, BQL values at the beginning of the profile were set to zero. Values that were BQL and occurred after the first quantifiable point were considered missing. Values that were embedded between BQLs, or quantifiable values occurring after 2 or more BQLs, were set to missing at the discretion of the pharmacokineticist. Actual sampling times, rather than scheduled sampling times, were used in all computations involving sampling times. If the actual time or dose time was missing, the scheduled time was substituted in order to calculate the PK parameter.

Subject 9 in the 600 mg GBR830 IV group had an unscheduled PK sample on Day 12, but missed 2 other samples on Day 7 and Day 8. The result for Day 12 was not used in the descriptive statistics of concentrations, but was included in the derivation of PK parameters, using the actual time of the sample.

Subject 26 in the 75 mg GBR830 SC group had serum concentration data available only up to 336 hours post dosing. This subject missed 3 PK samples scheduled for Day 29, Day 43, and Day 71, and the sample for Day 57 was taken but was <LLOQ. Therefore, the PK parameters relying on the terminal elimination phase i.e., CL/F, Vz, AUC_{0-∞}, and VA, were not calculable for this subject.

For Subject 19 in the 600 mg GBR830 IV group and Subject 22 in the 75 mg GBR830 SC group, the PK parameters relying on the terminal elimination were calculated but were not included in the descriptive statistics, as these were considered less reliable based on the R² adjusted values.

Discussion of Pharmacokinetics Results

This study was conducted to investigate absolute bioavailability of GBR830 after single SC administration of GBR830 75 or 600 mg. As reference, single IV administration of 600 mg GBR830 was used.

Absorption Phase:

Following a 1-hour IV infusion of GBR830, Cmax was achieved at early time points (1.00 h to 6.00 h relative to start of infusion) and then declined in a biphasic manner with a distribution phase followed by a relatively longer elimination phase.

Following SC injection, GBR830 was absorbed slowly with gradual increase in serum concentrations and Cmax was reached at around 4 to 5 days (median tmax) (range: 48 h to 192 h). When compared at the same dose level (600 mg), Cmax after SC injection estimated to be approximately 3.2-fold lower than Cmax after IV infusion.

Distribution Phase:

Following IV infusion, GBR830 profiles showed a short distribution phase (about 24 h to 48 h). The volume of distribution (Vss=5.03 L) estimate for GBR830 suggested limited distribution.

Elimination Phase:

The elimination phase after SC injection and IV infusion at 600 mg appeared to be parallel to each other. The geometric mean t1/2 was generally comparable between SC and IV infusion and ranged from 11 days to 15 days. The geometric mean CL after IV infusion was 10.2 mL/h (0.0102 L/h), indicating slow clearance of GBR830.

Absolute Bioavailability:

The Cmax as well as the AUC0-∞, increased in a dose proportional manner from 75 mg to 600 mg GBR830 SC. The absolute bioavailability after SC injection was estimated from both dose corrected AUC0-∞ as well as dose- and body-weight normalized AUC0-∞. The absolute bioavailability estimates were comparable between the two different methods as mentioned above, and also appeared to be comparable between the dose levels. The mean absolute bioavailability (average of both SC dose levels) was 64.7% when corrected only for dose. The geometric mean Vss after IV infusion was around 5.03 L, indicating limited distribution of GBR830.

Between-Subject Variability:

The between-subject variability, as evaluated by geometric CV %, in the Cmax and AUC0-∞ after IV infusion at 600 mg were 17.5% and 16.2%, respectively. After an SC injection of 600 mg GBR830, the between-subject variability in Cmax and AUC0-∞ was 32.3% and 32.4%, respectively. After an SC injection of 75 mg GBR830, the between-subject variability in Cmax and AUC0-∞ was 47.1% and 48.4%, respectively. Overall, the between-subject variability was lower with IV administration compared to the between-subject variability observed after SC administration, as evaluated by geometric CV % (Table 75).

Pharmacokinetics Conclusions

The AUC was lower with SC administration than with IV administration. The absolute bioavailability of GBR830 after a single SC injection of 75 mg GBR830 or 600 mg GBR830 appears to be independent of the doses investigated. The average absolute bioavailability was estimated to be around 65%. GBR830 showed limited distribution (Vss=5.03 L), slow clearance (0.0102 L/h) and a terminal elimination half-life (11 to 15 days). With SC administration, the median tmax ranged between 4 to 5 days with individual subject tmax ranging from 2 to 8 days. Overall, GBR830 PK profile after IV infusion and SC injection was in agreement with the general characteristics of mAbs. When compared at the same dose level, the Cmax after SC injection was approximately 3.2-fold lower than the IV infusion. The Cmax and AUC after SC injection appeared to increase in a dose proportional manner over the range of 75 mg to 600 mg.

Immunogenicity Results

Analysis of Immunogenicity

Blood samples were collected as per the scheduled time points in the protocol. The serum samples were analyzed for ADA formation using a validated ELISA method as previously described, briefely, the antibody GBR 830 (IgG) in calibration standard samples, quality control samples and validation samples was captured by the antigen hOX40-His coated on a 96-well plate. Fc_{γ} fragment specific peroxidase-conjugated Affinipure goat anti-human IgG antibody binds to GBR 830 immobilised on the plate by antigen. Upon addition of tetramethylbenzidine (TMB) substrate a blue colour was developed. Sulphuric acid was added to stop the reaction. The absorbance is measured at 450 nm (Ref. 620 nm).

Serum samples evaluated for ADA formation at any time point were considered ADA positive if the results for both screening and confirmatory assays were positive. Subjects were considered positive for ADA formation if ADA was detected at any time point postdose. The number and percent incidence of ADA (positive and negative) were summarized as described in the SAP.

600 mg GBR830 IV and 600 mg GBR830 SC:

In both the IV and SC 600 mg GBR830 dosing groups, only 1 subject in 600 mg GBR830 SC group tested positive for ADA at predose (Day 1). There was a low incidence of ADA formation in the 600 mg GBR830 IV treatment group, with only 1 out of 10 (10%) subjects tested positive for ADA. In the corresponding GBR830 SC treatment group, there was also only 1 out of 15 (7%) subject tested positive for ADA. In both these subjects, titers ranged between 20 and 40.

75 mg GBR830 SC:

In the 75 mg GBR830 SC treatment group, Subject 26 was positive for ADA at predose (Day 1) with a titer of 10; by Day 15, the titer increased to 40, indicating a treatment-boosted immune response. Hence this subject was considered as ADA positive. The incidence of subjects with a positive ADA result increased during the course of the study, with 10 (67%) subjects overall having positive results (Table 77). In subjects who were positive for ADA, titers ranged between 10 and 640.

TABLE 77
Summary of Immunogenicity (Anti-drug Antibody)
Results (Safety Analysis Set).

| | 600 mg | | | | 600 mg | | | | 75 mg | | | |
|---|---|---|---|---|---|---|---|---|---|---|---|---|
| | GBR830 IV | | | | GBR830 SC | | | | GBR830 SC | | | |
| | (N = 10) | | | | (N = 15) | | | | (N = 15) | | | |
| | Positive | | Negative | | Positive | | Negative | | Positive | | Negative | |
| Visit | n | % | n | % | n | % | n | % | n | % | n | % |
| Day 1 | 0 | 0 | 10 | 100 | 1 | 7 | 14 | 93 | 1 | 7 | 14 | 93 |
| Day 15 | 1 | 10 | 9 | 90 | 0 | 0 | 15 | 100 | 6 | 40 | 9 | 60 |
| Day 29 | 0 | 0 | 10 | 100 | 0 | 0 | 15 | 100 | 3 | 21 | 11 | 79 |
| Day 43 | 0 | 0 | 10 | 100 | 0 | 0 | 15 | 100 | 3 | 21 | 11 | 79 |
| Day 71 | 0 | 0 | 10 | 100 | 1 | 7 | 14 | 93 | 8 | 62 | 5 | 38 |
| Overall^{a} | 1 | 10 | 9 | 90 | 1 | 7 | 14 | 93 | 10 | 67 | 5 | 33 |

^{a}Subjects were counted positive if at any time point postdose they became positive.
% = number of subjects with a positive/negative result as a percentage of number of observations per time point; ADA = anti-drug antibody; IV = intravenous; SC = subcutaneous.
Note:
A sample was considered as positive for ADA if the results for both, screening and confirmatory assays, were positive.

Association Between ADA and PK

The impact of ADA on the PK of GBR830 was evaluated visually by comparing the CL(/F) of ADA-positive subjects to CL(/F) of ADA-negative subjects, stratified by treatment. In addition, dose- and bodyweight-normalized Cmax, AUC0-t and AUC0-∞ were also compared between ADA-positive subjects and ADA-negative subjects, stratified by treatment.

600 mg GBR830 IV and 600 mg GBR830 SC:

Only 1 subject in each treatment arm had detectable anti-GBR830 antibody; hence no meaningful conclusion on the effect of ADA on GBR830 could be derived.

75 mg GBR830 SC:

There were 10 ADA-positive subjects and 5 ADA-negative subjects. There was higherinter-subject variability in the CL/F and other evaluated PK parameters in ADA-positive subjects than in ADA-negative subjects.

As seen in the FIG. 6, ADA-positive subjects did not show a consistent trend of higher clearance compared to the clearance in ADA-negative subjects. There was considerable overlap in the individual subject clearances between the 2 groups. Similarly, ADA-positive subjects did not show a consistent trend of lower serum exposures compared to ADA-negative subjects, with considerable overlap in individual subject serum exposures between the 2 groups. These indicate that there was no major impact of ADA on PK in this study (FIG. 6).

Statistical/Analytical Issues
Subject 26 (75 mg GBR830 SC) had a predose titer of 10 and a postdose titer of 40. This subject was considered ADA-positive. All PK parameters, particularly those depended on the terminal phase could not be derived reliably for this subject
For Subject 22 (75 mg GBR830 SC), who had positive ADA titers on Day 15 through end of study, PK parameters derived based on the terminal phase were less reliable and were excluded from statistical analyses.
Subject 36 (600 mg GBR830 SC) had a predose ADA titer of 20 and the remaining 4 time points postdose tested negative for ADA. This subject was considered ADA-negative due to lack of treatment boosted immune response.

Discussion of Immunogenicity Data

A total of 12 out of 40 subjects were tested positive for ADA in the study. The majority of subjects (10 out of 12) were of 75 mg GBR830 SC (lower dose level); 1 ADA-positive subject was treated with 600 mg GBR830 SC and 1 ADA-positive subject was treated with 600 mg GBR830 IV.

Immunogenicity Conclusions

The incidence of formation of anti-GBR830 antibody was the highest in 75 mg GBR830 SC treatment group (67%), in comparison to that of 600 mg GBR830 IV (10%) and 600 mg GBR830 SC (7%). The presence of ADA may affect the PK of the mAb by increasing the clearance of the mAb and thereby reducing the serum exposures in ADA-positive subjects. The impact of anti-GBR830 antibody on the PK was evaluated visually by plotting the CL(/F), Cmax, AUC0-t and AUC0-∞. The analysis indicated that there was higher variability in the CL/F and other PK parameters in ADA-positive subjects relative to that in ADA-negative subjects. Visual comparison of the PK parameters between ADA-positive and

ADA-negative subjects in 75 mg GBR830 SC treatment group indicated that there is no consistent trend of higher clearance or lower serum exposures in ADA-positive subjects in comparison to that in ADA-negative subjects. This indicates that ADA generation against GBR830 had no major impact on the PK of GBR830 in this study. No AEs were observed related to immunogenicity, indicating no effect of ADA on safety parameters.

Safety Evaluation

Safety evaluations were analyzed using the SAF.

Extent of Exposure

All subjects received study medication; study drug was administered as planned to 39 of the 40 randomized subjects. Drug administration was stopped for 2 intervals (5 second duration and 3 second duration) during infusion for Subject 32 (600 mg GBR830 IV); however, full dose was administered. Infusion was discontinued prematurely for Subject 34 (600 mg GBR830 IV) due to an AE.

Adverse Events

Brief Summary of Adverse Events

All subsequent presentations and discussions of AE data relate to TEAEs, defined as any new AEs or worsening of an existing condition after administration of the study drug up to and including the follow-up visit. Of the 40 subjects randomized to study treatment, 32 (80%) experienced a total of 90 TEAEs (Table 78).
7 of 10 (70%) subjects who received 600 mg GBR830 IV
14 of 15 (93%) subjects who received 600 mg GBR830 SC
11 of 15 (73%) subjects who received 75 mg GBR830 SC No subjects discontinued the study due to AEs.

The most frequently reported TEAEs during the treatment period are presented in Table 79.

TABLE 78
Summary of Treatment-emergent Adverse
Events (Safety Analysis Set).

| | 600 mg | | 600 mg | | 75 mg | | | |
|---|---|---|---|---|---|---|---|---|
| | GBR830 IV | | GBR830 SC | | GBR830 SC | | Total | |
| | (N = 10) | | (N = 15) | | (N = 15) | | (N = 40) | |
| | E | n (%) | E | n (%) | E | n (%) | E | n (%) |
| All TEAEs | 20 | 7 | 41 | 14 (93) | 29 | 11 | 90 | 32 |
| All SAEs | 0 | 0 | 0 | 0 | n/a | 1 (7) | n/a | 1 (3) |
| Withdrawals | 0 | 0 | 0 | 0 | n/a | 2 (13) | n/a | 2 (5) |
| Due to AE | 0 | 0 | 0 | 0 | 0 | 0 | 0 | 0 |
| Not due | 0 | 0 | 0 | 0 | n/a | 2 (13) | n/a | 2 (5) |
| to AE | | | | | | | | |
| Deaths | 0 | 0 | 0 | 0 | 0 | 0 | 0 | 0 |

% = number of subjects (n) as a percentage of number of subjects (N) per treatment; AE = adverse event; E = number of AEs; IV = intravenous; N = number of subjects exposed; n = number of subjects that experienced the AE; n/a = not applicable; SAE = serious adverse event; SC = subcutaneous; TEAE = treatment emergent adverse event.

Out of the total reported 90 TEAEs, 89 TEAEs were reported as mild and only 1 TEAE was reported as moderate (75 mg GBR830 SC). No severe TEAEs were reported. The most commonly reported TEAE was headache (14 TEAEs, 10 (25%) subjects). One related SAE of neuralgic amyotrophy was reported in Subject 22 (75 mg GBR830 SC). No deaths occurred in this study. A total of 12 TEAEs were considered to be related to treatment: 7 TEAEs in the 600 mg GBR830 IV group, 2 TEAEs in the 600 mg GBR830 SC group, and 3 TEAEs in the 75 mg GBR830 SC group.

TABLE 79
Summary of Subjects with the Most Frequently Reported. Treatment-
emergent Adverse Events (Safety Analysis Set, ≥5% Total Subjects).

| | 600 mg | | 600 mg | | 75 mg | | | |
|---|---|---|---|---|---|---|---|---|
| | GBR830 IV | | GBR830 SC | | GBR830 SC | | Total | |
| System Organ Class | (N = 10) | | (N = 15) | | (N = 15) | | (N = 40) | |
| Preferred Term^{a} | E | n (%) | E | n (%) | E | n (%) | E | n (%) |

Nervous system disorders

| | | | | | | | | | | | |
|---|---|---|---|---|---|---|---|---|---|---|---|
| Headache | 7 | 5 (50) | 3 | 3 | (20) | 4 | 2 | (13) | 14 | 10 | (25) |

General disorders and administration site conditions

| Injection Site Haematoma | 0 | 0 | 3 | 3 | (20) | 2 | 2 | (13) | 5 | 5 | (13) |
|---|---|---|---|---|---|---|---|---|---|---|---|
| Injection Site Erythema | 0 | 0 | 1 | 1 | (7) | 1 | 1 | (7) | 2 | 2 | (5) |
| Injection Site Pain | 0 | 0 | 3 | 3 | (20) | 1 | 1 | (7) | 4 | 4 | (10) |
| Vessel Puncture | 1 | 1 (10) | 1 | 1 | (7) | 0 | 0 | | 2 | 2 | (5) |
| Site Haematoma | | | | | | | | | | | |
| Influenza Like Illness | 0 | 0 | 1 | 1 | (7) | 1 | 1 | (7) | 2 | 2 | (5) |

Gastrointestinal disorders

| Abdominal Pain | 0 | 0 | 3 | 3 | (20) | 1 | 1 | (7) | 4 | 4 | (10) |
|---|---|---|---|---|---|---|---|---|---|---|---|
| Diarrhoea | 0 | 0 | 3 | 3 | (20) | 0 | 0 | | 3 | 3 | (8) |
| Aphthous Ulcer | 0 | 0 | 1 | 1 | (7) | 1 | 1 | (7) | 2 | 2 | (5) |

| | | | | | | | | | | |
|---|---|---|---|---|---|---|---|---|---|---|
| Dyspepsia | 0 | 0 | 2 | 2 | (13) | 0 | 0 | 2 | 2 | (5) |

Infection and infestations

| | | | | | | | | | | | |
|---|---|---|---|---|---|---|---|---|---|---|---|
| Viral upper respiratory | 1 | 1 (10) | 2 | 2 | (13) | 3 | 3 | (20) | 6 | 6 | (15) |
| tract infection | | | | | | | | | | | |

Skin and subcutaneous tissue disorders

| | | | | | | | | | | |
|---|---|---|---|---|---|---|---|---|---|---|
| Erythema | 2 | 2 (20) | 1 | 1 | (7) | 0 | 0 | 3 | 3 | (8) |
| Acne | 1 | 1 (10) | 1 | 1 | (7) | 0 | 0 | 2 | 2 | (5) |

Musculoskeletal and connective tissue disorders

| | | | | | | | | | | | |
|---|---|---|---|---|---|---|---|---|---|---|---|
| Myalgia | 0 | 0 | 2 | 2 | (13) | 1 | 1 | (7) | 3 | 3 | (8) |

| | | | | | | | | | | |
|---|---|---|---|---|---|---|---|---|---|---|
| Back pain | 1 | 1 (10) | 1 | 1 | (7) | 0 | 0 | 2 | 2 | (5) |

Respiratory, thoracic and mediastinal disorders

| | | | | | | | | | | | |
|---|---|---|---|---|---|---|---|---|---|---|---|
| Oropharyngeal pain | 0 | 0 | 2 | 2 | (13) | 1 | 1 | (7) | 3 | 3 | (8) |

Renal and urinary disorders

| | | | | | | | | | | | |
|---|---|---|---|---|---|---|---|---|---|---|---|
| Pollakiuria | 0 | 0 | 1 | 1 | (7) | 1 | 1 | (7) | 2 | 2 | (5) |

^{a}Adverse events are coded using MedDRA version 20.0.
% = number of subjects (n) as a percentage of number of subjects (N) per treatment; E = number of AEs; IV = intravenous; MedDRA = medical dictionary for regulatory activities; SC = subcutaneous.

Analysis of Adverse Events

Treatment-Emergent Adverse Events

The incidence of TEAEs was similar across all 3 treatment groups (all GBR830 treatments, 80% of subjects). The incidence of TEAEs was highest in the 600 mg GBR830 SC treatment group (93% of subjects), compared to the 600 mg GBR830 IV treatment group (70% of subjects) and the 75 mg GBR830 SC treatment group (73% of subjects). The most frequently reported TEAE in 25% or greater of all subjects was headache. One (Subject 36; 600 mg GBR830 IV) of these subjects had 3 episodes of headache that were considered related to study drug. This TEAE was reported in all treatment groups, with the greatest number of subjects reporting headache occurring in the 600 mg GBR830 IV group (7 instances of headache in 5 (50%) subjects). The SOC with the most frequently reported TEAEs was general disorders and administration site conditions, with 43% of subjects overall reporting TEAEs in this SOC. Subjects in all treatment groups reported TEAEs in this SOC, with 9 subjects (60%) reporting 12 incidences of TEAEs in the 600 mg GBR830 SC group, 6 subjects (40%) reporting 7 incidences of TEAEs in the 75 mg GBR830 SC group, and 2 subjects (20%) reporting 2 incidences of TEAEs in the 600 mg GBR830 IV group.

Treatment-Related Treatment-Emergent Adverse Events

Treatment-related TEAEs in any treatment group are presented in Table 80. The overall incidence of treatment-related TEAEs was low (10% of all subjects) and similar across all treatment groups. The most frequently reported treatment-related TEAE was aphthous ulcer (5% of subjects). In the 600 mg GBR830 IV treatment group, 1 subject (Subject 34) experienced an infusion-related reaction of generalized urticaria. The TEAE was determined by the Investigator to be related to study drug and mild in severity. The study drug was withdrawn and the AE resolved on the same day.

TABLE 80
Summary of Subjects with Treatment-related, Treatment-
emergent Adverse Events (Safety Analysis Set).

| | 600 mg | | 600 mg | | 75 mg | | | |
|---|---|---|---|---|---|---|---|---|
| | GBR830 IV | | GBR830 SC | | GBR830 SC | | Total | |
| Preferred | (N = 10) | | (N = 15) | | (N = 15) | | (N = 40) | |
| Term ^{a} | E | n (%) | E | n (%) | E | n (%) | E | n (%) |
| Total subjects | 7 | 1 | 2 | 2 | 3 | 1 (7) | 12 | 4 (10) |
| Aphthous Ulcer | 0 | 0 | 1 | 1 (7) | 1 | 1 (7) | 2 | 2 (5) |
| Headache | 3 | 1 | 0 | 0 | 0 | 0 | 3 | 1 (3) |
| Neuralgic | 0 | 0 | 0 | 0 | 1 | 1 (7) | 1 | 1 (3) |
| Amyotrophy | | | | | | | | |
| Acne | 0 | 0 | 1 | 1 (7) | 0 | 0 | 1 | 1 (3) |
| Erythema | 1 | 1 | 0 | 0 | 0 | 0 | 1 | 1 (3) |
| Urticaria | 1 | 1 | 0 | 0 | 0 | 0 | 1 | 1 (3) |
| Vision Blurred | 1 | 1 | 0 | 0 | 0 | 0 | 1 | 1 (3) |
| Injection | 0 | 0 | 0 | 0 | 1 | 1 (7) | 1 | 1 (3) |
| Site Erythema | | | | | | | | |
| Infusion | 1 | 1 (10) | 0 | 0 | 0 | 0 | 1 | 1 (3) |
| Related | | | | | | | | |

^{a} Adverse events are coded using MedDRA version 20.0.
% = number of subjects (n) as a percentage of number of subjects (N) per treatment; E = number of AEs; IV = intravenous; MedDRA = medical dictionary for regulatory activities; SC = subcutaneous.

Deaths, Other Serious Adverse Events, and Other Significant Adverse Events

Deaths

No subjects died during this study.

Other Serious Adverse Events

Subject 22 in the 75 mg GBR830 SC treatment group had an SAE (neuralgic amyotrophy) of moderate intensity. The Investigator assessed that there was a reasonable possibility that the SAE was related to the study drug. The Sponsor could not establish or exclude the possibility of cause and effect relationship between administration of GBR830 and the reported event.

Other Significant Adverse Events

Subject 34 in the 600 mg GBR830 IV group received 242 mL of the 273-mL IV infusion volume. The infusion was discontinued early due to development of mild infusion-related reaction (infusion-related reaction and generalized urticaria) that was considered related to study drug.

Analysis and Discussion of Deaths, Other Serious Adverse Events, and Other Significant Adverse Events

No deaths occurred in the course of the study. One SAE occurred during the study. A 47-year-old white female healthy subject randomized to 75 mg GBR830 SC treatment group was diagnosed with neuralgic amyotrophy, which is a neurologic disorder of unknown etiology. As the start of the event had close temporal relationship with dosing, the Investigator assessed that there was a reasonable possibility that the event was related to study drug. The Sponsor could not establish or exclude the possibility of cause and effect relationship between administration of GBR830 and the reported event.

Clinical Laboratory Evaluation

Listing of Individual Laboratory Measurements by Subject and Each Abnormal Laboratory Value

No positive pregnancy test results were obtained in the study.

Laboratory Values Over Time

In all treatment groups, only small mean changes over time in clinical chemistry, hematology, coagulation, and urinalysis parameters were seen. Although no formal tests of statistical significance were performed, qualitative analysis indicates unremarkable variations in hematology parameters over time, including variations in lymphocyte/leukocyte (%), neutrophil/leukocyte (%), and platelets.

A summary of changes from baseline (Day −1) to Day 71 in a panel of hematology values is presented in Table 81.

TABLE 81
Summary of Mean Changes from Baseline to the Day 71 for Selected Hematology Parameters (Safety Analysis Set)

| | 600 mg GBR830 IV | | | | 600 mg GBR830 SC | | | | 75 mg GBR830 SC | | | |
|---|---|---|---|---|---|---|---|---|---|---|---|---|
| | (N = 10) | | | | (N = 15) | | | | (N = 15)^{a} | | | |
| | Day −1 | | Day 71 | | Day −1 | | Day 71 | | Day −1 | | Day 71 | |
| | (BL)^{b} | | (change | | (BL)^{b} | | (change | | (BL)^{b} | | (change | |
| | mean | | from BL) | | mean | | from BL) | | mean | | from BL) | |
| Parameter | (SD) | | mean | | (SD) | | mean | | (SD) | | mean | |
| Basophils/Leukocytes (%) | 0.62 | | −0.03 | | 0.65 | | −0.06 | | 0.43 | | 0.05 | |
| Eosinophils/Leukocytes (%) | 2.92 | | 0.03 | | 2.21 | | 0.40 | | 1.96 | | −0.25 | |
| Erythrocyte Mean | 21.0 | (0.6) | 0.4 | (0.4) | 20.8 | (0.7) | 0.3 | (0.4) | 20.6 | (0.5) | 0.6 | (0.4) |
| Corpuscular Hb | | | | | | | | | | | | |
| Erythrocyte | 8 | (8) | −4 | (5) | 8 | (5) | 0 | (4) | 10 | (7) | −5 | (7) |
| Sedimentation Rate | | | | | | | | | | | | |
| Erythrocytes (10¹²/L) | 5.08 | | −0.19 | | 4.81 | | −0.10 | | 4.80 | | −0.10 | |
| Hematocrit (L/L) | 0.44 | | −0.02 | | 0.42 | | −0.01 | | 0.43 | | −0.01 | |
| Hemoglobin (mmol/L) | 9.3 | (0.6) | −0.1 | (0.3) | 8.8 | (0.6) | −0.1 | (0.4) | 8.9 | (0.9) | 0.0 | (0.4) |
| Lymphocytes/Leukocytes | 36.63 | (11.30) | −1.06 | (10.31) | 36.21 | (6.95) | −4.59 | (6.87) | 30.26 | (9.43) | −0.75 | (9.05) |
| (%) | | | | | | | | | | | | |
| Monocytes/Leukocytes (%) | 6.23 | | −0.63 | | 6.19 | | −0.36 | | 5.81 | | −0.46 | |
| Neutrophils/Leukocytes (%) | 53.62 | (12.49) | 1.69 | (11.75) | 54.75 | (7.49) | 4.58 | (7.70) | 61.55 | (11.20) | 1.42 | (10.58) |
| Platelets (10⁹/L) | 253 | (55) | 21 | (26) | 239 | (43) | 16 | (30) | 229 | (54) | 0 | (40) |

BL = baseline; Hb = hemoglobin; IV = intravenous; SC = subcutaneous; SD = standard deviation.
^{a}Hematology parameters were collected for 13 subjects at both Day −1 and Day 71.
^{b}Baseline is defined as the last observation recorded before the first study drug administration

Individual Subject Changes

For all clinical chemistry, hematology, and urinalysis parameters, the majority of subjects across all treatment groups had values that were normal at both screening and end of treatment. A few shifts were observed to abnormal but were not clinically significant, with similar frequencies at all scheduled laboratory evaluations.

Overall, the most pronounced shifts were in the lipid profile parameters, which were expected to occur in this study, as Visits 3 through Visit 8 were outpatient and the subjects' diet was not uniform. All subjects were requested to fast for at least 4 hours prior to collection of clinical laboratory safety samples. No treatment group had a shift of more than 4 subjects from normal-to-high or normal-to-low in any individual parameter at any single time point; in the 600 mg GBR830 SC group, 4 subjects shifted from normal-to-high for very low density lipoprotein (VLDL) cholesterol. Qualitative analysis indicated shifts from high-to-normal in ESR in all treatment groups from Day −1 to Day 15, Day −1 to Day 43, and Day −1 to Day 71. Although no formal statistical analysis was conducted on these shifts, it should be noted that ESR may be used as a laboratory marker of inflammation. All treatment groups had at least 1 shift from high-to-normal in ESR, with a change from high-to-normal observed in the 75 mg GBR830 SC group, for 4 subjects at Day 43 and for 3 subjects at Day 71.

Individual Clinically Significant Abnormalities

No subjects were reported as having any laboratory parameters (chemistry, hematology, coagulation, and urinalysis) that were clinically significantly abnormal at screening or at any scheduled or unscheduled time points, including the final visit.

Vital Signs, Physical Findings, and Other Observations Related to Safety

Vital Signs

In all treatment groups, only small similar mean changes in vital signs were observed between baseline and the Day 71. A summary of mean changes from screening to Day 71 is provided in Table 82. No subjects had vital signs values considered to be clinically significantly abnormal and no TEAEs related to abnormal vital signs were reported.

TABLE 82
Summary of Mean Changes from Baseline to the
Day 71 for Vital Signs (Safety Analysis Set)

| | 600 mg GBR830 | | 600 mg GBR830 | | 75 mg GBR830 |
|---|---|---|---|---|---|
| | IV (N = 10) | | SC (N = 15) | | SC (N = 15)^{a} |
| Vital Sign | mean (SD) | | mean (SD) | | mean (SD) |
| Diastolic Blood | 3 | (8) | 4 | (5) | 3 (8) |
| Pressure (mmHg) | | | | | |
| Systolic Blood | 3 | (10) | 5 | (11) | 8 (9) |
| Pressure (mmHg) | | | | | |
| Pulse Rate (bpm) | −2 | (11) | 1 | (8) | 5 (6) |
| Temperature | −0.1 | (0.6) | 0.1 | (0.4) | −0.1 (0.5) |
| (° C.) | | | | | |
| Respiratory | 0 | (2) | 3 | (4) | 1 (3) |
| Rate | | | | | |

bpm = beats per minute; C. = Celsius; IV = intravenous; mmHg = millimeters of mercury; min = minute; SC = subcutaneous; SD = standard deviation.
^{a}Vital signs were collected for 13 subjects at both Day −1 and Day 71.
Note:
Baseline is defined as the last observation recorded before the first study drug administration

Electrocardiograms

In all treatment groups, similar mean changes in ECG parameters were observed from Baseline to Day 3 and also from Baseline to Day 71. A summary of mean changes from Baseline to the final visit (Day 71) is provided in Table 83. The proportion of subjects with ECG findings considered to be normal or abnormal but not clinically significant at screening was similar across the treatment groups. Few shifts were observed to abnormal but not clinically significant, in an equal proportion across treatment groups. No TEAEs related to abnormal ECGs were reported.

TABLE 83
Summary of Mean Changes from Baseline to the Day 71 for
Electrocardiogram Parameters (Safety Analysis Set).

| | 600 mg GBR830 | | 600 mg GBR830 | | 75 mg GBR830 | |
|---|---|---|---|---|---|---|
| | IV (N = 10) | | SC (N = 15) | | SC (N = 15)^{a} | |
| ECG Parameter | mean (SD) | | mean (SD) | | mean (SD) | |
| Heart rate (bpm) | 2 | (11) | 1 | (10) | 4 | (7) |
| PR Interval (msec) | −7 | (10) | −9 | (13) | −8 | (9) |
| QRS-Duration (msec) | 2 | (9) | 0 | (5) | −2 | (7) |
| QT Interval (msec) | 8 | (27) | −3 | (24) | −15 | (11) |
| QTc (Fridericia) | 10 | (12) | 0 | (10) | −7 | (11) |
| Interval (msec) | | | | | | |

bpm = beats per minute; ECG = electrocardiogram; msec = millisecond; IV = intravenous; SC = subcutaneous; SD = standard deviation.
^{a}ECG parameters were collected for 13 subjects at both Day −1 and Day 71.
Note:
Baseline is defined as the last observation recorded before the first study drug administration.

Physical Examinations

For all body systems, the majority of subjects had normal or abnormal not clinically significant changes findings upon physical examination. One clinically significant abnormal finding was reported for 1 subject, Subject 22, who was treated with 75 mg GBR830 SC and had an abnormal finding for the lungs at Day 71. The finding was noted as shallow breathing and dyspnea when lying flat. A lack of abdominal distension was noted during inspiration. This subject experienced an SAE of neuralgic amyotrophy related to abnormal physical findings of the lungs (bilateral paralysis of the diaphragm).

Local Tolerability at the Injection Site

For the GBR830 SC groups, local tolerability at the injection site was evaluated by assessing injection site reactions including pain, swelling and redness. Treatment with 600 mg GBR830 SC or 75 mg GBR830 SC, respectively, was administered in the fatty layer of the lower right or left quadrant of the abdomen. The majority of injection site assessments were mild and a few were moderate. No severe injection site assessments were reported. Swelling was not reported by any subjects in either treatment group at any time point for either the right or left abdomen. No clinically significant differences in local tolerability at the injection site were indicated between the 600 mg GBR830 SC or 75 mg GBR830 SC treatment groups.

Safety Conclusions

The safety conclusions of this study are as follows: GBR830 was generally safe and well-tolerated.
Of the 40 subjects randomized to study treatment, 32 (80%) experienced a total of 90 TEAEs: 7 of 10 (70%) subjects who received 600 mg GBR830 IV, 14 of 15 (93%) subjects who received 600 mg GBR830 SC, and 11 of 15 (73%) subjects who received 75 mg GBR830 SC.
There were no appreciable differences in intensity of these events among treatment groups.
TEAEs were generally of mild intensity, with only 1 TEAE reported as being of moderate intensity.
The most frequently reported TEAE in 25% or greater of all subjects was headache. One of these subjects had 3 episodes of headache that were considered related to study drug. This TEAE was reported in all treatment groups, with the greatest number of subjects reporting headache occurring in the 600 mg GBR830 IV group (7 instances of headache, occurring in 5 (50%) subjects).
The SOC with the most frequently reported TEAEs was general disorders and administration site conditions, with 43% of subjects reporting TEAEs in this SOC. Subjects in all treatment groups reported TEAEs in this SOC, with 9 subjects (60%) reporting 12 incidences of TEAEs in the 600 mg GBR830 SC group, 6 subjects (40%) reporting 7 incidences of TEAEs in the 75 mg GBR830 SC group, and 2 subjects (20%) reporting 2 incidences of TEAEs in the 600 mg GBR830 IV group.
The overall incidence of treatment-related TEAEs was low (10% of all subjects) and similar across all treatment groups. The most frequently reported treatment-related TEAE was aphthous ulcer (5% of subjects).
One treatment-emergent SAE (neuralgic amyotrophy) was reported in a subject treated with 75 mg GBR830 SC. The event was considered by the Investigator to be related to study treatment but not related to study procedure.
Study drug (600 mg GBR830 IV) was withdrawn for 1 subject due to infusion-related reaction of mild and related generalized urticaria. The AE resolved on the same day, and the subject went on to complete the study.
No subjects died during the study, and no subjects were withdrawn during the study due to a TEAE.
No clinically relevant treatment-related findings were observed for laboratory evaluations, vital signs, ECGs, physical examinations, or local tolerability findings at injection site.

Discussion and Overall Conclusions

Discussion

GBR830 is a humanized, IgG1 antibody specific for OX40 (CD134), a T cell co-stimulatory molecule responsible for the expansion and maintenance of effector and memory immune responses.

Blocking the binding of OX40 to its ligand OX40L (CD252) consequently reduces the longevity and efficacy of these immune responses and thus gives GBR830 the potential to treat T cell pathology related autoimmune diseases. The present study was designed to evaluate the absolute bioavailability of GBR830 following SC administration in healthy adult subjects as a primary objective. Secondary objectives included the evaluation of PK, immunogenicity, safety, and tolerability after single doses of GBR830 in healthy adult subjects following SC and IV administration.

The absolute bioavailability of GBR830 after the SC injection appeared to be dose independent and the average absolute bioavailability is estimated to be around 65%. The GBR830 serum concentration profile after IV infusion and SC injection was in general agreement with what is anticipated for a mAb. When compared at the same dose level, the Cmax after SC injection was approximately 3.2-fold lower than the IV infusion. The t½ after IV infusion and SC injection were generally similar, around 11 days at 75 mg GBR830 SC and around 15 days at 600 mg (SC and IV). The limited distribution and slow clearance estimated for GBR830 is in agreement with the general characteristics of mAbs.

Anti-drug antibody against GBR830 was detected in all treatment groups. The majority of the subjects tested positive for ADA in the lower dose level (75 mg GBR830 SC). The visual comparison of the PK parameters at 75 mg GBR830 SC between ADA-positive and

ADA-negative subjects indicated that there is no obvious trend of higher clearance or lower serum exposures in ADA-positive subjects in comparison to that in ADA-negative subjects. This indicates that ADA generation against GBR830 had no major impact on the PK of GBR830 in this study. No AEs were observed related to immunogenicity, indicating no effect of ADA on safety parameters.

Safety and tolerability were comparable across all treatment groups, with a similar incidence of TEAEs across treatment groups. Of the 40 subjects randomized to study treatment, 32 (80%) experienced a total of 90 TEAEs: 7 of 10 (70%) subjects who received 600 mg GBR830 IV, 14 of 15 (93%) subjects who received 600 mg GBR830 SC, and 11 of 15 (73%) subjects who received 75 mg GBR830 SC. There were no appreciable differences in intensity of TEAEs among treatment groups; TEAEs were generally of mild intensity, with only 1 TEAE reported as moderate intensity. The overall incidence of treatment-related TEAEs was low (10% of all subjects) and similar across all treatment groups. The number of subjects with local tolerability findings at injection site was low and similar across both SC treatment groups, with no severe tolerability assessments reported at injection site. No clinically relevant treatment-related findings were observed for laboratory evaluations, vital signs, ECGs, and physical examinations.

No deaths occurred during the study. One subject treated with 600 mg GBR830 IV was permanently discontinued from the study drug due to a mild nonserious TEAE (infusion-related reaction [infusion-related reaction and generalized urticaria]). One SAE (neuralgic amyotrophy) of moderate intensity was reported in a subject treated with 75 mg GBR830 SC.

Overall, GBR830 was safe and well-tolerated when administered as IV or SC at single doses in healthy adult subjects.

Overall Conclusions
The absolute bioavailability of GBR830 after a single SC injection of 75 mg GBR830 or 600 mg GBR830 appears to be independent of the doses investigated. The average absolute bioavailability was estimated to be around 65%.
The PK profile of GBR830 after IV infusion and SC injection was in agreement with the general characteristics of mAbs. The Cmax and AUC after SC injection showed a dose proportional increase over the range of 75 mg to 600 mg.
The incidence of anti-GBR830 antibody was the highest in 75 mg GBR830 SC treatment group (67%), in comparison to that of 600 mg GBR830 IV (10%) and 600 mg GBR830 SC (7%). Visual comparison of the PK parameters between ADA-positive and ADA-negative subjects in 75 mg GBR830 SC treatment group indicated that there is no consistent trend of higher clearance or lower serum exposures in ADA-positive subjects in comparison to that in ADA-negative subjects. This indicates that ADA generation against GBR830 had no major impact on the PK of GBR830 in this study.
GBR830 was safe and well-tolerated when administered as IV or SC at single doses in healthy adult subjects.

### Example 3. A Randomized, Double-Blind, Placebo-Controlled, Parallel-Group Study of GBR830 in Adult Subjects with Moderate to Severe Atopic Dermatitis

Atopic dermatitis is considered a polar Th2 disease. Chronic AD lesions have been shown to have a marked increase in Th2 T cells and related cytokines. OX40 mediates signaling by thymic stromal lymphopoietin (TSLP)-activated dendritic cells (DCs) and is highly upregulated in atopic skin. TSLP-activated DCs have been shown to preferentially activate Th2 T-cell responses in autologous and allogeneic cultures in an OX40-dependent manner. Therefore, GBR 830 may hold the promise for a more targeted, effective and less toxic approach to systemic therapy in AD. Preclinical pharmacology studies demonstrated that GBR 830 is able to block the interaction between OX40 and OX40L and suppress T cell proliferation and allogeneic reactions, such as mixed lymphocyte reactions, with 50% effective concentrations ranging from 0.1 to 3 μg/mL. These studies also demonstrated that GBR 830 has antibody-dependent cellular cytotoxicity and complement-dependent cytotoxicity potential.

In summary, AD is a chronic inflammatory disease that is one of the most prevalent disorders worldwide, most traditional therapies are intended for clinical improvement and symptomatic relief without targeting the specific pathways that initiate and promote AD. GBR 830 selectively inhibits OX40 reducing the longevity and efficacy of the related memory T-cell response. This mode of action gives GBR 830 the potential to treat memory T cell pathology related autoimmune diseases including AD. Therefore GBR 830 may hold the promise for a more targeted, effective and better tolerated approach to systemic therapy in AD. In this regard a phase 2a proof-of-concept study in 62 moderate-to-severe AD subjects showed that two doses of GBR 830, 4 weeks apart, were safe, well-tolerated and induced significant, progressive, long-lasting tissue changes including reduced mRNA expression of Th1, Th2 and Th17/22 inflammatory cytokines.

To further assess and increase GBR830 efficacy in adults with moderate-to-severe atopic dermatitis, in the present invention the dosage of GBR830 is optimized. In particular, the present invention includes, a multicenter, double-blind, placebo-controlled study conducted in 3 phases: a screening phase, a treatment phase, and a follow-up phase. Subjects participate in a 16-week treatment period (including 14 weeks of study drug administration) with 12 weeks follow-up from Week 16 to Week 28, if not participating in the open-label extension study.

Study Objectives and Purpose

Primary Objective

To characterize the efficacy of GBR830 monotherapy in adults with moderate-to-severe atopic dermatitis (AD) compared to placebo as measured by Investigator's Global Assessment (IGA).

Secondary Objectives

The key secondary objective is to evaluate the proportion of subjects with EASI 75 (≥75% improvement from baseline) at Week 16. Other secondary objectives:
To evaluate safety, tolerability, PK and immunogenicity of GBR830 monotherapy in adults with moderate-to-severe AD.
To measure the effect of GBR830 on disease activity in adult subjects with moderate-to-severe

AD, as measured by validated tools (EASI response, SCORAD).

Exploratory Objective

To evaluate pharmacodynamics (PD) of GBR830 in adults with moderate-to-severe AD.

Overall Study Design

This is a multicenter, double-blind, placebo-controlled study. The study will be conducted in 3 phases: a screening phase, a treatment phase, and a follow-up phase. Subjects will participate in a 16-week treatment period (including 14 weeks of study drug administration) with 12 weeks follow-up from Week 16 to Week 28.

Subject eligibility will be assessed during screening, which will occur within 28 days prior to randomization. During the screening period, treatments for AD will be withdrawn or modified for the subject. Subjects may be re-screened once (within or outside of the screening period) if they fail the screening evaluation. All screening procedures will be repeated during rescreening. Subjects who continue to meet eligibility criteria will undergo Day 1/baseline (predose) assessments and will be randomized to equal groups of approximately 98 subjects each. Subjects will receive SC injections of GBR830, or corresponding placebo. A total of 8 doses (GBR830 or placebo) will be administered, including a loading dose on Day 1, followed by maintenance dosing from Week 2 until the last dose at Week 14. Each subject will receive a dose on Day 1 (2 injections) and every 2 weeks (q2w) (1 injection per occasion) starting from Day 15 through Week 14, according to the following treatment assignment below:
Group 1: Dose of 600 mg GBR830 (2 SC injections each containing 300 mg in a 2 mL volume) on Day 1, followed by q2w dosing of 300 mg GBR830 (1 SC injection containing 300 mg in a 2 mL volume), starting at Day 15 (Week 2).
Group 2: Dose of 600 mg GBR830 (2 SC injections each containing 300 mg in a 2 mL volume) on Day 1, followed by dosing every 4 weeks (q4w) of 300 mg GBR830 (1 SC injection containing 300 mg in a 2 mL volume) starting at Day 29. In order to maintain blinding, placebo (1 SC injection of 2 mL) will be administered q4w starting at Day 15 (Week 2).
Group 3: Dose of 150 mg GBR830 (2 SC injections each containing 75 mg in a 2 mL volume) on Day 1, followed by q4w dosing of 75 mg GBR830 (1 SC injection containing 75 mg in a 2 mL volume) starting at Day 29. In order to maintain blinding, placebo (1 SC injection of 2 mL) will be administered q4w starting at Day 15 (Week 2).
Group 4: Dose of placebo (2 SC injections of 2 mL volume) on Day 1, followed by q2w dosing with placebo (1 SC injection of 2 mL) starting at Day 15 (Week 2).

All subjects will receive a loading dose consisting of 2 SC injections, and each of the 7 maintenance doses will consist of 1 SC injection per dose, to maintain the blind, as described above and in Table 85. Study assessments will be performed at baseline (Day 1) and every week s until Week 16. An experimental population PK design will be used for the PK blood sampling. The subjects in the rich PK group and will have additional blood sampling between Days 1 to 8 (Week 1), and Days 85 to 92 (Week 12). The subjects in the sparse PK group will have widespread sampling with fewer time points for each subject. Approximately 80 rich PK subjects will be randomized (in a 1:1:1:1 ratio) to the treatment arms. Follow-up visits will be conducted for all subjects every 4 weeks, from Weeks 16 to 28 (at Days 113, 141, 169 and 197). Subjects will be clinically monitored for safety throughout the study, including anaphylactic reactions and/or injection site reactions (ISRs), with monitoring at the study site for 2 hours after the first dose on Day 1 and for 1 hour after dosing on subsequent visits. See FIG. 7 for a schematic diagram of the study design.

Study Rationale

The objective of this study is to investigate the efficacy, safety, PK, and PD of different subcutaneous (SC) dosing regimens of GBR830 in subjects with AD. In this regard a phase 2a proof-of-concept study in 62 moderate-to-severe AD subjects has been conducted to assess safety of subjects as well as biological responses in biopsies of involved skin after receiving 2 doses of 10 mg/kg IV 4 weeks apart (primary endpoints). As secondary endpoints, multiple clinical endpoints were assessed. Two doses of GBR830, 4 weeks apart, were safe, well-tolerated and induced significant, progressive, long-lasting tissue changes including reduced mRNA expression of Th1, Th2 and Th17/22 inflammatory cytokines. Moreover, improved clinical responses in SCORAD, EASI and Investigator's Global Assessment (IGA) were associated with progressive reductions of some key biomarkers. Hyperplasia measures (epidermal thickness, K16 expression and Ki67) showed higher reductions in the GBR830 group (P<0.001). In summary, these data highlighted the potential of GBR830 in AD as suggested by the mode of action.

Dosing Rationale

Pharmacokinetic and efficacy data in subjects with moderate-to-severe AD, PK and safety data after SC injection and IV infusion to healthy subjects (Study GBR830-102), and PK and safety data after IV infusion up to 10 mg/kg (Study GBR830-101), available safety data from the ongoing phase 1 study (Study GBR830-103), and the receptor occupancy data of GBR830 in activated human whole blood were considered in determination of the dosage regimen for the current GBR830-204 study.

Based on the available results, GBR830 is safe and well tolerated up to 40 mg/kg dose level and showed dose proportional PK across the evaluated dose range (0.3 mg/kg to 40 mg/kg). The absolute bioavailability of GBR830 after SC injection is approximately 65%. The average t_{1/2} of GBR830 ranged from 10 to 15 days, and appeared to be independent of dose level or route of administration. Receptor occupancy experiment with GBR830 in activated human whole blood indicated that maximum receptor occupancy (ROₘₐₓ) was achieved at a concentration of approximately 25 μg/mL of GBR830 and a 50% receptor occupancy (R050) was achieved at a concentration of around 3 μg/mL of GBR830. In study GBR830-201, the average C_{trough} was maintained at around 30 μg/mL over the entire dosing interval, similar to the concentration required for ROₘₐₓ.

The dosing schedule for the current study includes a loading dose followed by maintenance dosing for the GBR830 treatment arms (Groups 1, 2 and 3). The loading dose for each group is selected based on the corresponding maintenance dose and the dosing frequency in order to achieve steady state levels faster. The same regimen will be followed for the placebo arm (Group 4) to maintain the blind.
The highest GBR830 dosage regimen (Group 1) includes a 600 mg GBR830 SC loading dose followed by 300 mg GBR830 SC q2w maintenance dosing. At this dose level, the average steady state C_{trough} is anticipated to be approximately 46 μg/mL, which is slightly higher than what was achieved in the GBR830-201 study. With this dosage regimen, the average steady state C_{trough} values will be maintained above the concentration that elicited ROₘₐₓ in the in vitro experiments and therefore has been selected as the highest dose for the current study.
The middle GBR830 dosage regimen (Group 2) includes a 600 mg GBR830 SC loading dose followed by 300 mg GBR830 SC q4w maintenance dosing. At this dose level, the average steady state C_{trough} is anticipated to be approximately 16 μg/mL, which is less than what is required for ROₘₐₓ, yet greater than the level that elicited RO50 in the in vitro experiments and therefore has been selected as the middle dose.
The lowest GBR830 dosage regimen (Group 3) includes a 150 mg GBR830 SC loading dose followed by 75 mg GBR830 SC q4w maintenance dosing. The average C_{trough} anticipated with this regimen is approximately 4.0 μg/mL, similar to the concentration required to elicit R050, in in vitro experiments.

With the above GBR830 dosage regimens, an approximate 10-fold spread in the average steady state C_{trough}, and approximate 8-fold spread in average steady state AUC_{4week} are expected and are considered adequate to meet the objectives of the study.

Estimated Duration of Subject Participation

The anticipated maximum total study duration for each subject is approximately 32 weeks. This duration will consist of the screening period of up to 4 weeks, the treatment period of up to 16 weeks (14 weeks of treatment, with a loading dose on Day 1 followed by q2w maintenance dosing from Day 15 [Week 2] until the last dose at Week 14), and the follow-up period of at least 12 weeks starting at the end of the treatment period (i.e., Week 16 to Week 28).

Number of Subjects

Approximately 563 subjects will be screened to randomize 392 eligible subjects in a 1:1:1:1 ratio. The randomization will be stratified by severity (moderate or severe, as assessed by IGA), geographic region (North America vs European Union), and subjects consenting to rich PK sampling (Yes/No).

Subject Inclusion and Exclusion Criteria

Subject inclusion criteria include:
1. Provide written informed consent and any locally required authorization prior to any protocol-related procedures, including screening evaluations.
2. Willing and able to comply with all aspects of the protocol.
3. Male or female ≥18 years at the time of screening.
4. Physician diagnosis of AD for >1 year; diagnosis of AD as defined by the American Academy of Dermatology Consensus Criteria (Eichenfeld et al, 2014).
5. AD involvement of ≥10% body surface area (BSA) at screening and baseline.
6. EASI score of ≥12 at screening or ≥16 at baseline.
7. IGA score of at screening and baseline (on the 0 to 4 IGA scale, in which 3 is moderate and 4 is severe).
8. Baseline Pruritus Numerical Rating Scale (NRS) score for maximum itch intensity over the previous 24 hours.
9. Documented/reported recent history (within 6 months before the screening visit) of inadequate response to treatment with topical medications (topical corticosteroids or crisaborole or topical calcineurin inhibitors) or subjects for whom topical treatments are otherwise medically inadvisable (eg, because of important side effects or safety risks as defined in the protocol). Documents may include medical records, physician to healthcare provider communication (with date and time of communication and physician signature) or pharmacy records (with clearly listed dates of dispensation). A course of marketed systemic immunosuppressants (eg, prednisone, cyclosporine, methrotrexate) for AD in the 6 months prior to screening assumes failure of topicals and is acceptable in place of topical failure.
10. Have applied a stable dose of topical emollient (moisturizer) twice daily for at least the 7 consecutive days immediately before the baseline visit (with the exception of prohibited moisturizers containing additives listed in Exclusion Criteria #5).
11. Must agree to the following requirements during the study:
   a. If female and of childbearing potential, she must have a negative serum pregnancy test result within 7 days prior to first dosing and a negative urine pregnancy test predose on Day 1. She must be willing to use a highly effective form of contraception ((ICH E8 Guideline, 1997; ICH M3[R2] Guidance, 2009; FDA M3[R2] Guidance, 2010) for the duration of the study and for at least 3 months after the last dose of study medication. Methods like periodic abstinence, post ovulation procedures and withdrawal are not considered adequate. Each woman is considered to have childbearing potential unless she has been surgically sterilized by hysterectomy or bilateral tubal ligation/salpingectomy or has been post-menopausal for at least 2 years. For postmenopausal women only, follicle stimulating hormone (FSH) testing is performed at screening to confirm non-childbearing potential (FSH 40 IU/L).
   b. If male with a partner of childbearing potential, he must be willing to use condoms in combination with a second effective method of contraception during the study. Each man is considered as potent unless surgically sterilized (with appropriate post vasectomy documentation of the absence of sperm in the ejaculate). Male subjects must continue to use contraception for 180 days following administration of the study drug.
   c. Male subjects should agree not to donate sperm during the study and for 180 days following administration of the study drug.

Subject Exclusion Criteria Include:
1. Prior treatment with GBR830.
2. Employee of the clinical study site or any other individuals involved with the conduct of the study, or immediate family members of such individuals.
3. Concurrent enrollment in another investigational clinical study.
4. Treatment with any of the following before baseline:
   a. Investigational biological agent within 8 weeks of baseline or 5 half-lives, whichever is longer.
   b. Investigational drugs, eg, phosphodiesterase type 4 (PDE4) inhibitors, Janus kinase (JAK) inhibitors, within 4 weeks of baseline.
   c. Phototherapy for AD within 4 weeks of baseline.
   d. Marketed drugs, including systemic corticosteroids, immunosuppressive/immunomodulatory drugs including but not limited to cyclosporine, mycophenolate-mofetil, interferon-gamma (IFN-γ), PDE4 inhibitors, JAK inhibitors, azathioprine or methotrexate, within 4 weeks of baseline.
   e. Topical medications, including corticosteroids, tacrolimus, and/or pimecrolimus and crisaborole within 1 week of baseline.
   f. Regular use (>2 visits/week) of a tanning booth/parlor within 4 weeks of baseline.
   g. Biologics, depending on the type of biologic such as cell-depleting agents including but not limited to rituximab: within 6 months of baseline, or until lymphocyte and CD19+ lymphocyte count returns to normal, whichever is longer.
   h. Biologics including infliximab, adalimumab, golimumab, certolizumab pegol, abatacept, etanercept, anakinra, dupilumab: within 12 weeks of baseline, or 5 half-lives, whichever is longer.
   i. Other biologics: within 5 half-lives (if known).
5. Initiation of treatment of AD with prescription moisturizers or moisturizers containing additives such as ceramide, hyaluronic acid, urea, or filaggrin degradation products during the screening period (subjects may continue using stable doses of such moisturizers if initiated before the screening visit).
6. Planned or anticipated use of any prohibited medications and procedures during study treatment as defined in the study protocol.
7. Subjects who are immunocompromised (congenital or acquired), or who have had a recent (within 3 months prior to baseline) or current serious systemic infection (including infectious mononucleosis-like illness or herpes zoster).
8. Treatment with a live (attenuated) vaccine within 12 weeks before the baseline visit.
9. Subjects who have evidence of active or latent tuberculosis (TB) as documented in their medical history, or test positive at screening. For indeterminate cases an informed decision will be made between Principal Investigator and Medical Monitor.
10. Active chronic or acute infection requiring treatment with systemic antibiotics, antivirals, antiparasitics, antiprotozoals, or antifungals within 2 weeks before the baseline visit, or superficial skin infections within 1 week before the baseline visit. NOTE: subjects may be rescreened after infection resolves.
11. Presence of skin comorbidities that may interfere with study assessments, in the opinion of the Investigator, including subjects with psoriasis.
12. Poorly controlled asthma as assessed by the Asthma Control Questionnaire [ACQ-5] based on International ERS/ATS guidelines.
13. Any condition at baseline which is part of the criteria for discontinuation of study drug as defined in the study protocol.
14. Subjects who are known to be seropositive for human immunodeficiency virus (HIV) or who test positive at screening.
15. History of a positive result for Hepatitis B surface antigen (HBsAg), antibody to Hepatitis B core antigen (anti-HBcAg), or antibody to Hepatitis C virus (anti-HCV) or presence of and of these findings at screening.
16. History of alcohol or drug abuse within 2 years of the screening visit.
17. Subjects with a history of non-malignant lymphoproliferative disorders, or a history of malignancy within 5 years before baseline (except completely treated in situ cervical carcinoma or non-metastatic squamous or basal cell carcinoma of the skin).
18. In the opinion of the Investigator, subjects with any other medical or psychological condition as well as laboratory values, which are significantly different from normal reference ranges and/or judged to be clinically significant; and/or any condition that would interfere with evaluation of the study drug or interpretation of subject safety or study results, including conditions that are inadequately understood at the time of screening.
19. Subjects with a history of substance abuse or dependence that in the opinion of the Investigator, is considered to interfere with the subject's participation in the study.
20. Planned or anticipated major surgical procedure during the subject's participation in this study.
21. Women who are pregnant or breast feeding.

Subject Withdrawal Criteria

A subject may voluntarily discontinue study participation at any time after giving informed consent and before the completion of the last visit of the study. Subjects may also be withdrawn from study drug treatment at the discretion of the Investigator or Sponsor for safety, noncompliance, or administrative reasons.

The reasons for subject withdrawal will be recorded and may include, but are not limited to:
1. Withdrawal of consent by the subject to continue in the study. If consent is withdrawn, the subject will not receive any further investigational product (IP) or further study observation. Note that the subject may need to undergo additional tests or tapering of treatment to withdraw safely.
2. Development of a serious or intolerable adverse event (AE) that necessitates discontinuation at the discretion of the Investigator including but not limited to:
   Anaphylactic reaction or other severe systemic reaction to study drug injection
   Diagnosis of a malignancy (new or relapse) during study, excluding carcinoma in situ of the cervix, or squamous or basal cell carcinoma of the skin.
   Any infection that is opportunistic, such as active TB and other infections whose nature or course may suggest an immuno-compromised status.
   An infection that requires parenteral treatment with antibiotic, antifungal, antiviral, anti-parasitic, or anti-protozoal agents or requires oral treatment with such agents for longer than 2 weeks.
3. Severe laboratory abnormalities:
   Serum creatinine >1.5 mg/dL (equivalent to Systeme International (SI) unit of >114.39 μmol/L).
   Alanine aminotransferase (ALT) and/or aspartate aminotransferase (AST) values >3× upper limit of normal (ULN) with total bilirubin >2×ULN (unless elevated bilirubin is related to confirmed Gilbert's Syndrome).
   Absolute neutrophil count ≤500/μL (equivalent to SI unit of ≤0.5×109/L) or absolute lymphocyte count ≤600/μL (equivalent to SI unit of ≤0.6×109/L) or platelet count ≤50,000/μL (equivalent to SI unit of 50×109/L) or any abnormal evaluations which in the opinion of the Investigator is clinically significant.
   Confirmed AST and/or ALT >5×ULN (for more than 2 weeks).
4. At the discretion of the Investigator, when he/she believes continued participation is not in the best interest of the subject.
5. At the discretion of the Investigator, when the subject does not adhere to the study procedures.
6. A positive pregnancy test.
7. A female partner of a male study subject becomes pregnant.
8. A protocol deviation that, in the opinion of the Sponsor and Investigator, warrants discontinuation from the study.
9. Rescue treatment for AD (drug or phototherapy or both) given based on the opinion of the Investigator; subjects who receive rescue treatment will be discontinued from study drug, but asked to remain in the study and complete all visits and assessments through the End of Study visit (Day 197).

A subject who is withdrawn from study drug treatment will be asked to complete all procedures scheduled for the End of Treatment (EOT) visit (Day 113) and may continue to the follow-up period for a final visit at Day 197, unless the subject withdraws consent, is lost to follow-up, or is enrolled in another clinical study.

The decision to discontinue dosing a subject due to an AE will be made on the basis of clinical severity and relatedness to the study drug. It is recommended that the Investigator consult with the Sponsor's medical monitor before removing the subject from the study.

Lost to Follow-Up

A subject will be considered lost-to-follow-up only if no contact has been established by the time the study is completed such that there is insufficient information to determine the subject's status on Day 197. Subjects refusing to return to the site or to continue participation in the study should be documented as “withdrawal of consent” rather than “lost to follow-up.” Investigators should document attempts to re-establish contact with missing subjects throughout the study period. If contact with a missing subject is re-established, the subject should not be considered lost-to-follow-up and any evaluations should resume according to the protocol (i.e., the subject should continue to the next scheduled visit relative to their Day 1 visit).

Permanent Discontinuation of Study Drug

A subject who is permanently discontinued from further receipt of study drug, regardless of the reason (withdrawal of consent, due to an AE, other reason), will be identified as having permanently discontinued treatment.

A subject who is permanently discontinued from receiving study drug will be asked to complete all procedures scheduled for the EOT visit (Day 113). A subject who is withdrawn from study drug treatment may continue to the follow-up period, unless the subject withdraws consent, is lost to follow-up, or is enrolled in another clinical study. Subjects who permanently discontinue treatment may either be considered to have completed the study or not to have completed the study.

Replacement of Subjects

Not applicable.

Treatment of Subjects

Description of Study Drug

Information on the study drugs are provided in Table 84.

TABLE 84
Study Drugs

| | Investigational Product | Placebo |
|---|---|---|
| Product Name | GBR830 | Placebo for GBR830 |
| Dosage Form | Investigational Product is supplied as | Placebo lyophilized formulation in |
| | lyophilized formulation in 10 mL vials | 10 mL vials that deliver 1 mL of SC |
| | that deliver 1 mL of SC solution at 150 | solution after reconstitution |
| | mg/mL after reconstitution | |
| Dosage | Loading dose: see Table 3 Maintenance | Loading dose: see Table 3 |
| | Dose: see Table 3 | Maintenance Dose: see Table 3 |
| Route of Administration | SC injection | SC injection |

Study Drug Materials and Management

Study Drug

A description of treatment groups and their respective dose regimens is provided in Table 85. All subjects will receive a loading dose consisting of 2 SC injections, and maintenance dosing consisting of 1 SC injection per dose, to maintain the blind.

TABLE 85
Treatment Groups and Dose Regimens Treatment Group

| | | | Maintenance Dose |
|---|---|---|---|
| | | | Schedule (Starting on |
| | Loading Dose (Day 1) | Maintenance Dose | Week 2) |
| Group 1 | 2 GBR 830 SC injections | 1 GBR 830 SC injection | GBR 830: Weeks 2, 4, |
| | (2 × 2 mL of 150 mg/mL | (1 × 2 mL of 150 mg/mL | 6, 8, 10, 12, and 14 |
| | formulation) | formulation) | |
| Group 2 | 2 GBR 830 SC injections | 1 GBR 830 SC injection | GBR 830: Weeks 4, 8 |
| | (2 × 2 mL of 150 mg/mL | (1 × 2 mL of 150 mg/mL | and 12 |
| | formulation) | formulation) | |
| 1 placebo SC injection (1 × 2 mL) | | Placebo: Weeks 2, 6, 10 and 14 | |
| Group 3 | 2 GBR 830 SC injections | 1 GBR 830 SC injection | GBR 830: Weeks 4, 8 |
| | (2 × 2 mL of 37.5 mg/mL | (1 × 2 mL of 37.5 | and 12 |
| | formulation) | mg/mL formulation) | |
| 1 placebo SC injection (1 × 2 mL) | | Placebo: Weeks 2, 6, 10 and 14 | |
| Group 4 | 2 placebo SC injections | 1 placebo SC injection | Placebo: Week 2, 4, 6, |
| | (2 × 2 mL) | (1 × 2 mL) | 8, 10, 12, and 14 |

Investigational Product

GBR830 is provided as lyophilized powder in a 10 mL glass vial. Each vial contains 192 mg of GBR830, 160 mg of sucrose, 3.1 mg of histidine, and 0.4 mg of polysorbate 80, and is designed to deliver 150 mg of GBR830 in 1.0 mL injection after reconstitution with 1.1 mL of sterile water for injection.

Placebo

Placebo is supplied as 200 mg of sucrose, 4 mg of histidine, and 0.5 mg of polysorbate 80. Each vial of GBR830 placebo is designed to be reconstituted with 1.3 mL of sterile water for injection to yield corresponding placebo.

Study Drug Storage

Investigational product is stored at +5° C.±3° C. (+35.6° F. to +46.4° F.), protected from light and moisture. Do not freeze or shake.

Study Drug Preparation

Appropriate aseptic technique should be used while preparing and administering the injection. The designated, trained, unblinded pharmacist (or designee) will prepare and dispense study drug for each subject according to the subject's IWRS/IVRS assignment.

Administration

The day of administration of the first dose of study drug (GBR 830 or placebo) is considered Day 1. Appropriate aseptic technique should be used while preparing and administering injections. Designated, trained, unblinded personnel (pharmacist or designee) will administer prepared study drug to maintain the blind. The monitoring done following dosing will be conducted by blinded study personnel.

The investigational product will be administered by a slow SC injection over 10 seconds. Subcutaneous injection sites should be alternated among the different quadrants of the abdomen (avoiding navel and waist areas), upper thighs, and upper arms, so that the same site is not injected for 2 consecutive dosing visits. To allow for adequate assessment of possible ISRs, study drug should be administered only into areas of normal-looking skin.

As with any antibody/medication, allergic reactions to dose administration are possible. Therefore, appropriate drugs and medical equipment to treat acute anaphylactic reactions must be immediately available, and study personnel must be trained to recognize and treat anaphylaxis.

Pharmacokinetic, Immunogenicity and Pharmacodynamic (Biomarker), and Assessments

Pharmacokinetic, Immunogenicity, and Pharmacodynamic (Biomarker) Blood Sampling Time Points and Allowed Windows

An overview of blood sampling time points for the rich PK group, the sparse PK group, biomarkers and immunogenicity assessments is provided in Table 86.

Blood samples will be collected as per routine phlebotomy procedures and at the time points specified below. Blood samples will be collected during the course of the study through an indwelling cannula placed in forearm veins or alternatively, by a fresh clean venipuncture using a disposable sterilized syringe and a needle. The cannulae will be maintained patent as per local practice; heparin should not be used. The actual sampling time will be recorded in the source documents and electronic case report form (eCRF). Actual collection times will be used during PK, PD (biomarker) and immunogenicity calculations.

TABLE 86
Pharmacodynamic (Biomarker), Pharmacokinetic,
and Immunogenicity Blood Sampling Time Points.

| | | | | | Allowed | |
|---|---|---|---|---|---|---|
| | Scheduled PD | Scheduled | Scheduled | Scheduled | Sampling | |
| | (Biomarker) | Rich PK | Sparse PK | Immunogenicity | Time Point | |
| Time Point | Blood Sampling | Group Blood | Group Blood | Blood Sampling | Window | |
| Screening | X | N/A | N/A | N/A | Screening | |
| Day 1 | X | X | N/A | X | Within 15 | |
| Predose | | | | | min prior to | |
| Day 1 (4 h post | N/A | X | N/A | N/A | ±10 | min |
| Dose 1) | | | | | | |
| Day 2 (24 h) | N/A | X | N/A | N/A | ±1 | h |
| Day 5 (96 h) | N/A | X | N/A | N/A | ±4 | h |
| Day 6 (120 h) | N/A | X | N/A | N/A | ±6 | h |
| Day 8 (168 h) | X | X | N/A | N/A | ±8 | h |
| Day 15 ± 1 day | X | X | X | X | Within 15 | |
| (336 h); | | | | | min prior to | |
| (Predose [symbol] | | | | | dosing | |
| Day 29 ± 1 day | X | X | X | N/A | Within 15 | |
| (672 h); | | | | | min prior to | |
| (Predose [symbol] | | | | | dosing | |
| Day 43 ± 1 day | N/A | X | X | N/A | Within 15 | |
| (1008 h); | | | | | min prior to | |
| (Predose Dose [symbol] | | | | | dosing | |
| Day 57 ± 1 day | X | X | X | X | Within 15 | |
| (1344 h, | | | | | min prior to | |
| (Predose [symbol] | | | | | dosing | |
| Day 71 ± 1 day | N/A | X | X | N/A | Within 15 | |
| (1680 h, | | | | | min prior to | |
| (Predose [symbol] | | | | | dosing | |
| Day 85 (2016 h, | X | X | X | X | Within 15 | |
| (Predose Dose [symbol] | | | | | min prior to | |
| | | | | | [symbol] | |
| Day 85 (2020 h) | N/A | X | N/A | N/A | ±10 | min |
| Day 86 (2040 h) | N/A | X | N/A | N/A | ±1 | h |
| Day 89 (2112 h) | N/A | X | N/A | N/A | ±4 | h |
| Day 90 (2136 h) | N/A | X | N/A | N/A | ±6 | h |
| Day 92 (2184 h) | N/A | X | N/A | N/A | ±8 | h |
| Day 99 ± 1 day | N/A | X | X | X | Within 15 | |
| (2352 h, | | | | | min prior to | |
| (Predose [symbol] | | | | | dosing | |
| Day 113 ± 1 day | X | X | X | X | ±24 | h |
| (2688 h) | | | | | | |
| Day 197 ± 5 days | N/A | X | X | X | ±5 | days |
| (4704 h) | | | | | | |

h = hours; min = minutes; N/A = not applicable; PD = pharmacodynamics; PK = pharmacokinetic.
Note:
All hours shown in column 1 of the table are in relation to the initial dose on Day 1. Subsequent doses are shown in parentheses in the first column of the table (e.g.predose Dose 2). The estimated amount of blood to be collected for each sample is: 3.5 mL for PK samples, 11.5 mL total for biomarkers, cytokines and IgE, and 5.0 mL for immunogenicity samples.
¹ Blood samples for biomarker assessments will be taken from a subset of subjects. Biomarkers to be analyzed include leukocyte subpopulation cell counts by flow cytometry; cytokines; total IgE; serum soluble OX40 and OX40 ligand. [symbol]
indicates data missing or illegible when filed

Pharmacokinetic Assessments

Blood samples (3.5 mL each) will be collected at appropriate time points defined in Table 86. An experimental population PK design will be used for the PK blood sampling. The subjects in the rich PK group and will have additional blood sampling between Days 1 to 8 (Week 1), and Days 85 to 92 (Week 12). Approximately 80 rich PK subjects will be randomized (in a 1:1:1:1 ratio) to the treatment arms. The subjects in the sparse PK group will have widespread sampling with fewer time points for each subject. A final sample will be collected on Day 197 for subjects that enter the follow-up period. Details of sample collection, processing and storage will be outlined in a separate laboratory manual. The samples will be shipped to the bioanalytical laboratory, as specified in the laboratory manual. Serum concentrations of GBR 830 will be quantified using a validated enzyme-linked immunosorbent assay (ELISA) method. In particular, in this method, microplate wells are pre-coated with the capture protein (OX40-His) as specific antigen for GBR 830. After blocking unspecific binding sites the plate was incubated with study samples, standards and quality control samples. GBR 830 binds to the immobilized antigen on the plate. After washing to remove the unbound antibody, detecting antibody (goat anti-human immunoglobulin G (hIgG) Fcγ Fragment specific conjugated with horseradish peroxidase (HRP)) is added and developed by a standard colorimetric tetramethylbenzidine (TMB) substrate. The absorbance at 450 nm is measured and a standard curve is generated by plotting absorbance versus the concentration of the GBR 830 standards using a four parameter regression model with 1/γ2 weighting. The concentration of GBR 830 in quality control samples or study samples was interpolated from the standard curve. In this case the calibration range is from 25000 ng/mg to 390 ng/mg.

Only the serum samples from subjects, belonging to treatment arms that received GBR 830 will be analyzed. In order to enable this, a designated person at the bioanalytical site will be unblinded.

Immunogenicity Assessments

Blood samples (5 mL each) will be collected at appropriate time points defined in Table 86 to detect the presence of anti-drug antibodies (ADA) to GBR 830, as per procedures similar to collection of PK samples. Antibodies generated against GBR 830 will be detected and confirmed using a validated ELISA method. Details of sample collection, processing and storage and shipment to the bioanalytical laboratory will be outlined in a separate laboratory manual.

Pharmacodynamic (Biomarker) Assessments (Optional)

Optional blood and skin samples from a subset of subjects (up to 10 sites with at least 2 subjects each) will be obtained at appropriate time points for biomarker assessments as defined in Table 86. A separate informed consent form will be provided at the initial Screening Visit for PD biomarker blood and skin biopsy sample collection.

Leukocyte Sub-population Cell Counts by Flow Cytometry

Blood samples (8.5 mL each) will be collected at appropriate time points defined in Table 86. The details of sample collection, processing and storage will be outlined in the laboratory manual.

Cytokines

Blood samples will be collected at appropriate time points defined in Table 86. The details of sample collection, processing and storage will be outlined in the laboratory manual.

Serum Biomarkers in Peripheral Blood

Blood samples will be collected at appropriate time points defined in Table 86. The details of sample collection, processing and storage will be outlined in the laboratory manual.

Total Immunoglobulin E

Subjects with AD often have elevated immunoglobulin E (IgE). Total IgE levels have been found to modestly correlate with AD severity and may be involved in the pathogenesis of the disease. Changes in total IgE reflects not only on AD, but atopy in general. Baseline IgE levels will be assessed for potential predictive value for treatment response. Post-treatment samples will be evaluated for effects of GBR 830 on total IgE. Blood samples will be collected at appropriate time points defined in Table 86. Detailed instructions for blood sample collection will be outlined in the laboratory manual.

Serum Soluble OX40 Ligand and Serum Soluble OX40

Blood samples (3.5 mL) for the estimation of soluble OX40L and soluble OX40 in serum will be collected at the time points specified in Table 86. The samples will be shipped to the bioanalytical laboratory, as specified in the laboratory manual. Serum concentrations of soluble OX40 L and soluble OX40 will be quantified using a suitable analytical method.

Assessment of Safety

Safety Parameters

Safety and tolerability of GBR 830 will be assessed, including AEs; SAES; TEAEs; anaphylactic events; ISRs; vital signs; physical examinations; electrocardiograms (ECGs).

Statistics

The Statistical Analysis Plan (SAP) will be written to provide details of the analysis, along with specifications for tables, listings, and figures to be produced. The SAP will be finalized before the database lock at the latest. If there are differences, the information in the SAP will supersede the information in the protocol. Any changes from the analyses planned in the SAP will be justified in the CSR.

All analyses will be performed by the Sponsor (or designee Contract Research Organization [CRO]) using SAS® software program version 9.3 or above. In general, all data will be summarized with descriptive statistics (number of subjects, mean, and standard deviation [SD], minimum, median, and maximum) for continuous variables and frequency and percentage for categorical variables.

Sample Size

A sample size of 78 completed subjects per group will provide 90% power to detect a difference of 23% between GBR 830 and placebo treatment in the percentage of subjects who achieved an IGA score of 0 or 1 at week 16, assuming that the percentages are 35% and 12% for GBR 830 and placebo, respectively. A 2-sided test at the 0.05 significance level will be considered.

Assuming a dropout rate of 20%, 98 subjects per arm will be randomized for a total of 392 subjects in 1:1:1:1 ratio.

Analysis Sets

Full Analysis Set

The Full Analysis Set (FAS) consists of all subjects who are randomized and received at least 1 dose of study medication. Based on the intent-to-treat principle, subjects will be analyzed according to the treatment group assigned.

Per Protocol Set

The Per Protocol Set (PPS) consists of all FAS subjects who complete the treatment period of 16 weeks and primary assessments as planned, and who have no major protocol deviations of eligibility or on-treatment study conduct.

Safety Analysis Set

The Safety Analysis Set (SAS) consists of all subjects who were randomized and took at least 1 dose of study medication. Subjects will be analyzed according to the treatment they received.

Pharmacokinetic Analysis Set

The Pharmacokinetic Analysis Set (PKAS) consists of the subset of the SAS population who received GBR 830 and for whom sufficient serum concentration data are available to facilitate derivation of PK parameters, do not have any major protocol deviation, and for whom the time of dosing and the time of sampling are known. Additional subjects may be excluded from the PKAS at the discretion of the pharmacokineticist. Any formal definitions for exclusion of subjects or time points from the PKAS will be documented in the SAP.

Endpoints

Primary Endpoint
Proportion of subjects with both IGA 0 to 1 (on a 5-point scale) and an IGA reduction from baseline of 2 points at Week 16.

Secondary Endpoint(s)

Efficacy Endpoints:
Proportion of subjects with EASI-75 (≥75% improvement from baseline) at Week 16 (key secondary endpoint).
Dose response of GBR 830 evaluated by percent change from baseline in EASI.
Percentage change in pruritus NRS scored on a scale of 0-10 from baseline, and number and percent of subjects with improvement of NRS from baseline at each time point investigated through Week 16.
Proportion of subjects who achieve an EASI 50 (50% improvement from baseline) response from baseline through Week 16.
Change in SCORAD from baseline.
Change in the DLQI from baseline through Week 16.
Change in GISS (erythema, infiltration/population, excoriations, lichenification) from baseline through Week 16.
Change in HADS from baseline through Week 16.
Change in POEM from baseline through Week 16.
Absolute and percent change in Patient Global Assessment of Disease from baseline through Week 16.
Absolute and percent change in Patient Global Assessment of Treatment from baseline through Week 16.
Assessment of sick leave and/or missed school days through Week 16. Safety Endpoints:
Incidence of TEAEs from baseline through Week 16.
Incidence of skin infection TEAEs requiring systemic treatment from baseline through Week 16.
Incidence of conjunctivitis TEAEs requiring systemic treatment from baseline through Week 16.
Incidence of treatment-emergent SAEs from baseline through Week 16.
Incidence of TEAEs leading to treatment discontinuation from baseline through Week 16.
Overall number of TEAEs and SAEs through Week 16.
Vital signs, clinical laboratory values, and ECG results monitored from baseline through Week 16.
Formation of ADA to GBR 830 to evaluate immunogenicity.

Pharmacokinetics Endpoints:

Cₘₐₓ, tₘₐₓ, AUC₀₋ₜₐᵤ, AUC from time 0 to the last measurable concentration (AUC₀₋ₜ), and other related parameters will be estimated using data from the rich PK group. C_{trough} values will be estimated using data from both rich and sparse PK groups.

Exploratory Endpoints

Data for exploratory endpoints will be captured at selected study sites. It is estimated that up to 10 sites with at least 2 subjects each will participate in the biomarker endpoints.

Exploratory endpoints are:
Messenger RNA (mRNA) expression of immune and barrier measures in involved and uninvolved skin biopsies at baseline, end of Week 8, and end of Week 16 in selected sites.
Biomarker analysis in GBR 830 responder and non-responder populations.
Change in the EQ-5D.
Change in Juniper ACQ-5 from baseline through Week 16.
Change in the SNOT-22 from baseline through Week 16.

Subject Disposition

Data on subject disposition (number of subjects enrolled, number of drop-outs, and reasons for drop-out), demographics (gender, age, height), and other baseline characteristics will be summarized. The safety, tolerability, PK, and other data from the study will be listed and summarized descriptively by treatment. The number (percentage) of subjects who were screened for the study (enrolled subjects, i.e., those who signed informed consent) and reasons for screen failure will be described.

Demographic and Other Baseline Characteristics

Demographics and other baseline characteristics will be summarized by treatment group for the FAS. Descriptive statistics will include number of subjects, mean, SD, minimum, median and maximum for continuous variables, and frequency and percentage for categorical variables.

Continuous demographic and baseline variables include age, height and body weight, and BMI; categorical variables include gender, race, and ethnicity.

Efficacy Analyses

The efficacy analyses will be conducted for all the subjects in the FAS using the treatment arm as assigned.

For the primary efficacy endpoint IGA and key secondary endpoint EASI 75, the Cochran-Mantel-Haenszel test adjusted by randomization strata (region, disease severity) will be used for the percentage of subjects with IGA of 0 or 1 at Week 16 or the percentage of subjects with EASI 75 at Week 16. To conserve the overall type I error at the 0.05 level, a fixed sequence testing approach will be implemented. Testing will be done in the order described below:
GBR 300 mg q2w vs placebo comparison for IGA will first be tested at the 0.05 significance level. If it is significant, continue; otherwise stop.
GBR 300 mg q4w vs placebo comparison for IGA will be tested at the 0.05 significance level. If it is significant, continue; otherwise stop.
GBR 300 mg q2w vs placebo comparison for EASI 75 will be tested at the 0.05 significance level. If it is significant, continue; otherwise stop.
GBR 300 mg q4w vs placebo comparison for EASI 75 will be tested at the 0.05 significance level.

Details of efficacy, safety and PK analyses will be specified in the SAP and included in the CSR.

Biomarker analyses will be described in a Biomarker Analysis Plan. Biomarker data will be published in a separate report.

Detailed statistical methods, including methods for the handling of missing data for analyzing the secondary and exploratory endpoints and the exploratory analyses on biomarkers will be fully described in the SAP and Biomarker Analyses Plan.

Pharmacokinetic, Pharmacodynamic (Biomarker), and Immunogenicity Analyses

Pharmacokinetic Analyses

The PKAS will be used for the PK analyses. The PK parameters will be derived for individual subject by non-compartmental analysis using appropriate validated software. Pharmacokinetic parameters (Cₘₐₓ, tₘₐₓ, AUC₀₋ₜₐᵤ, AUC₀₋ₜ, and other related parameters) will be estimated based on data from the rich PK group.

Estimates of C_{trough} will be derived using the data from both rich PK group and sparse PK group.

The PK parameters will be summarized in tabular and graphic form. Details of the PK analysis will be specified in the SAP.

The serum concentration data from both rich and sparse PK group may be used for the population PK analysis and will be reported separately.

Immunogenicity Analyses

The number and percent incidence of positive and negative ADA status of subjects by treatment, and time points will be provided. Titers and neutralizing potential of confirmed positive samples will be reported. Details will be provided in the SAP.

Pharmacodynamic (Biomarker) Analyses Summary statistics will be provided for PD biomarkers.

A Biomarker Analysis Plan will be generated and data will be published separately from the CSR.

Safety Analyses

All safety analyses will be performed on the SAS population, according to the actual treatment received. Adverse events will be summarized by system organ class and preferred term.

Subjects will be counted only once for each preferred term, system organ class, and by the highest severity of an event. Laboratory evaluations will be summarized with descriptive statistics at each visit, and change from baseline summarized for each post-baseline visit. Laboratory measurements will also be summarized based on the number and percentage of subjects above or below a pre-specified threshold for each test. Details will be presented in the SAP.

Extent of Exposure

The duration of exposure to study drug will be summarized separately by respective treatment group for SAS subjects.

Adverse Events

Adverse events (AEs) will be coded using the Medical Dictionary for Regulatory Activities (MedDRA). The number and percentage of AEs, SAES, AEs leading to discontinuation, and AEs related to investigational product will be summarized by system organ class, preferred term and treatment group. Subjects will be counted only once for each preferred term, system organ class, and by the highest severity of an event. The number and percentage of AEs by severity will also be summarized. All AEs will be displayed in listings.

Laboratory Values

For quantitative laboratory measurements descriptive statistics will be used to summarize results and change from baseline by treatment group and time point. Shifts in laboratory tests relative to reference ranges from baseline to the worst post-baseline value during treatment will also be tabulated. All laboratory data will be displayed in listings.

Vital Signs

Descriptive statistics will be used to summarize vital sign results and changes from baseline by treatment group and time. Values of potential clinical significance will be tabulated. All vital signs data will be displayed in listings.

Shift tables will present changes from baseline (categorized as normal; abnormal, not clinically significant; and abnormal, clinically significant) to end of treatment.

Electrocardiograms

All ECG variables will be presented by visit. Descriptive statistics for ECG parameters and changes from baseline will be presented by treatment group.

Shift tables will present changes from baseline in ECG interpretation (categorized as normal; abnormal, not clinically significant; and abnormal, clinically significant) to end of treatment.

Physical Examination

Descriptive statistics will be used to summarize findings of potential clinical significance and will be listed.

1. An anti-OX40 antagonist antibody for use in the treatment of an OX40-mediated disorder, wherein said antibody is suitable for intravenous administration at least a single dose of at least 300 mg; or
wherein said antibody is suitable for subcutaneous administration at least a single dose of 50 mg.

2. The antibody of claim 1, wherein said antibody is suitable for intravenous administration
(a) at a single dose comprised between about 10 mg/kg of a subject body weight and about 50 mg/kg of a subject body weight; or
(b) at a single dose equal to or less than 3 g; or
(c) at multiple doses of about 1 mg/kg of a subject body weight to about 30 mg/kg of a subject body weight administrated for at least two consecutive weeks at least once a week.

3. The antibody of claim 1, wherein said antibody is suitable for subcutaneous administration
(a) at a single dose comprised between about 50 mg and about 1 g; or
(b) at a loading dose comprised between about 50 mg and about 1.5 g on Day 1, followed by at least one maintenance dose comprised between about 20 mg and about 1 g, starting on a day comprised between Day 10 and Day 40.

4. The antibody of claims 1 and 2, wherein said antibody is suitable for intravenous administration of a single dose of:
(a) about 20 mg/kg of a subject body weight and wherein following administration, pharmacokinetics parameters of said antibody comprise Cmax comprised between about 400 mcg/mL and about 800 mcg/mL and t1/2 comprised between about 200 hours and about 500 hours; or
(b) about 40 mg/kg of a subject body weight and wherein following administration, pharmacokinetics parameters of said antibody comprise Cmax comprised between about 900 mcg/mL and about 1300 mcg/mL and t1/2 comprised between about 300 hours and about 600 hours; or
(c) about 600 mg, and wherein following administration, pharmacokinetics parameters of said antibody comprise Cmax comprised between about 100 μg/mL and about 300 μg/mL and t1/2 comprised between about 200 hours and about 500 hours.

5. The antibody of claims 1 and 2, wherein said antibody is suitable for intravenous administration at a multiple dose of:
(a) about 10 mg/kg of a subject body weight for at least 6 consecutive weeks at least once a week, and wherein following administration, pharmacokinetics parameters of said antibody comprise Cmax comprised between about 200 mcg/mL and about 400 mcg/mL at week 1, comprised between about 400 mcg/mL and about 700 mcg/mL at week 4, and comprised between about 500 mcg/mL and about 800 mcg/mL at week 6 and t1/2 comprised between about 200 hours and about 500 hours at week 6; or
(b) about 20 mg/kg of a subject body weight, for at least 6 consecutive weeks at least once a week, and wherein following administration, pharmacokinetics parameters of said antibody comprise Cmax comprised between about 400 mcg/mL and about 700 mcg/mL at week 1, comprised between about 900 mcg/mL and about 1300 mcg/mL at week 4, and comprised between about 1000 mcg/mL and about 1400 mcg/mL at week 6 and t1/2 comprised between about 300 hours and about 600 hours at week 6.

6. The antibody of claims 1 and 3, wherein when said antibody is suitable for subcutaneous administration at a single dose of:
(a) about 600 mg, and wherein following administration, pharmacokinetics parameters of said antibody comprise Cmax comprised between about 30 μg/mL and about 90 μg/mL and t1/2 comprised between about 200 hours and about 500 hours; or
(b) about 75 mg, and wherein following administration, pharmacokinetics parameters of said antibody comprise Cmax comprised between about 2 μg/mL and about 18 μg/mL and t1/2 comprised between about 100 hours and about 400 hours.

7. The antibody of claims 1 and 3, wherein said antibody is suitable for subcutaneous administration
(a) at a loading dose comprised between about 300 mg and about 1 g on Day 1, followed by at least one maintenance dose comprised between about 100 mg and about 600 mg, starting on a day comprised between Day 10 and Day 20, or
(b) at a loading dose comprised between about 300 mg and about 1 g on Day 1, followed by at least one maintenance dose comprised between about 100 mg and about 600 mg, starting on a day comprised between Day 20 and Day 40, or
(c) at a loading dose comprised between about 50 mg and about 300 mg on Day 1, followed by at least one maintenance dose comprised between about 20 mg and about 150 mg, starting on a day comprised between Day 20 and Day 40.

8. The antibody of claim 7, wherein said maintenance dose is administrated every n days thereafter, wherein n is comprised between 10 days and 20 days; or comprised between 20 days and 40 days.

9. The antibody of anyone of claims 7 and 8, wherein said antibody is suitable for subcutaneous administration
(a) at loading dose of about 600 mg on Day 1, followed by a maintenance dose of about 300 mg starting at Day 15 every 2 weeks; or
(b) at loading dose of about 600 mg on Day 1, followed by a maintenance dose of about 300 mg starting at Day 29 every 4 weeks; or
(c) at loading dose of about 150 mg on Day 1, followed by a maintenance dose of about 75 mg starting at Day 29 every 4 weeks.

10. The antibody of anyone of claims 7 to 9, wherein said antibody is suitable for subcutaneous administration to a subject wherein the subject has at least one characteristic selected from the group comprising:
(a) having AD involvement of ≥10% body surface area (BSA) at screening and baseline.
(b) having EASI score equal to or greater than about 12 at screening or equal to or greater than about 16 at baseline;
(c) having IGA score equal to or greater than about 3 at screening and at baseline;
(d) having Baseline Pruritus Numerical Rating Scale (NRS) score for maximum itch intensity equal to or greater than about 3 over the previous 24 hours;

11. The antibody of any one of the preceding claims, wherein said OX40-mediate disorder is selected from the group comprising infections (viral, bacterial, fungal and parasitic, endotoxic shock associated with infection, arthritis, rheumatoid arthritis, asthma, bronchitis, influenza, respiratory syncytial virus, pneumonia, COPD, idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coehac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, wherein atopic dermatitis is mild, or mild-to-moderate, or moderate, or moderate-to-severe, or severe, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

12. The antibody of any one of claims 1 to 11 wherein said OX40-mediated disorder selected from the group comprising atopic dermatitis wherein atopic dermatitis is mild, or mild-to-moderate, or moderate, or moderate-to-severe, or severe, rheumatoid arthritis, autoimmune uveitis, multiple sclerosis, lupus (such as systemic lupus erythematosus), ulcerative colitis, scleroderma and graft-versus-host disease (GVHD) and hidradenitis.

13. The antibody of claims 7 to 10, wherein the OX40-mediate disorder moderate-to-severe atopic dermatitis.

14. The antibody of any one of the preceding claims wherein the antibody is identified by the CAS Registry Number: 2126777-87-3.

15. A stable pharmaceutical formulation comprising the antibody of any one of claims 1 to 14.
